(19) United States
(12) Patent Application Publication
(10) Pub. No.: US 2012/0009177 A1
(43) Pub. Date: Jan. 12, 2012

(54) GENE EXPRESSION MARKERS FOR PREDICTING RESPONSE TO INTERLEUKIN-6 RECEPTOR-INHIBITING MONOCLONAL ANTIBODY DRUG TREATMENT

(75) Inventors: Adam Platt, Congleton (GB); Jianmei Wang, Welwyn Garden City (GB); Guiyuan Lei, Welwyn Garden City (GB); Laurent Essioux, Attenschwiller (FR); Wei-min Liu, Dublin, CA (US); P. Mickey Williams, Great Falls, VA (US)

(73) Assignee: Roche Molecular Systems, Inc., Pleasanton, CA (US)

(21) Appl. No.: 13/154,158

(22) Filed: Jun. 6, 2011

Related U.S. Application Data
(60) Provisional application No. 61/352,285, filed on Jun. 7, 2010.

(51) Int. Cl. A61K 39/395; A61P 37/02; C40B 40/06; C12Q 1/68; C40B 30/04

(52) U.S. Cl. 424/130.1; 435/6.12; 435/6.11; 506/9; 506/16

(57) ABSTRACT

This invention provides methods, compositions, and kits relating to gene product biomarkers where gene expression levels are correlated with therapeutic response of rheumatoid arthritis patients to treatment with an IL-6 receptor antagonist, such as an IL6-R antibody. The methods, compositions, and kits of the invention can be used to identify rheumatoid arthritis patients who are likely, or not likely, to respond to IL-6 receptor antagonist treatments.

### CROSS-REFERENCE TO RELATED APPLICATIONS

This application claims benefit of U.S. provisional application No. 61/352,285, filed Jun. 7, 2010, which is herein incorporated by reference for all purposes.

### BACKGROUND OF THE INVENTION

Tocilizumab is the first humanized interleukin-6 receptor (IL-6R)-inhibiting monoclonal antibody that has been developed to treat rheumatoid arthritis. As with other treatments, the antibody exhibits a range of therapeutic efficacy in patients. Thus, there is a need to determine those patients that are more likely to respond positively to treatment with tocilizumab and/or patients that are likely to not respond to treatment. The present invention addresses this need.

### BRIEF SUMMARY OF THE INVENTION

The invention is based, in part, on the discovery of changes in gene expression that are associated with a positive therapeutic response to treatment with an agent that modulate IL-6-mediated signal transduction, such as an anti-IL-6 antibody that inhibits transduction or an IL-6R-inhibiting monoclonal antibody such as tocilizumab.

Thus, in one aspect, the invention provides a method of identifying a rheumatoid arthritis patient that is likely to respond to treatment with tocilizumab; or of identifying a patient that is likely not to respond to treatment with tocilizumab; wherein the method comprises identifying the levels of expression of a gene set forth in Table 1, Table 2, or Table 3. Such genes can be identified using a variety of techniques, including array probe sets and amplification techniques. The level of expression of the marker gene is then compared to the expression level shown in the data set used to establish a correlation.

In a further aspect, the invention provides, a kit for predicting the therapeutic response of a rheumatoid arthritis patient to a treatment regimen that comprises administration of an IL-6R antibody such as tocilizumab. In some embodiments, the kit also includes an electronic device or computer software to compare the marker gene expression level of a biomarker gene set forth in Table 1, Table 2, or Table 3 from the patient to a dataset. The endpoint for evaluating therapeutic response can be any symptom of rheumatoid arthritis, e.g., the endpoints evaluated in Example 1.

In some embodiments, the marker gene is any one of the genes set forth in Table 1. In some embodiments, the marker genes are at least two genes set forth in Table 1. Thus, in some embodiments any one of from 2 to 20, 30, 40, 50, 60, 70, 80, or all of the genes set forth in Table 1.

In some embodiments, the marker gene is any one of the genes set forth in Table 2. In some embodiments, the marker genes are at least two genes set forth in Table 2. Thus, in some embodiments any one of from 2 to 20, 30, 40, 50, 60, 70, 80, or all of the genes set forth in Table 2.

In some embodiments, the marker gene is any one of the genes set forth in Table 3. In some embodiments, the marker genes are at least two genes set forth in Table 3. Thus, in some embodiments any one of from 2 to 20, 30, 40, 50, 60, 70, 80, or all of the genes set forth in Table 3.

In some embodiments, the step of determining the level of expression of the biomarker gene comprises measure the level of RNA expressed by the marker gene. The amount of RNA expressed may be determined, e.g., using an amplification area reaction such as qPCR, or by using a probe array. For example, a nucleic acid array forming a probe set may be used to detect RNA expressed of the biomarker gene. RNA expression levels are typically determined by measuring the level of cDNA transcribed from the RNA isolated from the patient. RNA expression levels can be determined using known probesets to quantify expression level. As known in the art, such probes sets may comprises multiple probes that hybridize to the target sequence of interest. Alternatively, expression of a marker gene can be determined by measuring the level of expression of a protein encoded by the gene.

The levels of expression are compared to standard control data, e.g., the expression data set generated in Example 1 and 2. An increased level of expression of the marker gene or decreased level of expression of the biomarker gene may be determined by using statistical models for determining whether expression of the biomarker gene is indicative of therapeutic response of a patient to treatment with an IL-6R antibody such as tocilizumab. In some the invention provides an electronic device or computer software that employs the use of a statistical model to determine likelihood of therapeutic responses.

In some embodiments, the levels of expression of genes set forth in Table 5 are evaluated to identify rheumatoid arthritis patients that are likely to be responsive, or unresponsive, to treatment with an IL-6R antagonist such as tocilizumab. In typical embodiments, anywhere from 2 to 10, 20, 30, 40, 50, 60, 70, 80, or 90, or all of the genes in column C, column D, column E, column F, column G, column H, column I, or column J are analyzed to determined likelihood of a therapeutic response.

### DETAILED DESCRIPTION OF THE INVENTION

As used herein, a “positive therapeutic response” or “therapeutic benefit” refers to an improvement in, and/or delay in the onset of, any symptom of rheumatoid arthritis.

As used herein “negative therapeutic response” refers to a lack of improvement of one or more symptoms of rheumatoid arthritis.

An “interleukin-6 receptor (IL-6R) inhibiting antibody” refers to an antibody to IL-6 receptor where the antibody binds to IL-6 receptor and antagonizes (i.e., inhibits) IL-6 receptor activity. An example of such an antibody is tocilizumab, a humanized IL-6R monoclonal antibody (see, e.g., Sato et al., Cancer Res 1993; 53: 851-6; and U.S. Pat. No. 7,479,543) that is used for the treatment of rheumatoid arthritis.

In the current invention, a “gene set forth in Table 1” refers to the gene that corresponds to the probesets annotated in Table 1. Similarly, a “gene set forth in” Tables 2, 3, or 5 refers to the gene that corresponds to the probesets annotated in the respective Table. For Tables 1-3, the “Representative Public ID” is listed as the accession number Table 1. The “Representative Public ID” is the accession number of a representative sequence. For consensus-based probe sets, the representative sequence is only one of several sequences (sequence sub-clusters) used to build the consensus sequence in the probe set used in the Examples and it is not directly used to derive the probe sequences. The representative sequence is chosen during array design as a sequence that is best associated with the transcribed region being interrogated by the probe set. As understood in the art, there are naturally occurring polymorphisms for many gene sequences. Genes that are naturally occurring allelic variations for the purposes of this invention are those genes encoded by the same genetic locus. The proteins encoded by allelic variations of a gene set forth in Table 1, Table 2, or Table 3 typically have at least 95% amino acid sequence identity to one another, i.e., an allelic variant of a gene indicated in Table 1, Table 2, or Table 3 typically encodes a protein product that has at least 95% identity, often at least 96%, at least 97%, at least 98%, or at least 99%, or greater, identity to the amino acid sequence encoded by the nucleotide sequence denoted by the accession number shown in the Table for that gene. For example, an allelic variant of a gene encoding Eph receptor B2 (gene: EPHB2, representative accession number AF025304) typically has at least 95% identity, often at least 96%, at least 97%, at least 98%, or at least 99%, or greater, to the Eph receptor b2 protein encoded by the sequence available under accession number AF025304.

The terms “identical” or “100% identity,” in the context of two or more nucleic acids or proteins refer to two or more sequences or subsequences that are the same sequences. Two sequences are “substantially identical” or a certain percent identity if two sequences have a specified percentage of amino acid residues or nucleotides that are the same (i.e., 70% identity, optionally 75%, 80%, 85%, 90%, or 95% identity, over a specified region, or, when not specified, over the entire sequence), when compared and aligned for maximum correspondence over a comparison window, or designated region as measured using known sequence comparison algorithms, e.g., BLAST using the default parameters, or by manual alignment and visual inspection.

A “gene product” or “gene expression product” in the context of this invention refers to an RNA or protein encoded by the gene.

The term “evaluating a biomarker” in a patient that has rheumatoid arthritis refers to determining the level of expression of a gene product encoded by a gene, or allelic variant of the gene, listed in Table 1, Table 2, Table 3, or Table 5. Typically, the RNA expression level is determined.

### INTRODUCTION

The invention is based, in part, on the identification of specific genes/transcripts whose gene expression level, prior to drug dosing or 8 weeks subsequent to dosing, are correlated with response to tocilizumab.

The invention therefore relates to measurement of expression level of a biomarker prior to the patient receiving the drug. In some embodiments, probes to detect such transcripts may be applied in the form of a diagnostic device to predict which rheumatoid arthritis patients will respond or not respond to an IL-6 receptor antagonist such as an IL-6 receptor antagonizing antibody, e.g., tocilizumab. Transcripts may also be measured to predict which RA patients will respond tocilizumab at a later time point. Further, the identification of proteins/metabolites and/or related transcripts and associated product that are linked by pathway or cell type or tissue expression to the transcripts identified herein in the Examples section can be used as alternative biomarkers for measurement of response to tocilizumab.

The expression levels of any gene expression product of one of the genes set forth in Table 1, Table 2, or Table 3 may be measured, however, typically expression of multiple genes is assessed. Gene expression levels may be measured using any number of methods known in the art. In typical embodiments, the method involves measuring the level of RNA. RNA expression can be quantified using any method, e.g., employing a quantitative amplification method such as qPCR. In other embodiments, the methods employ array-based assays. In still other embodiments, protein products may be detected. The gene expression patterns are determined using a whole blood or peripheral blood lymphocyte samples from the patient.

In some embodiments, gene products, typically RNA, encoded by a gene that is in the same pathway as a biomarker shown in Table 1, Table 2, or Table 3 may be quantified. In some embodiments, at least one of the biomarkers that is evaluated to identify a rheumatoid arthritis patient that is a candidate for treatment with tocilizumab is selected from the group consisting of JAM3, CD41, CD61, ephrin receptor B2. In some embodiments, at least one of the biomarkers selected for evaluation is JAM3, CD41, CD61, and a second biomarker evaluated is ephrin receptor B2. In some embodiments, a biomarker that is evaluated in a patient is a component of the inflammasome, caspase 1, caspase 5, IL-1 receptor, or CARD16. In some embodiments, at least one of the biomarkers that is evaluated is serine palmitoyltransferase long chain base subunit 2 or sphingosine-1-phosphate (S1P), ceramide or related sphingolipids.

In some embodiments, the methods of the invention comprise analyzing gene expression products of two or more biomarkers of Table 5 that have a value over “0” shown in one of columns C-J. Such biomarkers may be used in combination to predict likelihood of a rheumatoid arthritis patient's response to treatment in an IL-6R antagonist such as tocilizumab. Thus, for example, analysis of gene expression levels of at least two biomarkers, preferably three, four, five, or any number up to 100 of the biomarkers having a value above “0” in column C can be used in combination to predict response to treatment is tocilizumab. Similarly, at least two biomarkers, preferably three, four, five, or more, or all of the biomarkers from column D that have values above “0” can be analyzed for expression levels to identify rheumatoid arthritis patients likely to be responsive, or not responsive, to treatment with an IL-6R antagonist such as tocilizumab. In typical embodiments, those expression levels of those genes that have lower numbers, are evaluated. Thus, for example, a gene in column C that has a value of 1, 2, 3, 4, 5, 6, 7, 8, 9, 10, for example, is typically included in the analysis of gene expression. In some embodiments, the methods of the invention comprise analyzing expression level of two or more genes in column C; and analyzing expression levels of two or more genes in column D, or two or more genes in column E, etc.

In Table 5, the column “ID” refers to a probeset for the corresponding gene (Table 5B). One of skill understands that the probeset annotation in Table 5B and column L of Table 5A can be obtained through the database of the maker of the chip used for this analysis (Affymetrix).

### Methods for Quantifying RNA

The quantity of RNA encoded by a gene set forth in Table 1 can be readily determined according to any method known in the art for quantifying RNA. Various methods involving amplification reactions and/or reactions in which probes are linked to a solid support and used to quantify RNA may be used. Alternatively, the RNA may be linked to a solid support and quantified using a probe to the sequence of interest.

An “RNA nucleic acid sample” analyzed in the invention is obtained from peripheral blood lymphocytes. An “RNA nucleic acid sample” comprises RNA, but need not be purely RNA, e.g., DNA may also be present in the sample. Techniques for obtaining an RNA sample from peripheral blood lymphocytes are well known in the art.

In some embodiments, the target RNA is first reverse transcribed and the resulting cDNA is quantified. In some embodiments, RT-PCR or other quantitative amplification techniques are used to quantify the target RNA. Amplification of cDNA using PCR is well known (see U.S. Pat. Nos. 4,683,195 and 4,683,202; PCR PROTOCOLS: A GUIDE TO METHODS AND APPLICATIONS (Innis et al., eds, 1990)). Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol Biotechnol. 20(2):163-79 (2002). Alternative method for determining the level of a mRNA of interest in a sample may involve other nucleic acid amplification methods such as ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art.

In general, quantitative amplification is based on the monitoring of the signal (e.g., fluorescence of a probe) representing copies of the template in cycles of an amplification (e.g., PCR) reaction. One method for detection of amplification products is the 5′-3′ exonuclease “hydrolysis” PCR assay (also referred to as the TaqMan™ assay) (U.S. Pat. Nos. 5,210,015 and 5,487,972; Holland et al., PNAS USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the “TaqMan™” probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

Another method of detecting amplification products that relies on the use of energy transfer is the “beacon probe” method described by Tyagi and Kramer, Nature Biotech. 14:303-309 (1996), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched. When employed in PCR, the molecular beacon probe, which hybridizes to one of the strands of the PCR product, is in “open conformation,” and fluorescence is detected, while those that remain unhybridized will not fluoresce (Tyagi and Kramer, Nature Biotechnol. 14: 303-306 (1996)). As a result, the amount of fluorescence will increase as the amount of PCR product increases, and thus may be used as a measure of the progress of the PCR. Those of skill in the art will recognize that other methods of quantitative amplification are also available.

Various other techniques for performing quantitative amplification of nucleic acids are also known. For example, some methodologies employ one or more probe oligonucleotides that are structured such that a change in fluorescence is generated when the oligonucleotide(s) is hybridized to a target nucleic acid. For example, one such method involves is a dual fluorophore approach that exploits fluorescence resonance energy transfer (FRET), e.g., LightCycler™ hybridization probes, where two oligo probes anneal to the amplicon. The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: Scorpions™ probes (e.g., Whitcombe et al., Nature Biotechnology 17:804-807, 1999, and U.S. Pat. No. 6,326,145), Sunrise™ (or Amplifluor™) probes (e.g., Nazarenko et al., Nuc. Acids Res. 25:2516-2521, 1997, and U.S. Pat. No. 6,117,635), and probes that form a secondary structure that results in reduced signal without a quencher and that emits increased signal when hybridized to a target (e.g., Lux Probes™).

In other embodiments, intercalating agents that produce a signal when intercalated in double stranded DNA may be used. Exemplary agents include SYBR GREEN™ and SYBR GOLD™. Since these agents are not template-specific, it is assumed that the signal is generated based on template-specific amplification. This can be confirmed by monitoring signal as a function of temperature because melting point of template sequences will generally be much higher than, for example, primer-dimers, etc.

In other embodiments, the mRNA is immobilized on a solid surface and contacted with a probe, e.g., in a dot blot or Northern format. In an alternative embodiment, the probe(s) are immobilized on a solid surface and the mRNA is contacted with the probe(s), for example, in a gene chip array. A skilled artisan can readily adapt known mRNA detection methods for use in detecting the level of mRNA encoding the biomarkers or other proteins of interest.

In some embodiments, microarrays, e.g., are employed. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261. Although a planar array surface is often employed the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device.

Primer and probes for use in amplifying and detecting the target sequence of interest can be selected using well-known techniques.

In the context of this invention, “determining the levels of expression” of an RNA interest encompasses any method known in the art for quantifying an RNA of interest.

### Detection of Protein Levels

In some embodiments, e.g., where the expression level of a protein encoded by a biomarker gene set forth in Table 1 is measured. Often, such measurements may be performed using immunoassays. Although the protein expression level may be determined using a cellular sample, such as a peripheral blood lymphocyte sample, the protein expression is typically determined using a serum sample.

A general overview of the applicable technology can be found in Harlow & Lane, Antibodies: A Laboratory Manual (1988) and Harlow & Lane, Using Antibodies (1999). Methods of producing polyclonal and monoclonal antibodies that react specifically with an allelic variant are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975)). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

Polymorphic alleles can be detected by a variety of immunoassay methods. For a review of immunological and immunoassay procedures, see Basic and Clinical Immunology (Stites & Terr eds., 7th ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Terr, eds., 7th ed. 1991).

Commonly used assays include noncompetitive assays, e.g., sandwich assays, and competitive assays. Typically, an assay such as an ELISA assay can be used. The amount of the polypeptide variant can be determined by performing quantitative analyses.

Other detection techniques, e.g., MALDI, may be used to directly detect the presence of proteins correlated with treatment outcomes.

### Devices and Kits

In a further aspect, the invention provides diagnostic devices and kits for identifying gene expression products associated with improved responsiveness of a rheumatoid arthritis patient to a therapeutic agents that antagonizes IL-6 receptor signaling, such as an IL-6R antibody, e.g., tocilizumab.

In some embodiments, a diagnostic device comprises probes to detect at least 2, 3, 4, 5, 6, 7, 8, 9, 10, 15, 20, 50, 60, 70, or 80, or all of, the gene expression products set forth in Table 1. In some embodiments, the present invention provides oligonucleotide probes attached to a solid support, such as an array slide or chip, e.g., as described in DNA Microarrays: A Molecular Cloning Manual, 2003, Eds. Bowtell and Sambrook, Cold Spring Harbor Laboratory Press. Construction of such devices are well known in the art, for example as described in US Patents and Patent Publications U.S. Pat. No. 5,837,832; PCT application WO95/11995; U.S. Pat. No. 5,807,522; U.S. Pat. Nos. 7,157,229, 7,083,975, 6,444,175, 6,375,903, 6,315,958, 6,295,153, and 5,143,854, 2007/0037274, 2007/0140906, 2004/0126757, 2004/0110212, 2004/0110211, 2003/0143550, 2003/0003032, and 2002/0041420. Nucleic acid arrays are also reviewed in the following references: Biotechnol Annu Rev 8:85-101 (2002); Sosnowski et al, Psychiatr Genet 12(4):181-92 (December 2002); Heller, Annu Rev Biomed Eng 4: 129-53 (2002); Kolchinsky et al, Hum. Mutat 19(4):343-60 (April 2002); and McGail et al, Adv Biochem Eng Biotechnol 77:21-42 (2002).

An array can be composed of a large number of unique, single-stranded polynucleotides, usually either synthetic antisense polynucleotides or fragments of cDNAs, fixed to a solid support. Typical polynucleotides are preferably about 6-60 nucleotides in length, more preferably about 15-30 nucleotides in length, and most preferably about 18-25 nucleotides in length. For certain types of arrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length.

A person skilled in the art will recognize that, based on the known sequence information, detection reagents can be developed and used to assay any gene expression product set forth in Table 1, Table 2, or Table 3 and that such detection reagents can be incorporated into a kit. The term “kit” as used herein in the context of biomarker detection reagents, are intended to refer to such things as combinations of multiple biomarker detection reagents, or one or more biomarker detection reagents in combination with one or more other types of elements or components (e.g., other types of biochemical reagents, containers, packages such as packaging intended for commercial sale, substrates to which biomarker detection reagents are attached, electronic hardware components, etc.). Accordingly, the present invention further provides biomarker detection kits and systems, including but not limited to, packaged probe and primer sets (e.g., TaqMan probe/primer sets), arrays/microarrays of nucleic acid molecules where the arrays/microarrays comprise probes to detect the level of biomarker transcript, and beads that contain one or more probes, primers, or other detection reagents for detecting one or more biomarkers of the present invention. The kits can optionally include various electronic hardware components; for example, arrays (“DNA chips”) and microfluidic systems (“lab-on-a-chip” systems) provided by various manufacturers typically comprise hardware components. Other kits (e.g., probe/primer sets) may not include electronic hardware components, but may be comprised of, for example, one or more biomarker detection reagents (along with, optionally, other biochemical reagents) packaged in one or more containers.

In some embodiments, a biomarker detection kit typically contains one or more detection reagents and other components (e.g. a buffer, enzymes such as DNA polymerases) necessary to carry out an assay or reaction, such as amplification for detecting the level of biomarker transcript. A kit may further contain means for determining the amount of a target nucleic acid, and means for comparing the amount with a standard, and can comprise instructions for using the kit to detect the biomarker nucleic acid molecule of interest. In one embodiment of the present invention, kits are provided which contain the necessary reagents to carry out one or more assays to detect one or more biomarkers disclosed herein. In one embodiment of the present invention, biomarker detection kits/systems are in the form of nucleic acid arrays, or compartmentalized kits, including microfluidic/lab-on-a-chip systems.

Biomarker detection kits/systems may contain, for example, one or more probes, or pairs or sets of probes, that hybridize to a nucleic acid molecule encoded by a gene set forth in Table 1, Table 2, or Table 3. In some embodiments, the presence of more than one biomarker can be simultaneously evaluated in an assay. For example, in some embodiments probes or probe sets to different biomarkers are immobilized as arrays or on beads. For example, the same substrate can comprise biomarkers probes for detecting at least 1, 2, 3, 4, 5, 6, 7, 8, 9, 10, 15, or or more of the biomarkers set forth in Table 1, Table 2, or Table 3.

Using such arrays or other kits/systems, the present invention provides methods of identifying the biomarkers described herein in a test sample. Such methods typically involve incubating a test sample of nucleic acids obtained from peripheral blood lymphocytes from a patient with an array comprising one or more probes that selectively hybridizes to a nucleic acid encoded by a gene set forth in Table 1, Table 2, or Table 3. Conditions for incubating a biomarker detection reagent (or a kit/system that employs one or more such biomarker detection reagents) with a test sample vary. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect a biomarker set forth in Table 1, Table 2, or Table 3.

A biomarker detection kit of the present invention may include components that are used to prepare nucleic acids from a test sample for the subsequent amplification and/or detection of a biomarker nucleic acid molecule.

Correlating Gene Expression Levels with Therapeutic Response

The present invention provides methods of determining the levels of a gene expression product to evaluate the likelihood that a rheumatoid arthritis patient will respond to treatment with an IL-6R antibody, such as tocilizumab. Either female or male rheumatoid arthritis patients can be analyzed for gene expression levels.

The presence of certain markers, e.g., base line expression markers in Table 1 that are associated with an improvement in therapeutic outcomes, are indicative of patients who are expected to exhibit a positive therapeutic response to treatment with an IL-6R antibody, such as tocilizumab. Typically, the likelihood of the positive therapeutic response is increased with increasing amounts of the gene expression marker.

Similarly, a patient may have a gene expression marker, e.g., baseline expression of a biomarker set forth in Table 1, that is associated with a negative therapeutic outcome.

Accordingly, such a patient is not likely to response to IL-6R antibody, e.g., tocilizumab. Typically, the likelihood of the negative therapeutic response is increased with increased amount of the biomarker.

In Tables 1, 2, and 3, the “co-efficient” column represents the effect of the gene expression value on the response measured by change in DAS28 score, adjusted for baseline DAS (data in Table 3 are also adjusted for baseline platelet number). The sign of the coefficient represent the direction of the effect. For example, a coefficient of −1.6 means that higher expression is associated with better response. Every 2-fold increase in gene expression value corresponds to a further reduction on DAS score by 1.6 unit. Likewise, a positive coefficient indicates that higher expression value is associated with poorer response (higher DAS28 score). Table 1 show biomarkers in which the baseline expression (i.e., level prior to undergoing treatment with an IL-6R antibody such as tocilizumab) of a biomarker is predictive for a therapeutic response. Thus, for example, the level of a gene expression product encoded by a gene set forth in Table 1 can be determined in a peripheral blood sample obtained from a rheumatoid arthritis patient. A biomarker positive/negative groups is defined using a threshold in gene expression level. The exact thresholds for each marker can be determined using algorithms well known in the art and will depend on the particular platform and assay used and the desired performance parameters, e.g., sensitivity, specificity, of the assay.

For example, a patient is determined to be likely to exhibit a therapeutic response, or not to exhibit a therapeutic response to the IL-6 antagonizing agent, e.g., tocilizumab, if the level of expression of a biomarker in Table 1 is either above (predicted to exhibit a positive therapeutic response) or below (predicted to the not exhibit a positive therapeutic response) a threshold.

Measurement of the level of expression of a gene set forth in Table 2 also provides the ability to measure the likelihood of a patient to respond to treatment with an IL6-R antagonist, e.g., an IL-6R antibody such as tocilizumab, at later time points. For example, measurement of the expression of a gene set forth in Table 2 is made at base line and, e.g., at 8 weeks following treatment. The change in gene expression between the two measurements is used to calculate likelihood of response at a later time point, such as 16 or 24 weeks. Here again, a threshold of change in response may be applied.

Alternatively, a measurement can be made after initiation of treatment, e.g., at week 8, and an observed normalization' of a level of gene expression against a predetermined value may be used to make the response predication.

Gene expression can also be evaluated for genes listed in Table 5. Each of columns A-J of Table 5 represent genes that were analyzed for the clinical response noted in the column head.

The top 100 genes for ACR are listed in the table with the rank>0. If the value is 0, the gene is not selected for ACR. For each column at least two, typically most, or all of the genes indicated with a value>0 can be analyzed. The gene expression values are used as a linear combination of expression signals from multiple genes in order to predict the classification of clinical response as outlined in the Examples section of ‘class index's’ in the description relating to Table 5. The cutoffs for these linear combinations of gene expression levels are determined by classification algorithms known in the art, such as support vector machines (SVM) (see, e.g., Vapnik, The Nature of Statistical Learning, Springer, N.Y., 1995; Cristianini & Shawe-Taylor, An Introduction to Support Vector Machines, Cambridge University Press, Cambridge, UK, 2000.)

The methods of the invention typically involve recording the level of a gene expression product associated with a beneficial therapeutic outcome, or a negative therapeutic outcome, in a rheumatoid arthritis patient treated with an IL-6R antibody such as tocilizumab. This information may be stored in a computer readable form. Such a computer system typically comprises major subsystems such as a central processor, a system memory (typically RAM), an input/output (I/O) controller, an external device such as a display screen via a display adapter, serial ports, a keyboard, a fixed disk drive via a storage interface and a floppy disk drive operative to receive a floppy disc, and a CD-ROM (or DVD-ROM) device operative to receive a CD-ROM. Many other devices can be connected, such as a network interface connected via a serial port.

The computer system also be linked to a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The computer system can comprise code for interpreting the results of an expression analysis evaluating the baseline level of one or more gene expression products encoded by a gene noted in Table 1. Thus in an exemplary embodiment, the expression analysis results are provided to a computer where a central processor executes a computer program for determining the propensity for a therapeutic response to treatment with an IL-6 receptor antibody.

The invention also provides the use of a computer system, such as that described above, which comprises: (1) a computer; (2) a stored bit pattern encoding the expression results obtained by the methods of the invention, which may be stored in the computer; (3) and, optionally, (4) a program for determining the likelihood for a positive therapeutic response.

The invention further provides methods of generating a report based on the detection of gene expression products in a patient that has rheumatoid arthritis. Such a report is based on the detection of gene expression products encoded by the genes set forth in Table 1 that are associated with either a positive or negative therapeutic outcome.

A patient that has an increased likelihood of having a positive therapeutic response to treatment with IL-6R antibody has at least one gene expression product in Table 1 that is associated with a positive therapeutic response. Typically such a patient has an expression pattern where at least two products encoded by a gene set forth in Table 1 are determined. In some embodiments, the patient may be evaluated for expression levels of products encoded by 3, 4, 5, 6, 7, 8, 9, or 10 or more of the genes set forth in Table 1.

### EXAMPLES

### Example 1

### Analysis of Gene Expression Profiles of Rheumatoid Arthritis Patients Treated with Tocilizumab

Analysis of Gene Expression Data for Association with Response to Change in DAS28 Score.

RNA samples collected from patients with active RA dosed with 8 mg/Kg tocilizumab as a monotherapy in the AMBITION study (Jones, et al., Ann Rheum Dis 2 69:88-96, 2010) were collected at baseline and at week 8 post dose. Two hundred and nine samples (113 baseline samples and 96 “week 8” samples) underwent gene expression profiling through use of an Affymetrix GeneChip® Human Genome U133 Plus 2.0 Array.

After a number of quality control steps on the gene expression data, 2 samples were highlighted as having lower quality, and 207 samples were subjected to further analysis.

The Affymetrix RMA algorithm was used in generating the normalized gene expression data for further analysis. Only probesets with high expression levels (max>4) and those with larger dynamic range (max-min>2) were included. The max and min were taken over all samples. Linear regression was performed for the following analyses. In all analyses, change in Disease Activity Score 28 (DAS28) at week 16 (cDAS28) was used as response endpoint. Week 16 was chosen because it was the earliest time point for escape therapy in the most tocilizumab clinical trials). Baseline DAS was used as a covariate in all analysis since it has significant effect on cDAS.

1. Baseline gene expression versus cDAS28. 111 subjects were included in the analysis.
2. Linear Regression with LASSO Variable Selection using baseline expression data. This is a multivariate analysis method that include all probesets in the model, with L1 penalty on the coefficients of the probesets added to the objective function. (Tibshirani, R. (1996). J. Royal. Statist. Soc B., Vol. 58(1): 267-288)). A subset of the probesets was selected by the model. The number of probesets selected by the model depends on the level of penalty. The optimal level of penalty, which subsequently determined optimal number of probesets selected to achieve the best prediction, was determined using 10-fold cross validation.
3. Change in gene expression at week 8 versus cDAS28. Ninety four subjects were included in the analysis.
4. Linear Regression with LASSO Variable Selection using change in gene expression
5. Baseline gene expression versus cDAS28, adjusting for baseline platelets

Analysis (1) identified a number of probesets that represented activated platelet expressed genes e.g. ITGA2B (CD41), ITGB3 (CD61), JAM3 were present at the top of the list of data ordered by p-value (see, Table 1). There is a correlation of expression of these genes with cDAS28.

This observation prompted a regression analysis of baseline platelet count against change in DAS28. The analysis demonstrated a modest but statistically significant link to baseline platelet count. A far stronger effect size is noted through the correlation of ITGA2B, ITGB3, JAM3 to cDAS28, suggesting that markers of platelet activation are a better predictors of response than platelet count alone.

From analysis (1), it was determined that baseline expression levels of EPHB2 (Ephrin receptor B2) has a correlation to cDAS28. EPHB2 transduces signals that regulate cell attachment and migration and is expressed at higher levels in synovial fibroblasts and exudate lymphocytes in RA, than in those from OA. It's ligand, EphrinB1, is expressed at levels higher in RA peripheral blood lymphocytes (PBL) than healthy controls.

Recombinant EphrinB1 stimulates normal PBL's to exhibit enhanced migration and TNF production, and RA synovial cells to produce IL-6. These results indicate that it is also a useful biomarker for predicting response to tocilizumab.

We reasoned that the high correlation of platelet expressed genes with cDAS observed in analysis (1) could be ‘masking’ the identification of other important response signals. Baseline correction of platelet number in the regression model was therefore performed. From this analysis, ordered by p-value 3 out of 4 components of the NALP1 inflammasome were identified. Inflammasomes are multi-protein cytoplasmic complexes that mediate activation of pro-inflammatory caspases. The NALP1 inflammasome activates caspase 1 and caspase 5. Caspase 1 cleaves pro-IL-1β to IL-1β, and also activates IL-18 and potentially IL-33. We also identified the association of baseline expression of CARD16, a negative regulator of Caspase 1, and the baseline expression of IL-1 receptor, with cDAS. Serum levels of IL1B/IL-18/IL-33 and gene expression signature of transcripts identified above also may be used as biomarkers to predict response to tocilizumab.

From analysis (3), a number of transcripts have been identified that may be used to predict response through change in gene expression 8 weeks from tocilizumab administration. (Table 2). These include caspase 1, a link to the IL-1β/IL-18/IL-33 pathway (and see (4) above), serine palmitoyltransferase, long chain base subunit 2, a link to de novo sphingolipid synthesis of molecules such ceramide and sphingosine-1-phosphate (SIP), and platelet expressed genes such as CD41, CD61, and JAM3.

Lasso variable selection multivariate methodology (analyses 2 and 4) allows identification of transcripts that each contribute a different ‘component’ to the prediction of response. An optimal number of probesets (n=12 and n=13 respectively) were determined by 10 fold cross validation. This analysis identified a number of genes that may be used as predictive biomarkers.

The list of probesets/genes identified by these analyses are shown in Table 1. Table 1 cDASvs.bExp contains probesets/genes whose baseline expression is predictive of tocilizumab treatment response. This list consists of 95 probesets, 12 of which were unmapped, the remaining probesets mapped to 72 unique gene symbols. Among the probesets, 88 were identified by univariate linear regression (analysis 1) and 12 were identified using the multivariate LASSO analysis (analysis 2), with 5 probesets identified by both analyses.

Table 2 cDASvs.cEXP contains probeset/gene expression change from baseline to week 8 that is predictive of tocilizumab treatment response. This list consists of 104 probesets, 6 of which were unmapped, the remaining mapped to 92 unique genes symbols. Among the probesets, 97 were identified by univariate linear regression analysis (analysis 3) and 13 were identified using the multivariate LASSO analysis (analysis 4), with 6 probesets identified by both analyses.

Table 3 (cDASvs.bEXP.AdjustforPlatelet) contains probeset/genes whose baseline expression, combined with baseline platelet count, is predictive of tocilizumab treatment response. This list consists of 81 probesets, 10 of which were unmapped, the remaining mapped to 61 unique genes symbols. All of the probesets were identified by univariate linear regression analysis (analysis 5).

All of the biomarkers may be used univariately or in combination in a multivariate model.

### Example 2

### Identification of Groups of Probesets with Predicative Value for Extreme Response to Tocilizumab

An analysis to identify groups of probesets with predictive value of extreme response to tocilizumab, namely ACR response and EULAR response, was also undertaken.

Two hundred nine CEL files (Affymetrix expression data files) were generated for patients treated with tocilizumab. Two CEL files were excluded from the dataset for technical reasons. One hundred eleven of the remaining 207 CEL files are for the samples at the baseline. This example is focused on the dataset N111.

We considered the four classes of American College of Rheumatology (ACR) response are shown in Table 4.

TABLE 4

| ClassIndex | ACR20 | ACR50 | ACR70 |
|---|---|---|---|
| 1 | 0 | 0 | 0 |
| 2 | 1 | 0 | 0 |
| 3 | 1 | 1 | 0 |
| 4 | 1 | 1 | 1 |

We also considered 3 classes of European League Against Rheumatism (EULAR) response at week 16 (1 for no response, 2 for moderate and 3 for good response). Change in DAS28 at beginning and DAS28 at week 16 (“dDAS28” or “cDAS28”), as well as DAS28 at week 16 was also evaluated. There is one missing data point in DAS28, we therefore have a dataset N110 for DAS28 at week 16 and cDAS28.

For DAS28 at week 16, we define C1 as the class with DAS28 value x>=4 (non response), C2 as the class with x in the range of 2.6 to 4, and C3 as the class with x<2.6 (good response). For ΔDAS28, we define C1 as the class with ΔDAS28 value y<=2.5 (poor response), C2 as the class with y in the range of 2.5 to 3.6, and C3 as the class with y>3.6 (good response).

In all the above class assignments, C1 represents the group with poor response and C4 (ACR) or C3 (other indicators) for good response. C2 (or C2 and C3 for ACR) is the class of moderate response.

### Approaches for Probeset Selection

For each indicator (ACR, EULAR, ΔDAS28, and DAS28 at week 16), we used Dn3 expression signals (see Liu, et al., J. Theortical Biol 243:273-278, 2006; and pending U.S. application Ser. No. 12/578,417) and two different ways of grouping. One grouping is the poor response class versus others (good and moderate response classes). The other grouping is to use only the extreme classes (poor response class versus the good response classes). The sample sizes for the first grouping method are given before, N111 or N110. The sample sizes for the grouping of extreme classes are N62 (ACR), N45 (EULAR), N70 (DAS28 at week 16) and N80 (ΔDAS28).

Dn3 signals (with improvements on MASS using differences of perfect match and mismatch intensities) are typically robust for classification results. For completeness, we also included the probe sets selected with Pn3 signals (using only perfect match intensities and similar to RMA in certain sense).

For each grouping method, we calculated the absolute values of t-statistics and selected the top 100 probe sets with highest absolute values of t-statistics. Their union for 4 different indicators, 2 different signals and 2 different grouping methods (total 8 groups) contains 628 probesets and are listed in Table 5. (For “union of the four different indicators, the 4 different indicators (or 4 different types of responses) are ACR, EULAR, DAS and cDAS. The union is the combination of all probe sets without counting the replicated ones. For example, if set 1 is {1, 3, 5, 7, 9}, set 2 is {1, 2, 3, 4}, Set 3 is {3, 5}, set 4 is {9, 10, 11}, then the union of these 4 sets is {1, 2, 3, 4, 5, 7, 9, 10, 11}).

### Table 5 Description

In Table 5, the first column “N1:54630” lists the 1-based indices in the list of 54630 probe sets targeting human genes on the HG-U133 Plus 2.0 microarray. The second column “ID” lists the Affymetrix probe set IDs.

The next 8 columns provide the ranks of 8 groups of probesets and the information whether a probe set is selected in a particular group. The column names are indicator name, sample size, and signals (Dn3). The value 0 means the probe set is not selected in a particular group. The values 1 through 100 give the ranks of the selected probe sets, where 1 is the top (most significant) one.

The column “AverageScore” provides a score for the summary of the previous 8 columns. The value 0 has no contribution to the score (i.e., the score is 0). For all other values (1 through 100), we calculated (101−value) (so the difference is in the range 1 through 100, but in the reverse order, the largest difference, 100, corresponds to the most significant rank 1). We calculated the average score for the 8 columns and list all average scores in the column. In general, the higher the score, the more significant a probeset for all groups.

The columns “Gene Symbol” and “Gene Title” provide annotations from Affymetrix web site for the selected probe sets.

For Table 5, each group of genes identified in columns C-J of table 5 may be used to form one or more linear combinations of expression signals from multiple genes in order to predict the clinical response as outlined in the description of ‘class index's’ in lines 0080-0084. The cutoffs for these linear combinations of gene expression levels will be determined by classification algorithms such as support vector machines (SVM, The Nature of Statistical Learning, Springer, N.Y., 1995; Cristianini and Shawe-Taylor, An Introduction to Support Vector Machines, Cambridge University Press, Cambridge, UK, 2000). For Table 5, each indications shows a number; expression of at least two genes that have a number greater than 0 can be used (within the same column).

Examples 3 and 4 below provide example of how two and three gene transcripts are used to predict patient response to treatment with an IL-6R antagonist, such as an IL-6R antibody, e.g., tocilizumab. As understood in the art, a multivariate model can be employed that involves additional genes identified herein, e.g., probe sets corresponding to those set forth in Table 1, Table 2, or Table 3.

### Example 3

### Combination on Three Probesets for Predicting the Response Level

Gene transcripts in patient baseline blood samples are measured using Affymetrix human genome U133 plus v2 array. The raw data file are normalized against the data from a set of reference samples from which the algorithm was derived. Expression at the gene transcript level (RMA type of data) will be extracted, in this example, for at the three probesets 12345_at, 12346_at and 12347_at (denoted as e1, e2 and e3) and used in a linear model to give predictions of the week 24 change from baseline DAS28 score (cDAS) if the patient undergoes tocilizumab (TCZ) treatment at 8 mg/kg in combination with methotrexate (MTX).

For TCZ treatment: cDAS=a0*DAS_baseline+a1*e1+a2*e2+a3*e3

The predicted mean change in DAS for the patients will be from 1 to −7, depending on the baseline DAS and gene expression values of e1, e2 and e3. If the patient were to undergo treatment with MTX alone, the predicted mean change in DAS given by:

For MTX treatment: cDAS=b0*DAS_baseline

The predicted mean change is DAS will be from 0 to −3, depending on the patient baseline DAS alone

The treatment choice for each patient is then made based on the difference of these predictions. For example, if patient A has a predicted change in DAS of −4.5 on tocilizumab, and −2 on MTX, the doctor may recommend TCZ treatment. Patient B has the predicted change in DAS of −3 on TCZ and −2.5 on MTX, the doctor may recommend treatment with MTX, as the small additional therapeutic benefit may be not worth the additional cost and any potential risk.

### Example 4

### Combination of Two Transcripts to Predict Patient Response to Treatment

Expression levels of two genes in patient baseline blood samples are measured using quantitative PCR (qPCR). The relative expression levels are represented by ACT. Biomarker groups are defined as following:

Positive: a1*ΔCT1+a2*ΔCT2>=2.1

Negative: a1*ΔCT1+a2*ΔCT2<2.1

Biomarker positive patients are likely to have better response rate compared with biomarker negative patients under tocilizumab treatment, (ACR50 response rate of 55% vs. 38%), while both group have similar response rate when treated with methotrexate, with ACR50 response rate of 35%.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1

| | | gene. | | co- | raw. | exp. | exp. | exp. | | |
|---|---|---|---|---|---|---|---|---|---|---|
| probeset | Accession | Symbol | gene. title | efficient | p. value | median | min | max | Diff | LASSO |
| 240934_at | AI801975 | PIP5K1B | Phosphatidylinositol-4-phosphate | −1.63 | 1.4E−04 | 3.51 | 2.16 | 4.93 | 2.77 | |
| | | | 5-kinase type-1 beta | | | | | | | |
| 231721_at | AF356518 | JAM3 | junctional adhesion molecule 3 | −0.67 | 2.5E−04 | 4.10 | 2.43 | 6.17 | 3.75 | Y |
| 1558938_at | BC043574 | — | — | 1.04 | 2.6E−04 | 4.45 | 3.09 | 5.77 | 2.68 | |
| 206494_s_at | NM_000419 | ITGA2B | integrin, alpha 2b (platelet glycoprotein | −1.00 | 2.8E−04 | 3.97 | 2.64 | 5.76 | 3.12 | |
| | | | IIb of IIb/IIIa complex, antigen CD41) | | | | | | | |
| 216956_s_at | AF098114 | ITGA2B | integrin, alpha 2b (platelet glycoprotein | −0.68 | 4.1E−04 | 4.45 | 2.94 | 6.40 | 3.46 | |
| | | | IIb of IIb/IIIa complex, antigen CD41) | | | | | | | |
| 212811_x_at | AI889380 | SLC1A4 | solute carrier family 1 (glutamate/ | 1.07 | 5.6E−04 | 3.91 | 2.59 | 4.86 | 2.27 | |
| | | | neutral amino acid transporter), | | | | | | | |
| | | | member 4 | | | | | | | |
| 209589_s_at | AF025304 | EPHB2 | EPH receptor B2 | −0.90 | 7.1E−04 | 3.37 | 2.11 | 5.25 | 3.15 | |
| 234618_at | AL049434 | PHTF1 | Putative homeodomain transcription | 0.93 | 9.2E−04 | 2.54 | 1.79 | 4.40 | 2.61 | |
| | | | factor 1 | | | | | | | |
| 239274_at | AV729557 | PICALM | Phosphatidylinositol-binding clathrin | 1.13 | 9.7E−04 | 6.10 | 5.00 | 7.13 | 2.13 | |
| | | | assembly protein | | | | | | | |
| 217876_at | NM_012087 | GTF3C5 | general transcription factor IIIC, | −1.22 | 1.2E−03 | 4.24 | 3.18 | 5.23 | 2.05 | |
| | | | polypeptide 5, 63 kDa | | | | | | | |
| 240980_at | R61819 | — | — | 1.25 | 1.3E−03 | 2.22 | 1.58 | 4.29 | 2.71 | |
| 214364_at | W84525 | MTERFD2 | MTERF domain containing 2 | −1.21 | 1.3E−03 | 3.30 | 2.00 | 4.62 | 2.61 | |
| 209006_s_at | AF247168 | C1orf63 | chromosome 1 open reading frame 63 | 1.08 | 1.5E−03 | 5.89 | 4.66 | 7.63 | 2.97 | |
| 234948_at | AK026640 | SLC27A5 | solute carrier family 27 (fatty acid | −1.19 | 1.6E−03 | 3.73 | 2.92 | 4.97 | 2.05 | |
| | | | transporter), member 5 | | | | | | | |
| 204626_s_at | J02703 | ITGB3 | integrin, beta 3 (platelet glycoprotein | −0.51 | 1.7E−03 | 7.30 | 4.89 | 9.40 | 4.51 | Y |
| | | | IIIa, antigen CD61) | | | | | | | |
| 219476_at | NM_024115 | C1orf116 | chromosome 1 open reading frame 116 | −1.04 | 1.9E−03 | 2.39 | 1.70 | 4.31 | 2.61 | |
| 206493_at | NM_000419 | ITGA2B | integrin, alpha 2b (platelet | −0.56 | 2.0E−03 | 7.45 | 5.05 | 9.56 | 4.51 | |
| | | | glycoprotein IIb of IIb/IIIa complex, | | | | | | | |
| | | | antigen CD41) | | | | | | | |
| 239714_at | AA780063 | — | — | −1.11 | 2.1E−03 | 3.41 | 2.55 | 4.80 | 2.25 | |
| 217179_x_at | X79782 | — | — | 0.90 | 2.2E−03 | 4.56 | 3.83 | 6.66 | 2.83 | |
| 225685_at | AI801777 | — | — | 0.99 | 2.6E−03 | 6.32 | 5.29 | 7.46 | 2.17 | |
| 1552309_a_at | NM_144573 | NEXN | nexilin (F actin binding protein) | 0.69 | 2.6E−03 | 3.63 | 1.94 | 5.33 | 3.39 | |
| 232472_at | AK022461 | FNDC3B | Fibronectin type III domain-containing | 0.69 | 2.7E−03 | 3.86 | 2.60 | 5.60 | 3.00 | |
| | | | protein 3B | | | | | | | |
| 229643_at | AI857933 | ITGA6 | Integrin alpha 6B [human, mRNA | −0.98 | 2.7E−03 | 3.75 | 2.86 | 5.20 | 2.34 | |
| | | | Partial, 528 nt] | | | | | | | |
| 238080_at | BF195052 | B4GALNT4 | beta-1,4-N-acetyl-galactosaminyl | −1.06 | 2.7E−03 | 3.13 | 2.28 | 4.47 | 2.19 | |
| | | | transferase 4 | | | | | | | |
| 243187_at | AA888821 | PVRL2 | Poliovirus receptor-related protein | 0.88 | 2.9E−03 | 2.25 | 1.48 | 4.09 | 2.61 | |
| | | | 2 Precursor | | | | | | | |
| 208792_s_at | M25915 | CLU | clusterin | −0.55 | 3.0E−03 | 6.46 | 4.57 | 8.51 | 3.94 | |
| 208593_x_at | NM_004382 | CRHR1 | corticotropin releasing hormone | −1.18 | 3.5E−03 | 3.24 | 2.25 | 4.28 | 2.04 | |
| | | | receptor 1 | | | | | | | |
| 217472_at | J02963 | — | — | −0.84 | 3.7E−03 | 3.98 | 2.98 | 5.66 | 2.68 | |
| 243106_at | AA916861 | CLEC12A | C-type lectin protein CLL-1 | 0.28 | 3.9E−03 | 3.93 | 1.90 | 7.11 | 5.22 | Y |
| 225680_at | BE896303 | LRWD1 | leucine-rich repeats and WD repeat | −1.10 | 3.9E−03 | 5.25 | 4.35 | 7.01 | 2.66 | |
| | | | domain containing 1 | | | | | | | |
| 212613_at | AI991252 | BTN3A2 | butyrophilin, subfamily 3, member A2 | 0.55 | 4.0E−03 | 6.00 | 2.43 | 6.98 | 4.56 | |
| 230888_at | AW300278 | WDR91 | CDNA FLJ23886 fis, clone LNG13909 | 0.76 | 4.0E−03 | 2.73 | 1.49 | 4.31 | 2.82 | |
| 212592_at | AV733266 | IGJ | immunoglobulin J polypeptide, linker | 0.32 | 4.0E−03 | 3.38 | 1.38 | 8.41 | 7.03 | Y |
| | | | protein for immunoglobulin | | | | | | | |
| | | | alpha and mu polypeptides | | | | | | | |
| 216145_at | AL137713 | — | — | −1.19 | 4.3E−03 | 2.81 | 2.20 | 4.25 | 2.05 | |
| 235971_at | AI147211 | — | — | 0.71 | 4.4E−03 | 3.59 | 2.63 | 5.66 | 3.04 | |
| 1562743_at | BC042873 | ZNF33B | Zinc finger protein 33B (ZNF33B), | −1.03 | 4.4E−03 | 3.62 | 2.32 | 4.74 | 2.42 | |
| | | | mRNA | | | | | | | |
| 208791_at | M25915 | CLU | clusterin | −0.53 | 4.6E−03 | 5.47 | 3.72 | 7.37 | 3.65 | |
| 222411_s_at | AW087870 | SSR3 | signal sequence receptor, gamma | 0.87 | 4.8E−03 | 5.55 | 4.42 | 6.82 | 2.40 | |
| | | | (translocon-associated protein gamma) | | | | | | | |
| 212813_at | AA149644 | JAM3 | junctional adhesion molecule 3 | −0.75 | 4.9E−03 | 5.14 | 4.07 | 6.61 | 2.54 | |
| 225831_at | AW016830 | LUZP1 | leucine zipper protein 1 | −1.74 | 5.0E−03 | 4.16 | 3.58 | 6.99 | 3.41 | |
| 232079_s_at | BE867789 | PVRL2 | poliovirus receptor-related 2 | 0.45 | 5.0E−03 | 3.15 | 2.33 | 6.76 | 4.42 | |
| | | | (herpesvirus entry mediator B) | | | | | | | |
| 202112_at | NM_000552 | VWF | von Willebrand factor | −0.72 | 5.1E−03 | 3.34 | 2.31 | 5.50 | 3.20 | |
| 231057_at | AU144266 | MTMR2 | Myotubularin-related protein 2 | 1.11 | 5.3E−03 | 2.91 | 2.07 | 4.22 | 2.15 | |
| 220476_s_at | NM_019099 | C1orf183 | chromosome 1 open reading frame 183 | −0.90 | 5.5E−03 | 5.53 | 4.21 | 6.32 | 2.11 | |
| 232726_at | AK024956 | MAML3 | Mastermind-like protein 3 | 0.75 | 5.5E−03 | 3.71 | 2.61 | 4.94 | 2.33 | |
| 1552398_a_at | NM_138337 | CLEC12A | C-type lectin domain family 12, | 0.31 | 5.7E−03 | 5.84 | 4.09 | 8.70 | 4.61 | |
| | | | member A | | | | | | | |
| 238183_at | AI632259 | PRKAR1B | cAMP-dependent protein kinase | −0.60 | 6.0E−03 | 5.73 | 3.24 | 7.26 | 4.02 | |
| | | | type I-beta regulatory subunit | | | | | | | |
| 231174_s_at | H92979 | — | — | 0.83 | 6.2E−03 | 2.00 | 1.11 | 4.39 | 3.28 | |
| 203545_at | NM_024079 | ALG8 | asparagine-linked glycosylation 8, | 0.72 | 6.6E−03 | 3.73 | 2.08 | 5.29 | 3.21 | |
| | | | alpha-1,3-glucosyltransferase homolog | | | | | | | |
| | | | (S. cerevisiae) | | | | | | | |
| 227551_at | BE856596 | FAM108B1 | family with sequence similarity 108, | 0.77 | 6.8E−03 | 3.98 | 2.17 | 5.30 | 3.13 | |
| | | | member B1 | | | | | | | |
| 229530_at | BF002625 | GUCY1A3 | guanylate cyclase 1, soluble, alpha 3 | −0.62 | 6.9E−03 | 3.07 | 1.99 | 4.73 | 2.74 | |
| 233852_at | AK025631 | POLH | polymerase (DNA directed), eta | 0.85 | 6.9E−03 | 4.78 | 3.86 | 6.77 | 2.91 | |
| 231720_s_at | AF356518 | JAM3 | junctional adhesion molecule 3 | −0.78 | 7.0E−03 | 4.48 | 3.49 | 5.99 | 2.50 | |
| 218435_at | NM_013238 | DNAJC15 | DnaJ (Hsp40) homolog, subfamily C, | 0.64 | 7.0E−03 | 5.15 | 3.55 | 6.54 | 2.99 | |
| | | | member 15 | | | | | | | |
| 202874_s_at | NM_001695 | ATP6V1C1 | ATPase, H+ transporting, lysosomal | 0.73 | 7.2E−03 | 5.48 | 4.00 | 6.98 | 2.98 | |
| | | | 42 kDa, V1 subunit C1 | | | | | | | |
| 244308_at | BF514096 | SYT15 | Chr10 synaptotagmin (CHR10SYT | 0.73 | 7.3E−03 | 2.48 | 1.57 | 5.12 | 3.55 | |
| | | | gene) | | | | | | | |
| 238589_s_at | AW601184 | ATXN2 | Ataxin-2 | 0.67 | 7.3E−03 | 4.82 | 3.32 | 6.35 | 3.03 | |
| 203064_s_at | NM_004514 | FOXK2 | forkhead box K2 | 0.79 | 7.5E−03 | 4.33 | 3.01 | 7.04 | 4.03 | |
| 231886_at | AL137655 | DKFZP434 | similar to hypothetical protein | 0.50 | 7.6E−03 | 4.49 | 2.87 | 6.21 | 3.33 | |
| | | B2016 | LOC284701 | | | | | | | |
| 221942_s_at | AI719730 | GUCY1A3 | guanylate cyclase 1, soluble, alpha 3 | −0.52 | 7.6E−03 | 2.75 | 1.40 | 4.54 | 3.14 | |
| 1564155_x_at | BC041466 | — | — | 0.61 | 7.7E−03 | 4.06 | 2.58 | 5.71 | 3.13 | |
| 228040_at | AW294192 | MGC21881 | hypothetical locus MGC21881 | 0.77 | 7.7E−03 | 3.31 | 2.13 | 5.03 | 2.90 | |
| 207500_at | NM_004347 | CASP5 | caspase 5, apoptosis-related cysteine | 0.60 | 7.8E−03 | 3.89 | 2.30 | 6.14 | 3.85 | |
| | | | peptidase | | | | | | | |
| 211637_x_at | L23516 | IGH | immunoglobulin heavy locus | 0.59 | 8.1E−03 | 5.36 | 4.09 | 7.61 | 3.52 | |
| 232030_at | AK023817 | KIAA1632 | KIAA1632 | 0.61 | 8.1E−03 | 2.31 | 1.30 | 4.36 | 3.06 | |
| 210219_at | U36501 | SP100 | SP100 nuclear antigen | 0.65 | 8.2E−03 | 1.67 | 1.10 | 6.37 | 5.28 | |
| 209610_s_at | BF340083 | SLC1A4 | solute carrier family 1 (glutamate/ | 0.64 | 8.2E−03 | 2.76 | 1.53 | 4.32 | 2.78 | |
| | | | neutral amino acid transporter), | | | | | | | |
| | | | member 4 | | | | | | | |
| 1558120_at | BE379787 | DDX3X | DEAD (Asp-Glu-Ala-Asp) box | 0.89 | 8.4E−03 | 4.10 | 2.82 | 5.14 | 2.32 | |
| | | | polypeptide 3, X-linked | | | | | | | |
| 210127_at | BC002510 | RAB6B | RAB6B, member RAS oncogene family | −0.46 | 8.5E−03 | 3.49 | 2.15 | 5.71 | 3.56 | |
| 210456_at | AF148464 | PCYT1B | phosphate cytidylyltransferase 1, | −0.88 | 8.5E−03 | 3.93 | 3.07 | 5.19 | 2.12 | |
| | | | choline, beta | | | | | | | |
| 1559810_at | BF724577 | LOC642313 | hypothetical LOC642313 | 0.79 | 8.6E−03 | 2.91 | 1.56 | 4.71 | 3.15 | |
| 209651_at | BC001830 | TGFB1I1 | transforming growth factor beta 1 | −0.48 | 8.7E−03 | 1.55 | 0.91 | 5.59 | 4.68 | |
| | | | induced transcript 1 | | | | | | | |
| 239442_at | BF589179 | CEP68 | centrosomal protein 68 kDa | 0.86 | 8.7E−03 | 5.28 | 4.08 | 6.17 | 2.09 | |
| 1558742_at | BE899474 | DEXI | Dexamethasone-induced protein | 0.74 | 8.7E−03 | 2.98 | 1.67 | 4.76 | 3.09 | |
| 238894_at | AW665144 | RABGAP1L | RAB GTPase-activating protein 1-like | 0.80 | 8.8E−03 | 3.76 | 2.59 | 5.04 | 2.45 | |
| 209846_s_at | BC002832 | BTN3A2 | butyrophilin, subfamily 3, member A2 | 0.40 | 8.8E−03 | 6.65 | 1.82 | 7.73 | 5.91 | Y |
| 215093_at | U82671 | NSDHL | NAD(P) dependent steroid | 0.53 | 8.9E−03 | 3.50 | 1.82 | 6.28 | 4.46 | |
| | | | dehydrogenase-like | | | | | | | |
| 213814_s_at | AA741303 | SNTB2 | CDNA clone IMAGE: 5263917 | −0.69 | 9.2E−03 | 4.24 | 2.90 | 5.32 | 2.42 | |
| 219348_at | NM_018467 | USE1 | unconventional SNARE in the ER 1 | −1.11 | 9.5E−03 | 6.10 | 5.30 | 8.00 | 2.70 | |
| | | | homolog (S. cerevisiae) | | | | | | | |
| 240482_at | AI955094 | HDAC3 | Histone deacetylase 3 (HD3) | 0.74 | 9.6E−03 | 5.31 | 3.99 | 6.47 | 2.49 | |
| 201058_s_at | NM_0066097 | MYL9 | myosin, light chain 9, regulatory | −0.44 | 9.6E−03 | 5.68 | 3.84 | 8.28 | 4.44 | |
| 225900_at | AW294630 | EXOC6B | exocyst complex component 6B | 0.73 | 9.7E−03 | 3.23 | 1.91 | 5.03 | 3.12 | |
| 230788_at | BF059748 | GCNT2 | glucosaminyl (N-acetyl) transferase | 0.46 | 9.8E−03 | 3.91 | 2.57 | 6.90 | 4.34 | |
| | | | 2, I-branching enzyme (I blood group) | | | | | | | |
| 211368_s_at | U13700 | CASP1 | caspase 1, | 0.73 | 9.8E−03 | 6.41 | 4.86 | 7.73 | 2.86 | |
| | | | apoptosis-related cysteine peptidase | | | | | | | |
| | | | (interleukin 1, beta, convertase) | | | | | | | |
| 235066_at | AI078534 | MAP4 | microtubule-associated protein 4 | −1.25 | 9.8E−03 | 2.81 | 2.21 | 4.25 | 2.03 | |
| 206176_at | NM_001718 | BMP6 | bone morphogenetic protein 6 | −0.72 | 9.9E−03 | 3.27 | 2.13 | 4.85 | 2.73 | |
| 232078_at | BE867789 | PVRL2 | poliovirus receptor-related 2 | 0.38 | 9.9E−03 | 2.53 | 1.29 | 6.24 | 4.95 | |
| | | | (herpesvirus entry mediator B) | | | | | | | |
| 211026_s_at | BC006230 | MGLL | monoglyceride lipase | −0.75 | 1.0E−02 | 3.28 | 2.30 | 5.05 | 2.74 | |
| 1557012_a_at | BC040670 | — | — | | | | | | | Y |
| 203911_at | NM_002885 | RAP1GAP | RAP1 GTPase activating protein | | | | | | | Y |
| 209728_at | BC005312 | HLA-DRB4 | major histocompatibility complex, | | | | | | | Y |
| | | | class II, DR beta 4 | | | | | | | |
| 217207_s_at | AK025267 | BTNL3 | butyrophilin-like 3 | | | | | | | Y |
| 220281_at | AI632015 | SLC12A1 | solute carrier family 12 (sodium/ | | | | | | | Y |
| | | | potassium/chloride transporters), | | | | | | | |
| | | | member 1 | | | | | | | |
| 230720_at | AI884906 | RNF182 | ring finger protein 182 | | | | | | | Y |
| 235446_at | AW856618 | — | — | | | | | | | Y |

TABLE 2

| | | | | co- | | exp. | | | | |
|---|---|---|---|---|---|---|---|---|---|---|
| | | gene. | | effi- | raw. | me- | exp. | exp. | | |
| probeset | Accession | symbol | gene. title | cient | p. value | dian | min | Max | diff | LASSO |
| 1562743_at | BC042873 | ZNF33B | Zinc finger protein 33B (ZNF33B), mRNA | 1.18 | 3.1E−04 | 3.62 | 2.32 | 4.74 | 2.42 | |
| 242109_at | AI038577 | SYTL3 | CDNA FLJ61334 complete cds, moderately | −1.01 | 4.3E−04 | 2.83 | 1.90 | 4.43 | 2.53 | |
| | | | similar to Synaptotagmin-like protein 3 | | | | | | | |
| 232354_at | AK022083 | VPS37B | Vacuolar protein sorting-associated protein | −0.88 | 6.1E−04 | 4.36 | 3.04 | 5.60 | 2.55 | |
| | | | 37B | | | | | | | |
| 226865_at | AW130600 | — | — | −1.00 | 8.1E−04 | 5.64 | 3.75 | 6.66 | 2.90 | |
| 224091_at | AF116642 | — | — | −0.82 | 8.7E−04 | 5.38 | 4.30 | 6.69 | 2.39 | |
| 211367_s_at | U13699 | CASP1 | caspase 1, apoptosis-related cysteine | −1.07 | 1.2E−03 | 7.44 | 6.20 | 9.01 | 2.81 | |
| | | | peptidase (interleukin 1, beta, convertase) | | | | | | | |
| 218728_s_at | NM_014184 | CNIH4 | cornichon homolog 4 (Drosophila) | −0.88 | 1.2E−03 | 5.95 | 4.56 | 7.10 | 2.55 | |
| 216203_at | U15555 | SPTLC2 | serine palmitoyltransferase, long chain | −0.93 | 1.3E−03 | 3.00 | 2.23 | 5.29 | 3.07 | |
| | | | base subunit 2 | | | | | | | |
| 219476_at | NM_024115 | C1orf116 | chromosome 1 open reading frame 116 | 0.88 | 1.5E−03 | 2.39 | 1.70 | 4.31 | 2.61 | |
| 233660_at | BG540685 | EHD4 | EH-domain containing 4 | 0.95 | 1.5E−03 | 3.97 | 2.48 | 5.06 | 2.59 | |
| 215431_at | AI033054 | SNTB1 | syntrophin, beta 1 (dystrophin-associated | 1.07 | 1.6E−03 | 3.34 | 2.45 | 4.70 | 2.26 | |
| | | | protein A1, 59 kDa, basic component 1) | | | | | | | |
| 219731_at | NM_024343 | FLJ34077 | weakly similar to zinc finger protein 195 | −0.99 | 1.8E−03 | 5.81 | 4.52 | 7.18 | 2.66 | |
| 241339_at | BF437886 | TTC39B | Tetratricopeptide repeat protein 39B | 0.96 | 1.8E−03 | 4.59 | 3.45 | 5.74 | 2.29 | |
| 211368_s_at | U13700 | CASP1 | caspase 1, apoptosis-related cysteine | −0.82 | 1.9E−03 | 6.41 | 4.86 | 7.73 | 2.86 | |
| | | | peptidase (interleukin 1, beta, convertase) | | | | | | | |
| 209006_s_at | AF247168 | C1orf63 | chromosome 1 open reading frame 63 | −0.88 | 2.0E−03 | 5.89 | 4.66 | 7.63 | 2.97 | |
| 1559469_s_at | BC006013 | SIPA1L2 | signal-induced proliferation-associated 1 | −0.54 | 2.1E−03 | 6.17 | 4.01 | 8.18 | 4.18 | Y |
| | | | like 2 | | | | | | | |
| 239613_at | AA833846 | TMED3 | Transmembrane emp24 domain-containing | 0.83 | 2.2E−03 | 3.20 | 2.39 | 4.56 | 2.17 | |
| | | | protein 3 Precursor | | | | | | | |
| 206494_s_at | NM_000419 | ITGA2B | integrin, alpha 2b (platelet glycoprotein | 0.87 | 2.4E−03 | 3.97 | 2.64 | 5.76 | 3.12 | |
| | | | IIb of IIb/IIIa complex, antigen CD41) | | | | | | | |
| 231721_at | AF356518 | JAM3 | junctional adhesion molecule 3 | 0.57 | 2.6E−03 | 4.10 | 2.43 | 6.17 | 3.75 | Y |
| 227461_at | AA632295 | STON2 | stonin 2 | 0.55 | 2.8E−03 | 2.49 | 1.44 | 4.47 | 3.03 | Y |
| 213810_s_at | AW007137 | AKIRIN2 | CDNA FLJ10342 fis, clone NT2RM2000837 | −0.97 | 2.8E−03 | 5.88 | 4.85 | 7.36 | 2.51 | |
| 211366_x_at | U13698 | CASP1 | caspase 1, apoptosis-related cysteine | −0.96 | 2.8E−03 | 7.42 | 6.32 | 8.70 | 2.37 | |
| | | | peptidase (interleukin 1, beta, convertase) | | | | | | | |
| 209499_x_at | BF448647 | TNFSF12 | tumor necrosis factor (ligand) superfamily, | −0.69 | 2.9E−03 | 4.43 | 2.78 | 5.75 | 2.97 | |
| | | | member 12 | | | | | | | |
| 228040_at | AW294192 | MGC21881 | hypothetical locus MGC21881 | −0.73 | 3.0E−03 | 3.31 | 2.13 | 5.03 | 2.90 | |
| 202254_at | AB007900 | SIPA1L1 | KIAA0440 | −0.81 | 3.4E−03 | 3.72 | 2.19 | 4.88 | 2.69 | |
| 239936_at | AA126428 | DLEU2 | deleted in lymphocytic leukemia 2 | −0.85 | 3.5E−03 | 3.44 | 1.74 | 4.26 | 2.52 | |
| | | | (non-protein coding) | | | | | | | |
| 1570165_at | BC027983 | CHST11 | Carbohydrate sulfotransferase 11 | 0.90 | 3.5E−03 | 3.09 | 1.94 | 4.44 | 2.50 | |
| 232030_at | AK023817 | KIAA1632 | KIAA1632 | −0.64 | 3.7E−03 | 2.31 | 1.30 | 4.36 | 3.06 | |
| 209970_x_at | M87507 | CASP1 | caspase 1, apoptosis-related cysteine | −0.80 | 3.7E−03 | 6.58 | 5.06 | 7.70 | 2.64 | |
| | | | peptidase (interleukin 1, beta, convertase) | | | | | | | |
| 201615_x_at | AI685060 | CALD1 | caldesmon 1 | 0.76 | 3.7E−03 | 5.11 | 3.82 | 6.65 | 2.83 | |
| 238979_at | BE501771 | C10orf33 | chromosome 10 open reading frame 33 | 0.86 | 3.7E−03 | 4.15 | 3.33 | 5.68 | 2.35 | |
| 239824_s_at | BF971873 | TMEM107 | transmembrane protein 107 | 0.81 | 3.7E−03 | 3.01 | 2.19 | 4.57 | 2.38 | |
| 218988_at | NM_018656 | SLC35E3 | solute carrier family 35, member E3 | −0.70 | 3.9E−03 | 4.22 | 3.19 | 5.69 | 2.49 | |
| 221942_s_at | AI719730 | GUCY1A3 | guanylate cyclase 1, soluble, alpha 3 | 0.58 | 3.9E−03 | 2.75 | 1.40 | 4.54 | 3.14 | Y |
| 215739_s_at | AJ003062 | TUBGCP3 | tubulin, gamma complex associated | 0.79 | 3.9E−03 | 3.71 | 1.83 | 4.79 | 2.96 | |
| | | | protein 3 | | | | | | | |
| 223501_at | AW151360 | TNFSF13B | tumor necrosis factor (ligand) superfamily, | −0.60 | 4.0E−03 | 4.50 | 2.96 | 6.73 | 3.77 | |
| | | | member 13b | | | | | | | |
| 204629_at | NM_013327 | PARVB | parvin, beta | 0.76 | 4.1E−03 | 3.92 | 2.54 | 5.41 | 2.86 | |
| 211908_x_at | M87268 | IGHG1 | Immunoglobulin lambda heavy chain | −0.89 | 4.1E−03 | 5.59 | 4.59 | 7.12 | 2.53 | |
| 1554744_at | BC033638 | CARD16 | caspase recruitment domain family, | −0.52 | 4.2E−03 | 4.71 | 3.17 | 7.41 | 4.24 | |
| | | | member 16 | | | | | | | |
| 202270_at | NM_002053 | GBP1 | guanylate binding protein 1, | −0.37 | 4.3E−03 | 3.81 | 1.90 | 6.97 | 5.06 | Y |
| | | | interferon-inducible, 67 kDa | | | | | | | |
| 208593_x_at | NM_004382 | CRHR1 | corticotropin releasing hormone receptor 1 | 0.95 | 4.6E−03 | 3.24 | 2.25 | 4.28 | 2.04 | |
| 1561171_a_at | AK093450 | FLJ36131 | hypothetical protein FLJ36131 | −0.90 | 4.8E−03 | 4.00 | 2.95 | 5.20 | 2.25 | |
| 244752_at | AI563915 | ZNF438 | zinc finger protein 438 | −0.64 | 4.8E−03 | 5.10 | 2.46 | 6.79 | 4.32 | |
| 209651_at | BC001830 | TGFB1I1 | transforming growth factor beta 1 induced | 0.50 | 4.9E−03 | 1.55 | 0.91 | 5.59 | 4.68 | |
| | | | transcript 1 | | | | | | | |
| 235900_at | AW016030 | SPNS3 | spinster homolog 3 (Drosophila) | 0.84 | 5.0E−03 | 3.32 | 2.23 | 5.01 | 2.78 | |
| 235040_at | BG168618 | RUNDC1 | RUN domain containing 1 | 0.55 | 5.0E−03 | 3.36 | 2.10 | 4.98 | 2.88 | |
| 214009_at | R10150 | MSL3 | male-specific lethal 3 homolog (Drosophila) | −1.17 | 5.2E−03 | 4.43 | 3.29 | 5.36 | 2.08 | |
| 226388_at | AI675780 | TCEA3 | transcription elongation factor A (SII), 3 | 0.57 | 5.2E−03 | 4.47 | 2.97 | 6.33 | 3.35 | |
| 232840_at | AK025004 | FNDC3B | Fibronectin type III domain-containing | −0.82 | 5.8E−03 | 5.31 | 4.07 | 6.46 | 2.39 | |
| | | | protein 3B | | | | | | | |
| 227640_s_at | AI492167 | RP9 | retinitis pigmentosa 9 (autosomal dominant) | 0.84 | 5.9E−03 | 4.94 | 3.98 | 6.03 | 2.05 | |
| 244308_at | BF514096 | SYT15 | Chr10 synaptotagmin (CHR10SYT gene) | −0.62 | 6.0E−03 | 2.48 | 1.57 | 5.12 | 3.55 | |
| 232472_at | AK022461 | FNDC3B | Fibronectin type III domain-containing | −0.63 | 6.0E−03 | 3.86 | 2.60 | 5.60 | 3.00 | |
| | | | protein 3B | | | | | | | |
| 1562458_at | AL833723 | UBE2W | ubiquitin-conjugating enzyme E2W | −0.76 | 6.1E−03 | 3.77 | 2.35 | 5.05 | 2.70 | |
| | | | (putative) | | | | | | | |
| 224009_x_at | AF240697 | DHRS9 | dehydrogenase/reductase (SDR family) | −0.48 | 6.1E−03 | 5.12 | 2.97 | 7.43 | 4.46 | |
| | | | member 9 | | | | | | | |
| 228428_at | AA521285 | FAM102A | CDNA FLJ37031 fis, clone BRACE2011199 | 1.01 | 6.1E−03 | 7.54 | 6.36 | 8.44 | 2.08 | |
| 202688_at | NM_003810 | TNFSF10 | tumor necrosis factor (ligand) superfamily, | −0.54 | 6.2E−03 | 6.23 | 4.73 | 8.52 | 3.79 | |
| | | | member 10 | | | | | | | |
| 238669_at | BE613133 | PTGS1 | prostaglandin-endoperoxide synthase 1 | 0.68 | 6.3E−03 | 7.18 | 5.33 | 8.58 | 3.25 | |
| | | | (prostaglandin G/H synthase and | | | | | | | |
| | | | cyclooxygenase) | | | | | | | |
| 234948_at | AK026640 | SLC27A5 | solute carrier family 27 (fatty acid | 0.91 | 6.4E−03 | 3.73 | 2.92 | 4.97 | 2.05 | |
| | | | transporter), member 5 | | | | | | | |
| 223519_at | AW069181 | ZAK | sterile alpha motif and leucine zipper | −0.63 | 6.4E−03 | 2.33 | 1.24 | 4.28 | 3.04 | |
| | | | containing kinase AZK | | | | | | | |
| 206361_at | NM_004778 | GPR44 | G protein-coupled receptor 44 | 0.76 | 6.5E−03 | 5.20 | 3.55 | 7.85 | 4.30 | |
| 227699_at | BF511003 | C14orf149 | chromosome 14 open reading frame 149 | −0.71 | 6.6E−03 | 3.23 | 2.02 | 4.26 | 2.24 | |
| 218167_at | NM_016627 | AMZ2 | archaelysin family metallopeptidase 2 | −0.83 | 6.7E−03 | 5.02 | 3.11 | 5.93 | 2.82 | |
| 1562955_at | BC028181 | WDFY1 | WD repeat and FYVE domain-containing | −0.69 | 6.7E−03 | 3.37 | 2.23 | 4.91 | 2.68 | |
| | | | protein 1 | | | | | | | |
| 236132_at | AI870477 | TLN1 | talin 1 | −0.72 | 6.7E−03 | 4.19 | 3.23 | 6.24 | 3.01 | |
| 1560867_a_at | AF085926 | NEDD9 | Enhancer of filamentation 1 | −0.73 | 6.8E−03 | 2.33 | 1.69 | 4.88 | 3.19 | |
| 1557455_s_at | AF086333 | MOSPD1 | motile sperm domain containing 1 | −0.82 | 6.9E−03 | 3.42 | 2.58 | 5.24 | 2.65 | |
| 201481_s_at | NM_002862 | PYGB | phosphorylase, glycogen; brain | 0.73 | 7.0E−03 | 4.57 | 3.40 | 6.21 | 2.81 | |
| 210904_s_at | U81380 | IL13RA1 | interleukin 13 receptor, alpha 1 | −0.67 | 7.0E−03 | 7.05 | 5.34 | 8.57 | 3.22 | |
| 239467_at | AI806747 | — | — | −0.41 | 7.0E−03 | 2.36 | 0.70 | 5.45 | 4.76 | Y |
| 238428_at | BG542347 | KCNJ15 | potassium inwardly-rectifying channel, | −0.73 | 7.1E−03 | 5.53 | 3.82 | 6.92 | 3.10 | |
| | | | subfamily J, member 15 | | | | | | | |
| 226459_at | AW575754 | PIK3AP1 | phosphoinositide-3-kinase adaptor protein 1 | −0.52 | 7.2E−03 | 6.28 | 4.62 | 7.91 | 3.29 | |
| 1568658_at | BU069195 | C2orf74 | chromosome 2 open reading frame 74 | 0.60 | 7.3E−03 | 3.00 | 1.58 | 4.78 | 3.20 | |
| 201921_at | NW_004125 | GNG10 | guanine nucleotide binding protein | −0.65 | 7.3E−03 | 7.74 | 6.19 | 9.25 | 3.06 | |
| | | | (G protein), gamma 10 | | | | | | | |
| 211254_x_at | AF031549 | RHAG | Rh-associated glycoprotein | −0.88 | 7.5E−03 | 2.50 | 1.70 | 4.32 | 2.62 | |
| 1555338_s_at | AF159174 | AQP10 | aquaporin 10 | 0.73 | 7.8E−03 | 3.09 | 1.86 | 5.46 | 3.61 | |
| 237306_at | AA447558 | ZNF829 | zinc finger protein 829 | 0.73 | 7.8E−03 | 2.07 | 1.33 | 4.11 | 2.78 | |
| 240934_at | AI801975 | PIP5K1B | Phosphatidylinositol-4-phosphate 5-kinase | 0.91 | 8.1E−03 | 3.51 | 2.16 | 4.93 | 2.77 | |
| | | | type-1 beta | | | | | | | |
| 241603_at | BE745453 | ATP11A | ATPase, class VI, type 11A | −0.79 | 8.2E−03 | 5.18 | 3.93 | 6.23 | 2.30 | |
| 1553697_at | NM_145257 | C1orf96 | chromosome 1 open reading frame 96 | −0.58 | 8.2E−03 | 3.63 | 1.96 | 5.89 | 3.93 | |
| 1552701_a_at | NM_052889 | CARD16 | caspase recruitment domain family, | −0.61 | 8.3E−03 | 6.99 | 5.50 | 8.51 | 3.01 | |
| | | | member 16 | | | | | | | |
| 209686_at | BC001766 | S100B | S100 calcium binding protein B | 0.94 | 8.5E−03 | 2.20 | 1.61 | 4.23 | 2.62 | |
| 1555968_a_at | AA362254 | — | — | −0.57 | 8.5E−03 | 4.11 | 2.54 | 5.51 | 2.97 | |
| 241834_at | AW299520 | IPW | imprinted in Prader-Willi syndrome | 0.58 | 8.8E−03 | 3.09 | 1.80 | 5.04 | 3.25 | |
| | | | (non-protein coding) | | | | | | | |
| 230585_at | AI632692 | — | — | −0.47 | 8.9E−03 | 3.84 | 1.87 | 5.75 | 3.88 | |
| 214523_at | NM_001805 | CEBPE | CCAAT/enhancer binding protein | 0.77 | 8.9E−03 | 4.94 | 4.18 | 8.28 | 4.10 | |
| | | | (C/EBP), epsilon | | | | | | | |
| 218204_s_at | NM_024513 | FYCO1 | FYVE and coiled-coil domain containing 1 | 0.72 | 8.9E−03 | 3.45 | 2.28 | 4.54 | 2.27 | |
| 213860_x_at | AW268585 | CSNK1A1 | casein kinase 1, alpha 1 | −0.57 | 9.0E−03 | 5.28 | 3.51 | 6.66 | 3.14 | |
| 213803_at | BG545463 | KPNB1 | Importin subunit beta-1 | −0.68 | 9.1E−03 | 5.96 | 4.71 | 7.02 | 2.30 | |
| 217986_s_at | NM_013448 | BAZ1A | bromodomain adjacent to zinc finger | −0.49 | 9.1E−03 | 5.42 | 2.67 | 6.94 | 4.27 | |
| | | | domain, 1A | | | | | | | |
| 210093_s_at | AF067173 | MAGOH | mago-nashi homolog, proliferation- | 0.71 | 9.4E−03 | 4.69 | 3.34 | 6.30 | 2.96 | |
| | | | associated (Drosophila) | | | | | | | |
| 212892_at | AW130128 | ZNF282 | zinc finger protein 282 | 0.82 | 9.4E−03 | 2.98 | 1.89 | 4.13 | 2.25 | |
| 240793_at | BF224054 | TTN | Titin | −0.68 | 9.5E−03 | 3.86 | 2.91 | 5.08 | 2.16 | |
| 241812_at | AV648669 | LOC26010 | viral DNA polymerase-transactivated | −0.63 | 9.5E−03 | 1.48 | 0.88 | 5.30 | 4.42 | |
| | | | protein 6 | | | | | | | |
| 233587_s_at | AK022852 | SIPA1L2 | signal-induced proliferation-associated | −0.58 | 9.5E−03 | 5.36 | 3.64 | 7.70 | 4.06 | |
| | | | 1 like 2 | | | | | | | |
| 213988_s_at | BE971383 | SAT1 | spermidine/spermine N1-acetyltransferase 1 | −0.79 | 9.6E−03 | 7.68 | 6.40 | 9.33 | 2.93 | |
| 241599_at | AW014922 | LSM11 | LSM11, U7 small nuclear RNA associated | 0.88 | 9.8E−03 | 2.89 | 1.81 | 4.14 | 2.33 | |
| 241368_at | AI190693 | LSDP5 | lipid storage droplet protein 5 | −0.69 | 9.9E−03 | 4.32 | 2.86 | 5.77 | 2.91 | |
| 200032_s_at | NM_000661 | RPL9 | ribosomal protein L9 | | | | | | | Y |
| 202948_at | NM_000877 | IL1R1 | interleukin 1 receptor, type I | | | | | | | Y |
| 212512_s_at | AA551784 | CARM1 | coactivator-associated arginine | | | | | | | Y |
| | | | methyltransferase 1 | | | | | | | |
| 225453_x_at | BE733510 | CCDC124 | Full length insert cDNA clone ZD51E04 | | | | | | | Y |
| 230393_at | BF448201 | CUL5 | cullin 5 | | | | | | | Y |
| 238364_x_at | BG231548 | GLI4 | GLI-Kruppel family member GLI4 | | | | | | | Y |
| | | | (GLI4), mRNA | | | | | | | |
| 239866_at | AA705933 | — | — | | | | | | | Y |

TABLE 3

| | | | | co- | | exp. | | | |
|---|---|---|---|---|---|---|---|---|---|
| | | | | effi- | raw. | me- | exp. | exp. | exp. |
| probeset | Accession | gene. symb | gene. title | cient | p. value | dian | min | max | diff |
| 239714_at | AA780063 | PIP5K1B | Phosphatidylinositol-4-phosphate 5-kinase type-1 beta | −1.57 | 6.9E−05 | 3.41 | 2.55 | 4.80 | 2.25 |
| 1558938_ [symbol] | BC043574 | — | — | 1.06 | 3.6E−04 | 4.45 | 3.09 | 5.77 | 2.68 |
| 214364_at | W84525 | MTERFD2 | MTERF domain containing 2 | −1.35 | 4.6E−04 | 3.30 | 2.00 | 4.62 | 2.61 |
| 240934_at | AI801975 | PIP5K1B | Phosphatidylinositol-4-phosphate 5-kinase type-1 beta | −1.51 | 5.7E−04 | 3.51 | 2.16 | 4.93 | 2.77 |
| 240980_at | R61819 | — | — | 1.32 | 7.1E−04 | 2.22 | 1.58 | 4.29 | 2.71 |
| 243187_at | AA888821 | PVRL2 | Poliovirus receptor-related protein 2 Precursor | 1.02 | 1.1E−03 | 2.25 | 1.48 | 4.09 | 2.61 |
| 225831_at | AW016830 | LUZP1 | leucine zipper protein 1 | −2.06 | 1.1E−03 | 4.16 | 3.58 | 6.99 | 3.41 |
| 234618_at | AL049434 | PHTF1 | Putative homeodomain transcription factor 1 | 1.02 | 1.2E−03 | 2.54 | 1.79 | 4.40 | 2.61 |
| 232079_s_ [symbol] | BE867789 | PVRL2 | poliovirus receptor-related 2 (herpesvirus entry mediator B) | 0.52 | 1.5E−03 | 3.15 | 2.33 | 6.76 | 4.42 |
| 231886_at | AL137655 | DKFZP434 | similar to hypothetical protein LOC284701 | 0.62 | 2.0E−03 | 4.49 | 2.87 | 6.21 | 3.33 |
| 1562743_ [symbol] | BC042873 | ZNF33B | Zinc finger protein 33B (ZNF33B), mRNA | −1.14 | 2.0E−03 | 3.62 | 2.32 | 4.74 | 2.42 |
| 242109_at | AI038577 | SYTL3 | CDNA FLJ61334 complete cds, moderately similar to | 1.10 | 2.2E−03 | 2.83 | 1.90 | 4.43 | 2.53 |
| | | | Synaptotagmin-like protein 3 | | | | | | |
| 239274_at | AV729557 | PICALM | Phosphatidylinositol-binding clathrin assembly protein | 1.08 | 2.4E−03 | 6.10 | 5.00 | 7.13 | 2.13 |
| 212811_x_ [symbol] | AI889380 | SLC1A4 | solute carrier family 1 (glutamate/neutral | 0.99 | 2.4E−03 | 3.91 | 2.59 | 4.86 | 2.27 |
| | | | amino acid transporter), member 4 | | | | | | |
| 229643_at | AI857933 | ITGA6 | Integrin alpha 6B [human, mRNA Partial, 528 nt] | −1.02 | 2.5E−03 | 3.75 | 2.86 | 5.20 | 2.34 |
| 235971_at | AI147211 | — | — | 0.88 | 2.5E−03 | 3.59 | 2.63 | 5.66 | 3.04 |
| 210113_s_ [symbol] | AF310105 | NLRP1 | NLR family, pyrin domain containing 1 | −1.18 | 2.8E−03 | 5.50 | 4.01 | 6.55 | 2.54 |
| 216145_at | AL137713 | — | — | −1.28 | 2.9E−03 | 2.81 | 2.20 | 4.25 | 2.05 |
| 206494_s_ [symbol] | NM_000419 | ITGA2B | integrin, alpha 2b (platelet glycoprotein IIb of | −0.89 | 3.0E−03 | 3.97 | 2.64 | 5.76 | 3.12 |
| | | | IIb/IIIa complex, antigen CD41) | | | | | | |
| 240671_at | H38635 | GYPC | Glycophorin-C | −0.92 | 3.0E−03 | 3.54 | 2.49 | 5.20 | 2.71 |
| 231721_at | AF356518 | JAM3 | junctional adhesion molecule 3 | −0.58 | 3.2E−03 | 4.10 | 2.43 | 6.17 | 3.75 |
| 217876_at | NM_012087 | GTF3C5 | general transcription factor IIIC, polypeptide 5, 63 kDa | −1.13 | 3.3E−03 | 4.24 | 3.18 | 5.23 | 2.05 |
| 217179_x_ [symbol] | X79782 | — | — | 0.87 | 3.3E−03 | 4.56 | 3.83 | 6.66 | 2.83 |
| 225685_at | AI801777 | — | — | 0.97 | 3.7E−03 | 6.32 | 5.29 | 7.46 | 2.17 |
| 219348_at | NM_018467 | USE1 | unconventional SNARE in the ER 1 homolog | −1.30 | 3.7E−03 | 6.10 | 5.30 | 8.00 | 2.70 |
| | | | (S. cerevisiae) | | | | | | |
| 243106_at | AA916861 | CLEC12A | C-type lectin protein CLL-1 | 0.30 | 3.7E−03 | 3.93 | 1.90 | 7.11 | 5.22 |
| 209589_s_ [symbol] | AF025304 | EPHB2 | EPH receptor B2 | −0.79 | 3.8E−03 | 3.37 | 2.11 | 5.25 | 3.15 |
| 209006_s_ [symbol] | AF247168 | C1orf63 | chromosome 1 open reading frame 63 | 1.03 | 4.0E−03 | 5.89 | 4.66 | 7.63 | 2.97 |
| 238080_at | BF195052 | B4GALNT [symbol] | beta-1,4-N-acetyl-galactosaminyl transferase 4 | −1.04 | 4.3E−03 | 3.13 | 2.28 | 4.47 | 2.19 |
| 1564443_ [symbol] | AF529010 | DLEU2 | deleted in lymphocytic leukemia 2 (non-protein coding) | 0.75 | 4.3E−03 | 4.55 | 3.00 | 6.47 | 3.47 |
| 1568706_ [symbol] | AF318328 | AVIL | Advillin, mRNA (cDNA clone MGC: 133244 | 0.88 | 4.5E−03 | 5.08 | 3.85 | 6.02 | 2.16 |
| | | | IMAGE: 40035028) | | | | | | |
| 238987_at | AL574435 | B4GALT1 | Clone p4betaGT/3 beta-1,4-galactosyltransferase | 0.93 | 4.5E−03 | 3.24 | 1.82 | 4.52 | 2.70 |
| 216956_s_ [symbol] | AF098114 | ITGA2B | integrin, alpha 2b (platelet glycoprotein IIb of | −0.60 | 4.5E−03 | 4.45 | 2.94 | 6.40 | 3.46 |
| | | | IIb/IIIa complex, antigen CD41) | | | | | | |
| 231057_at | AU144266 | MTMR2 | Myotubularin-related protein 2 | 1.14 | 4.7E−03 | 2.91 | 2.07 | 4.22 | 2.15 |
| 234948_at | AK026640 | SLC27A5 | solute carrier family 27 (fatty acid transporter), member 5 | −1.12 | 4.7E−03 | 3.73 | 2.92 | 4.97 | 2.05 |
| 228040_at | AW294192 | MGC2188 [symbol] | hypothetical locus MGC21881 | 0.89 | 4.8E−03 | 3.31 | 2.13 | 5.03 | 2.90 |
| 202874_s_ [symbol] | NM_001695 | ATP6V1C1 | ATPase, H+ transporting, lysosomal 42 kDa, V1 | 0.81 | 5.0E−03 | 5.48 | 4.00 | 6.98 | 2.98 |
| | | | subunit C1 | | | | | | |
| 234047_at | AK024127 | — | — | 1.06 | 5.6E−03 | 3.83 | 2.96 | 4.98 | 2.02 |
| 231174_s_ [symbol] | H92979 | — | — | 1.06 | 5.8E−03 | 2.00 | 1.11 | 4.39 | 3.28 |
| 212592_at | AV733266 | IGJ | immunoglobulin J polypeptide, linker protein for | 0.31 | 6.0E−03 | 3.38 | 1.38 | 8.41 | 7.03 |
| | | | immunoglobulin alpha and mu polypeptides | | | | | | |
| 218618_s_ [symbol] | NM_022763 | FNDC3B | fibronectin type III domain containing 3B | 0.75 | 6.2E−03 | 5.19 | 3.78 | 6.54 | 2.76 |
| 1552703_ [symbol] | NM_052889 | CARD16 | caspase recruitment domain family, member 16 | 0.97 | 6.2E−03 | 8.38 | 7.16 | 9.60 | 2.44 |
| 236458_at | BE875072 | — | — | −1.06 | 6.2E−03 | 2.07 | 1.33 | 4.02 | 2.69 |
| 202948_at | NM_000877 | IL1R1 | interleukin 1 receptor, type I | 0.43 | 6.3E−03 | 3.44 | 1.49 | 5.81 | 4.33 |
| 1562137_ [symbol] | AF147388 | ADAM10 | Disintegrin and metalloproteinase domain-containing | 0.96 | 6.4E−03 | 3.57 | 2.05 | 4.72 | 2.67 |
| | | | protein 10 Precursor | | | | | | |
| 1552398_ [symbol] | NM_138337 | CLEC12A | C-type lectin domain family 12, member A | 0.31 | 6.4E−03 | 5.84 | 4.09 | 8.70 | 4.61 |
| 222692_s_ [symbol] | BF444916 | FNDC3B | fibronectin type III domain containing 3B | 0.90 | 6.6E−03 | 5.33 | 4.36 | 6.62 | 2.26 |
| 203129_s_ [symbol] | BF059313 | KIF5C | kinesin family member 5C | −0.92 | 7.2E−03 | 4.77 | 3.49 | 5.99 | 2.50 |
| 1555281_ [symbol] | BC007934 | ARMC8 | armadillo repeat containing 8 | −0.88 | 7.4E−03 | 5.82 | 4.00 | 6.87 | 2.88 |
| 229180_at | AI685931 | WWC1 | KIBRA protein (KIBRA) | −1.34 | 7.5E−03 | 3.00 | 2.24 | 4.54 | 2.31 |
| 207500_at | NM_004347 | CASP5 | caspase 5, apoptosis-related cysteine peptidase | 0.70 | 7.5E−03 | 3.89 | 2.30 | 6.14 | 3.85 |
| 232963_at | BF725688 | RFWD2 | ring finger and WD repeat domain 2 | 0.82 | 7.5E−03 | 4.55 | 3.54 | 5.93 | 2.39 |
| 233504_at | AA629020 | C9orf84 | chromosome 9 open reading frame 84 | 0.70 | 7.6E−03 | 5.06 | 3.39 | 6.48 | 3.09 |
| 222693_at | BF444916 | FNDC3B | fibronectin type III domain containing 3B | 0.60 | 7.6E−03 | 3.94 | 2.88 | 5.73 | 2.85 |
| 222411_s_ [symbol] | AW087870 | SSR3 | signal sequence receptor, gamma (translocon-associated | 0.86 | 8.0E−03 | 5.55 | 4.42 | 6.82 | 2.40 |
| | | | protein gamma) | | | | | | |
| 211368_s_ [symbol] | U13700 | CASP1 | caspase 1, apoptosis-related cysteine peptidase (interleukin | 0.79 | 8.0E−03 | 6.41 | 4.86 | 7.73 | 2.86 |
| | | | 1, beta, convertase) | | | | | | |
| 232472_at | AK022461 | FNDC3B | Fibronectin type III domain-containing protein 3B | 0.63 | 8.1E−03 | 3.86 | 2.60 | 5.60 | 3.00 |
| 218435_at | NM_013238 | DNAJC15 | DnaJ (Hsp40) homolog, subfamily C, member 15 | 0.64 | 8.3E−03 | 5.15 | 3.55 | 6.54 | 2.99 |
| 215093_at | U82671 | NSDHL | NAD(P) dependent steroid dehydrogenase-like | 0.54 | 8.3E−03 | 3.50 | 1.82 | 6.28 | 4.46 |
| 209091_s_ [symbol] | AF263293 | SH3GLB1 | SH3-domain GRB2-like endophilin B1 | 1.02 | 8.3E−03 | 7.39 | 6.50 | 8.58 | 2.08 |
| 238589_s_ [symbol] | AW601184 | ATXN2 | Ataxin-2 | 0.71 | 8.3E−03 | 4.82 | 3.32 | 6.35 | 3.03 |
| 1558011_ [symbol] | BM823647 | — | — | 0.64 | 8.4E−03 | 6.95 | 5.39 | 8.67 | 3.28 |
| 205877_s_ [symbol] | NM_017590 | ZC3H7B | zinc finger CCCH-type containing 7B | −1.06 | 8.4E−03 | 4.80 | 3.36 | 5.74 | 2.38 |
| 239603_x_ [symbol] | AI082479 | FBXO11 | F-box only protein 11 | 0.91 | 8.6E−03 | 5.78 | 4.81 | 8.59 | 3.78 |
| 214594_x_ [symbol] | BG252666 | ATP8B1 | ATPase, class I, type 8B, member 1 | 0.81 | 8.6E−03 | 6.40 | 4.97 | 7.27 | 2.30 |
| 206267_s_ [symbol] | NM_002378 | MATK | megakaryocyte-associated tyrosine kinase | −0.87 | 8.8E−03 | 3.65 | 2.62 | 4.63 | 2.01 |
| 209970_x_ [symbol] | M87507 | CASP1 | caspase 1, apoptosis-related cysteine peptidase (interleukin | 0.80 | 8.9E−03 | 6.58 | 5.06 | 7.70 | 2.64 |
| | | | 1, beta, convertase) | | | | | | |
| 232078_at | BE867789 | PVRL2 | poliovirus receptor-related 2 (herpesvirus entry mediator B) | 0.40 | 9.0E−03 | 2.53 | 1.29 | 6.24 | 4.95 |
| 228718_at | AI379070 | ZNF44 | zinc finger protein 44 | −0.94 | 9.0E−03 | 3.32 | 2.63 | 5.23 | 2.60 |
| 232417_x_ [symbol] | AU150300 | ZDHHC11 | zinc finger, DHHC-type containing 11 | −1.17 | 9.1E−03 | 4.70 | 3.97 | 5.99 | 2.02 |
| 224917_at | BF674052 | MIR21 | microRNA 21 | 0.74 | 9.1E−03 | 6.66 | 5.36 | 8.32 | 2.96 |
| 239124_at | AA002064 | PITPNA | Phosphatidylinositol transfer protein alpha isoform | 0.83 | 9.1E−03 | 3.23 | 1.98 | 4.50 | 2.52 |
| 219476_at | NM_024115 | C1orf116 | chromosome 1 open reading frame 116 | −0.91 | 9.2E−03 | 2.39 | 1.70 | 4.31 | 2.61 |
| 218415_at | NM_018668 | VPS33B | vacuolar protein sorting 33 homolog B (yeast) | −0.67 | 9.4E−03 | 4.31 | 2.31 | 5.85 | 3.55 |
| 219700_at | NM_020405 | PLXDC1 | plexin domain containing 1 | −0.65 | 9.4E−03 | 4.36 | 2.52 | 5.65 | 3.13 |
| 243980_at | AW978739 | ZNF594 | MRNA; cDNA DKFZp667J055 (from clone | −0.96 | 9.4E−03 | 3.31 | 1.93 | 4.40 | 2.47 |
| | | | DKFZp667J055) | | | | | | |
| 1554482_ [symbol] | BC002847 | SAR1B | SAR1 homolog B (S. cerevisiae) | 0.66 | 9.5E−03 | 4.05 | 2.56 | 5.37 | 2.81 |
| 215191_at | AW836210 | FBXL11 | Lysine-specific demethylase 2A | 0.56 | 9.7E−03 | 3.39 | 1.96 | 5.14 | 3.18 |
| 211366_x_ [symbol] | U13698 | CASP1 | caspase 1, apoptosis-related cysteine peptidase (interleukin | 0.93 | 9.9E−03 | 7.42 | 6.32 | 8.70 | 2.37 |
| | | | 1, beta, convertase) | | | | | | |
| 244308_at | BF514096 | SYT15 | Chr10 synaptotagmin (CHR10SYT gene) | 0.73 | 9.9E−03 | 2.48 | 1.57 | 5.12 | 3.55 |
| 210219_at | U36501 | SP100 | SP100 nuclear antigen | 0.64 | 1.0E−02 | 1.67 | 1.10 | 6.37 | 5.28 |

[symbol]

indicates data missing or illegible when filed

Table 5a

| N1:54630 | ID | ACR111Dn3 | ACR62Dn3 | EULAR111Dn3 | EULAR45Dn3 | DAS28wk16_110Dn3 |
|---|---|---|---|---|---|---|
| 91 | 200053_at | 0 | 0 | 45 | 0 | 0 |
| 145 | 200600_at | 0 | 0 | 94 | 84 | 0 |
| 210 | 200665_s_at | 0 | 0 | 41 | 0 | 0 |
| 495 | 200950_at | 0 | 81 | 0 | 0 | 0 |
| 523 | 200978_at | 0 | 56 | 0 | 0 | 0 |
| 617 | 201072_s_at | 0 | 83 | 0 | 0 | 0 |
| 670 | 201125_s_at | 0 | 0 | 19 | 33 | 0 |
| 708 | 201163_s_at | 0 | 0 | 0 | 0 | 0 |
| 771 | 201226_at | 0 | 33 | 0 | 0 | 0 |
| 846 | 201301_s_at | 0 | 0 | 0 | 0 | 85 |
| 915 | 201370_s_at | 0 | 0 | 0 | 0 | 0 |
| 931 | 201386_s_at | 0 | 0 | 0 | 0 | 0 |
| 962 | 201417_at | 0 | 0 | 0 | 0 | 0 |
| 1005 | 201460_at | 26 | 0 | 0 | 0 | 0 |
| 1007 | 201462_at | 56 | 0 | 0 | 0 | 0 |
| 1013 | 201468_s_at | 0 | 0 | 0 | 86 | 0 |
| 1056 | 201511_at | 0 | 0 | 0 | 0 | 0 |
| 1081 | 201536_at | 0 | 0 | 0 | 0 | 0 |
| 1226 | 201681_s_at | 0 | 0 | 93 | 97 | 0 |
| 1429 | 201884_at | 0 | 0 | 0 | 73 | 0 |
| 1450 | 201905_s_at | 0 | 0 | 0 | 0 | 0 |
| 1456 | 201911_s_at | 0 | 0 | 0 | 0 | 0 |
| 1519 | 201974_s_at | 0 | 71 | 0 | 0 | 0 |
| 1528 | 201983_s_at | 0 | 0 | 0 | 0 | 0 |
| 1581 | 202036_s_at | 0 | 53 | 0 | 0 | 0 |
| 1686 | 202141_s_at | 0 | 0 | 0 | 0 | 0 |
| 1869 | 202324_s_at | 0 | 0 | 0 | 0 | 0 |
| 1910 | 202365_at | 48 | 0 | 0 | 0 | 0 |
| 2062 | 202517_at | 97 | 0 | 0 | 0 | 0 |
| 2124 | 202579_x_at | 0 | 0 | 28 | 0 | 0 |
| 2235 | 202690_s_at | 0 | 0 | 0 | 0 | 0 |
| 2327 | 202782_s_at | 0 | 0 | 91 | 0 | 0 |
| 2411 | 202866_at | 0 | 0 | 27 | 26 | 0 |
| 2614 | 203068_at | 9 | 0 | 0 | 0 | 0 |
| 2717 | 203172_at | 0 | 0 | 0 | 0 | 0 |
| 2767 | 203222_s_at | 0 | 0 | 0 | 0 | 0 |
| 2815 | 203271_s_at | 95 | 0 | 0 | 0 | 0 |
| 2823 | 203279_at | 0 | 27 | 0 | 0 | 0 |
| 2837 | 203293_s_at | 0 | 0 | 16 | 99 | 0 |
| 2997 | 203453_at | 0 | 0 | 0 | 0 | 0 |
| 3131 | 203587_at | 0 | 0 | 0 | 0 | 58 |
| 3435 | 203891_s_at | 0 | 0 | 49 | 94 | 0 |
| 3446 | 203902_at | 0 | 0 | 0 | 0 | 0 |
| 3455 | 203911_at | 0 | 0 | 0 | 0 | 0 |
| 3511 | 203967_at | 0 | 0 | 0 | 0 | 0 |
| 3585 | 204041_at | 0 | 0 | 87 | 31 | 0 |
| 3661 | 204117_at | 0 | 67 | 0 | 0 | 0 |
| 3937 | 204393_s_at | 0 | 68 | 0 | 0 | 0 |
| 3942 | 204398_s_at | 0 | 0 | 26 | 0 | 0 |
| 3966 | 204422_s_at | 0 | 0 | 0 | 0 | 76 |
| 3982 | 204438_at | 0 | 63 | 0 | 0 | 0 |
| 4000 | 204456_s_at | 0 | 0 | 0 | 0 | 0 |
| 4171 | 204627_s_at | 0 | 0 | 39 | 65 | 0 |
| 4316 | 204772_s_at | 0 | 0 | 0 | 0 | 6 |
| 4346 | 204802_at | 0 | 0 | 0 | 0 | 0 |
| 4493 | 204949_at | 0 | 0 | 17 | 0 | 0 |
| 4497 | 204953_at | 77 | 61 | 0 | 0 | 0 |
| 4506 | 204962_s_at | 0 | 0 | 24 | 77 | 0 |
| 4528 | 204984_at | 0 | 15 | 0 | 0 | 0 |
| 4805 | 205261_at | 0 | 32 | 0 | 0 | 0 |
| 4825 | 205281_s_at | 0 | 0 | 0 | 0 | 79 |
| 4838 | 205294_at | 0 | 0 | 0 | 0 | 0 |
| 4976 | 205432_at | 0 | 0 | 0 | 0 | 0 |
| 5175 | 205631_at | 0 | 0 | 0 | 8 | 0 |
| 5405 | 205861_at | 0 | 0 | 0 | 0 | 0 |
| 5414 | 205870_at | 0 | 0 | 0 | 60 | 0 |
| 5536 | 205992_s_at | 0 | 0 | 0 | 0 | 0 |
| 5622 | 206079_at | 64 | 0 | 0 | 0 | 0 |
| 5689 | 206146_s_at | 0 | 0 | 82 | 0 | 0 |
| 5868 | 206325_at | 0 | 0 | 0 | 62 | 0 |
| 5970 | 206427_s_at | 0 | 0 | 0 | 0 | 0 |
| 6036 | 206493_at | 0 | 0 | 14 | 38 | 0 |
| 6046 | 206503_x_at | 99 | 0 | 0 | 0 | 0 |
| 6104 | 206561_s_at | 0 | 0 | 0 | 0 | 0 |
| 6116 | 206573_at | 98 | 0 | 0 | 0 | 0 |
| 6152 | 206609_at | 0 | 0 | 0 | 0 | 5 |
| 6177 | 206634_at | 0 | 48 | 0 | 0 | 95 |
| 6178 | 206635_at | 0 | 0 | 0 | 0 | 0 |
| 6254 | 206711_at | 0 | 0 | 0 | 0 | 57 |
| 6273 | 206730_at | 32 | 55 | 0 | 0 | 18 |
| 6341 | 206798_x_at | 0 | 0 | 0 | 0 | 0 |
| 6495 | 206952_at | 0 | 0 | 0 | 4 | 0 |
| 6637 | 207094_at | 80 | 0 | 0 | 0 | 0 |
| 6639 | 207096_at | 0 | 0 | 0 | 0 | 83 |
| 6777 | 207235_s_at | 0 | 0 | 0 | 0 | 4 |
| 6869 | 207328_at | 0 | 72 | 0 | 0 | 0 |
| 6905 | 207365_x_at | 83 | 0 | 0 | 0 | 0 |
| 6968 | 207429_at | 0 | 46 | 0 | 0 | 0 |
| 7108 | 207570_at | 0 | 0 | 32 | 74 | 0 |
| 7136 | 207598_x_at | 60 | 0 | 0 | 0 | 0 |
| 7201 | 207663_x_at | 0 | 0 | 0 | 0 | 0 |
| 7231 | 207693_at | 0 | 0 | 0 | 78 | 0 |
| 7268 | 207730_x_at | 53 | 0 | 0 | 0 | 0 |
| 7353 | 207817_at | 0 | 0 | 0 | 0 | 0 |
| 7400 | 207864_at | 46 | 0 | 0 | 0 | 0 |
| 7545 | 208019_at | 0 | 0 | 0 | 0 | 82 |
| 7634 | 208108_s_at | 0 | 0 | 0 | 0 | 0 |
| 7709 | 208186_s_at | 0 | 0 | 0 | 0 | 0 |
| 7731 | 208209_s_at | 0 | 0 | 0 | 23 | 0 |
| 7749 | 208227_x_at | 86 | 0 | 0 | 0 | 0 |
| 7812 | 208291_s_at | 0 | 0 | 0 | 0 | 0 |
| 7901 | 208383_s_at | 0 | 0 | 0 | 0 | 0 |
| 7991 | 208476_s_at | 0 | 0 | 0 | 0 | 0 |
| 8286 | 208774_at | 71 | 0 | 0 | 0 | 0 |
| 8401 | 208889_s_at | 0 | 0 | 0 | 0 | 0 |
| 8631 | 209120_at | 0 | 0 | 79 | 0 | 0 |
| 8799 | 209288_s_at | 0 | 0 | 0 | 0 | 0 |
| 8874 | 209364_at | 0 | 0 | 0 | 0 | 88 |
| 9068 | 209559_at | 0 | 0 | 0 | 0 | 0 |
| 9157 | 209651_at | 0 | 0 | 0 | 0 | 0 |
| 9240 | 209735_at | 0 | 0 | 0 | 80 | 0 |
| 9296 | 209791_at | 0 | 0 | 2 | 0 | 0 |
| 9337 | 209834_at | 0 | 0 | 0 | 0 | 55 |
| 9362 | 209859_at | 0 | 0 | 0 | 92 | 0 |
| 9525 | 210023_s_at | 66 | 0 | 0 | 0 | 0 |
| 9541 | 210039_s_at | 0 | 22 | 0 | 0 | 0 |
| 9596 | 210095_s_at | 0 | 0 | 0 | 0 | 0 |
| 9633 | 210132_at | 0 | 0 | 0 | 0 | 0 |
| 9696 | 210195_s_at | 0 | 28 | 0 | 0 | 0 |
| 9728 | 210230_at | 85 | 0 | 0 | 0 | 0 |
| 9742 | 210245_at | 0 | 75 | 0 | 0 | 0 |
| 9766 | 210269_s_at | 0 | 0 | 0 | 0 | 0 |
| 9826 | 210330_at | 0 | 0 | 0 | 0 | 71 |
| 9915 | 210420_at | 40 | 8 | 0 | 0 | 52 |
| 10007 | 210520_at | 0 | 0 | 0 | 59 | 0 |
| 10093 | 210611_s_at | 16 | 39 | 0 | 0 | 0 |
| 10143 | 210661_at | 0 | 0 | 0 | 0 | 0 |
| 10447 | 210983_s_at | 0 | 0 | 1 | 1 | 0 |
| 10489 | 211026_s_at | 0 | 0 | 0 | 0 | 0 |
| 10757 | 211320_s_at | 0 | 0 | 0 | 0 | 0 |
| 10773 | 211338_at | 0 | 0 | 0 | 0 | 41 |
| 10797 | 211364_at | 0 | 0 | 0 | 0 | 70 |
| 10884 | 211458_s_at | 0 | 0 | 0 | 63 | 0 |
| 10893 | 211467_s_at | 0 | 5 | 0 | 0 | 0 |
| 10895 | 211469_s_at | 0 | 43 | 0 | 0 | 0 |
| 10992 | 211570_s_at | 0 | 0 | 0 | 0 | 0 |
| 11058 | 211637_x_at | 0 | 0 | 97 | 0 | 0 |
| 11061 | 211640_x_at | 0 | 0 | 51 | 39 | 0 |
| 11240 | 211827_s_at | 0 | 0 | 0 | 0 | 3 |
| 11328 | 211923_s_at | 57 | 10 | 0 | 0 | 2 |
| 11495 | 212092_at | 0 | 0 | 0 | 82 | 0 |
| 11500 | 212097_at | 0 | 34 | 0 | 0 | 0 |
| 11645 | 212242_at | 0 | 0 | 37 | 0 | 0 |
| 11867 | 212465_at | 0 | 0 | 0 | 46 | 0 |
| 11918 | 212516_at | 0 | 0 | 38 | 0 | 0 |
| 11930 | 212528_at | 0 | 0 | 0 | 0 | 73 |
| 12160 | 212758_s_at | 20 | 0 | 0 | 44 | 31 |
| 12199 | 212797_at | 0 | 58 | 0 | 0 | 0 |
| 12294 | 212892_at | 0 | 0 | 0 | 0 | 0 |
| 12391 | 212991_at | 0 | 90 | 0 | 0 | 0 |
| 12680 | 213282_at | 0 | 0 | 0 | 0 | 0 |
| 12698 | 213300_at | 0 | 0 | 83 | 0 | 0 |
| 12721 | 213323_s_at | 0 | 0 | 0 | 0 | 17 |
| 12750 | 213352_at | 62 | 0 | 0 | 0 | 0 |
| 12820 | 213422_s_at | 0 | 0 | 6 | 21 | 0 |
| 13420 | 214024_s_at | 0 | 74 | 0 | 0 | 0 |
| 13433 | 214037_s_at | 0 | 0 | 25 | 52 | 0 |
| 13576 | 214180_at | 0 | 0 | 0 | 13 | 0 |
| 13717 | 214321_at | 47 | 0 | 0 | 0 | 36 |
| 13806 | 214410_at | 0 | 0 | 0 | 0 | 0 |
| 13834 | 214438_at | 0 | 0 | 48 | 0 | 0 |
| 13970 | 214575_s_at | 3 | 0 | 0 | 0 | 0 |
| 14067 | 214674_at | 0 | 0 | 0 | 0 | 97 |
| 14135 | 214742_at | 0 | 0 | 0 | 0 | 0 |
| 14159 | 214766_s_at | 92 | 0 | 0 | 0 | 37 |
| 14162 | 214769_at | 0 | 0 | 54 | 72 | 0 |
| 14170 | 214777_at | 0 | 0 | 61 | 0 | 0 |
| 14213 | 214821_at | 0 | 0 | 0 | 0 | 0 |
| 14276 | 214884_at | 0 | 0 | 0 | 0 | 0 |
| 14290 | 214898_x_at | 0 | 0 | 0 | 0 | 27 |
| 14350 | 214958_s_at | 0 | 1 | 0 | 0 | 65 |
| 14398 | 215006_at | 94 | 0 | 0 | 0 | 0 |
| 14407 | 215016_x_at | 0 | 14 | 0 | 0 | 0 |
| 14446 | 215055_at | 0 | 0 | 0 | 0 | 0 |
| 14581 | 215190_at | 0 | 0 | 0 | 0 | 0 |
| 14642 | 215251_at | 0 | 0 | 0 | 0 | 0 |
| 14913 | 215523_at | 73 | 0 | 0 | 0 | 0 |
| 14980 | 215590_x_at | 0 | 0 | 0 | 0 | 44 |
| 14994 | 215604_x_at | 18 | 0 | 0 | 0 | 0 |
| 15045 | 215655_at | 0 | 0 | 0 | 0 | 0 |
| 15099 | 215709_at | 100 | 0 | 0 | 0 | 0 |
| 15122 | 215732_s_at | 0 | 0 | 80 | 66 | 0 |
| 15127 | 215737_x_at | 0 | 0 | 46 | 0 | 0 |
| 15233 | 215843_s_at | 49 | 0 | 0 | 0 | 21 |
| 15309 | 215919_s_at | 0 | 0 | 0 | 0 | 0 |
| 15525 | 216136_at | 0 | 0 | 0 | 0 | 0 |
| 15612 | 216223_at | 0 | 26 | 0 | 0 | 0 |
| 15618 | 216229_x_at | 19 | 0 | 0 | 0 | 0 |
| 15818 | 216430_x_at | 0 | 0 | 52 | 0 | 0 |
| 15871 | 216483_s_at | 0 | 0 | 90 | 19 | 0 |
| 15953 | 216566_at | 0 | 0 | 11 | 29 | 0 |
| 16315 | 216928_at | 0 | 0 | 0 | 0 | 0 |
| 16393 | 217006_x_at | 0 | 0 | 0 | 55 | 0 |
| 16398 | 217011_at | 0 | 89 | 0 | 0 | 11 |
| 16421 | 217034_at | 0 | 0 | 99 | 79 | 0 |
| 16510 | 217126_at | 63 | 42 | 0 | 0 | 0 |
| 16521 | 217137_x_at | 0 | 0 | 22 | 6 | 0 |
| 16547 | 217163_at | 0 | 0 | 0 | 0 | 0 |
| 16560 | 217176_s_at | 28 | 0 | 0 | 0 | 0 |
| 16584 | 217200_x_at | 0 | 0 | 0 | 0 | 0 |
| 16606 | 217222_at | 0 | 0 | 65 | 0 | 0 |
| 16673 | 217291_at | 0 | 0 | 0 | 0 | 0 |
| 16686 | 217304_at | 0 | 0 | 0 | 0 | 0 |
| 16863 | 217481_x_at | 0 | 0 | 85 | 0 | 0 |
| 16865 | 217483_at | 0 | 0 | 0 | 0 | 7 |
| 16922 | 217540_at | 0 | 0 | 0 | 0 | 9 |
| 16961 | 217579_x_at | 0 | 0 | 0 | 96 | 0 |
| 17051 | 217669_s_at | 69 | 0 | 0 | 0 | 0 |
| 17091 | 217709_at | 0 | 0 | 0 | 0 | 0 |
| 17248 | 217866_at | 34 | 0 | 0 | 0 | 0 |
| 17294 | 217912_at | 65 | 78 | 0 | 0 | 0 |
| 17514 | 218132_s_at | 29 | 0 | 0 | 0 | 0 |
| 17526 | 218144_s_at | 0 | 0 | 0 | 0 | 0 |
| 17680 | 218298_s_at | 0 | 0 | 0 | 0 | 0 |
| 17736 | 218354_at | 0 | 0 | 0 | 53 | 0 |
| 17757 | 218375_at | 0 | 0 | 0 | 0 | 0 |
| 17785 | 218403_at | 0 | 0 | 0 | 0 | 0 |
| 17803 | 218421_at | 12 | 0 | 0 | 0 | 0 |
| 17821 | 218439_s_at | 0 | 0 | 0 | 0 | 96 |
| 17943 | 218561_s_at | 0 | 3 | 0 | 0 | 0 |
| 17955 | 218573_at | 0 | 0 | 0 | 0 | 0 |
| 17959 | 218577_at | 0 | 0 | 0 | 0 | 0 |
| 18051 | 218670_at | 0 | 0 | 21 | 49 | 0 |
| 18066 | 218685_s_at | 0 | 0 | 0 | 0 | 0 |
| 18074 | 218693_at | 0 | 0 | 0 | 0 | 35 |
| 18196 | 218815_s_at | 0 | 0 | 0 | 0 | 61 |
| 18263 | 218882_s_at | 0 | 0 | 0 | 0 | 0 |
| 18295 | 218914_at | 0 | 80 | 0 | 0 | 0 |
| 18325 | 218944_at | 0 | 0 | 0 | 0 | 81 |
| 18344 | 218963_s_at | 0 | 0 | 0 | 37 | 0 |
| 18474 | 219093_at | 0 | 0 | 0 | 0 | 0 |
| 18663 | 219282_s_at | 0 | 0 | 0 | 61 | 0 |
| 18692 | 219311_at | 0 | 0 | 0 | 0 | 62 |
| 18790 | 219409_at | 0 | 9 | 0 | 0 | 0 |
| 18870 | 219489_s_at | 0 | 0 | 0 | 0 | 0 |
| 18930 | 219549_s_at | 0 | 0 | 70 | 0 | 0 |
| 18946 | 219565_at | 0 | 0 | 0 | 0 | 0 |
| 19008 | 219627_at | 82 | 0 | 0 | 0 | 0 |
| 19024 | 219643_at | 0 | 0 | 0 | 0 | 0 |
| 19452 | 220071_x_at | 21 | 0 | 0 | 0 | 0 |
| 19474 | 220093_at | 0 | 0 | 73 | 0 | 0 |
| 19680 | 220299_at | 0 | 0 | 0 | 0 | 0 |
| 19733 | 220352_x_at | 23 | 0 | 0 | 0 | 0 |
| 19837 | 220456_at | 0 | 0 | 0 | 54 | 0 |
| 19887 | 220506_at | 0 | 0 | 0 | 0 | 0 |
| 19973 | 220592_at | 0 | 98 | 0 | 0 | 0 |
| 20125 | 220744_s_at | 0 | 0 | 0 | 0 | 0 |
| 20192 | 220811_at | 0 | 0 | 0 | 0 | 0 |
| 20209 | 220828_s_at | 0 | 0 | 0 | 98 | 0 |
| 20214 | 220833_at | 93 | 0 | 0 | 0 | 0 |
| 20220 | 220839_at | 0 | 0 | 0 | 0 | 75 |
| 20258 | 220877_at | 36 | 0 | 0 | 0 | 0 |
| 20390 | 221009_s_at | 0 | 0 | 0 | 0 | 0 |
| 20401 | 221020_s_at | 0 | 31 | 0 | 0 | 0 |
| 20424 | 221043_at | 0 | 0 | 81 | 0 | 0 |
| 20512 | 221132_at | 0 | 0 | 0 | 0 | 0 |
| 20547 | 221167_s_at | 0 | 0 | 0 | 0 | 53 |
| 20549 | 221169_s_at | 0 | 44 | 0 | 0 | 0 |
| 20690 | 221310_at | 0 | 0 | 0 | 0 | 26 |
| 20752 | 221372_s_at | 0 | 0 | 29 | 18 | 0 |
| 20788 | 221408_x_at | 0 | 0 | 0 | 0 | 0 |
| 20816 | 221436_s_at | 0 | 0 | 0 | 0 | 19 |
| 20956 | 221577_x_at | 0 | 0 | 0 | 0 | 43 |
| 21196 | 221819_at | 0 | 0 | 47 | 0 | 0 |
| 21238 | 221861_at | 0 | 0 | 7 | 7 | 0 |
| 21263 | 221886_at | 0 | 0 | 0 | 0 | 0 |
| 21281 | 221904_at | 67 | 0 | 0 | 0 | 0 |
| 21648 | 222272_x_at | 90 | 0 | 0 | 0 | 0 |
| 21652 | 222276_at | 0 | 0 | 0 | 0 | 42 |
| 21833 | 34858_at | 74 | 0 | 0 | 0 | 0 |
| 21843 | 35265_at | 0 | 0 | 3 | 67 | 0 |
| 21925 | 38149_at | 0 | 0 | 40 | 15 | 0 |
| 21974 | 40420_at | 37 | 0 | 0 | 0 | 0 |
| 22102 | 52837_at | 0 | 0 | 0 | 0 | 0 |
| 22287 | 222439_s_at | 0 | 0 | 0 | 51 | 0 |
| 22352 | 222504_s_at | 0 | 0 | 0 | 0 | 0 |
| 22628 | 222780_s_at | 33 | 0 | 0 | 0 | 0 |
| 22742 | 222894_x_at | 43 | 50 | 0 | 0 | 0 |
| 23095 | 223249_at | 91 | 0 | 0 | 0 | 16 |
| 23127 | 223281_s_at | 0 | 0 | 0 | 0 | 89 |
| 23130 | 223284_at | 0 | 0 | 0 | 35 | 0 |
| 23337 | 223491_at | 0 | 0 | 0 | 0 | 0 |
| 23353 | 223507_at | 0 | 0 | 0 | 0 | 0 |
| 23368 | 223523_at | 0 | 0 | 44 | 0 | 0 |
| 23788 | 223944_at | 10 | 0 | 0 | 0 | 0 |
| 23830 | 223987_at | 0 | 0 | 0 | 0 | 0 |
| 23870 | 224027_at | 0 | 0 | 0 | 0 | 0 |
| 23914 | 224071_at | 0 | 0 | 0 | 0 | 0 |
| 23943 | 224100_s_at | 0 | 70 | 0 | 0 | 0 |
| 24025 | 224184_s_at | 0 | 30 | 0 | 0 | 0 |
| 24093 | 224254_x_at | 0 | 0 | 66 | 0 | 0 |
| 24121 | 224284_x_at | 0 | 0 | 36 | 40 | 0 |
| 24187 | 224351_at | 0 | 0 | 0 | 0 | 46 |
| 24310 | 224480_s_at | 0 | 0 | 89 | 0 | 0 |
| 24359 | 224529_s_at | 0 | 12 | 0 | 0 | 0 |
| 24369 | 224539_s_at | 0 | 0 | 0 | 64 | 0 |
| 24463 | 224635_s_at | 0 | 0 | 0 | 0 | 0 |
| 24489 | 224661_at | 0 | 0 | 0 | 17 | 0 |
| 24610 | 224783_at | 22 | 0 | 0 | 0 | 0 |
| 24619 | 224792_at | 24 | 0 | 0 | 0 | 0 |
| 24752 | 224925_at | 0 | 0 | 0 | 0 | 78 |
| 24753 | 224926_at | 35 | 0 | 0 | 0 | 0 |
| 24828 | 225001_at | 0 | 0 | 9 | 69 | 0 |
| 24903 | 225077_at | 0 | 0 | 0 | 0 | 0 |
| 25062 | 225236_at | 0 | 11 | 0 | 0 | 0 |
| 25121 | 225295_at | 0 | 0 | 0 | 0 | 0 |
| 25156 | 225330_at | 0 | 0 | 0 | 0 | 67 |
| 25197 | 225371_at | 0 | 0 | 0 | 14 | 0 |
| 25312 | 225486_at | 6 | 0 | 0 | 0 | 0 |
| 25404 | 225578_at | 0 | 0 | 0 | 0 | 0 |
| 25461 | 225635_s_at | 0 | 0 | 0 | 0 | 0 |
| 25669 | 225843_at | 0 | 0 | 78 | 0 | 0 |
| 25815 | 225990_at | 0 | 0 | 0 | 88 | 0 |
| 25816 | 225991_at | 0 | 0 | 35 | 0 | 0 |
| 25854 | 226029_at | 54 | 0 | 0 | 0 | 0 |
| 25872 | 226047_at | 51 | 0 | 0 | 0 | 0 |
| 25879 | 226054_at | 0 | 0 | 4 | 16 | 0 |
| 25890 | 226065_at | 0 | 0 | 0 | 0 | 0 |
| 26055 | 226230_at | 76 | 0 | 0 | 0 | 0 |
| 26321 | 226496_at | 0 | 0 | 0 | 0 | 0 |
| 26389 | 226564_at | 0 | 0 | 0 | 0 | 0 |
| 26474 | 226650_at | 78 | 0 | 0 | 0 | 0 |
| 26559 | 226735_at | 0 | 93 | 0 | 0 | 0 |
| 26729 | 226905_at | 0 | 0 | 12 | 10 | 0 |
| 26750 | 226926_at | 0 | 0 | 58 | 100 | 0 |
| 26955 | 227131_at | 61 | 0 | 0 | 0 | 0 |
| 27236 | 227412_at | 0 | 0 | 0 | 0 | 48 |
| 27331 | 227508_at | 96 | 60 | 0 | 0 | 0 |
| 27487 | 227664_at | 0 | 0 | 0 | 0 | 14 |
| 27873 | 228050_at | 0 | 0 | 0 | 0 | 0 |
| 27888 | 228065_at | 31 | 0 | 0 | 0 | 0 |
| 27915 | 228092_at | 0 | 0 | 0 | 0 | 38 |
| 27997 | 228174_at | 0 | 0 | 0 | 0 | 20 |
| 28042 | 228219_s_at | 0 | 21 | 0 | 0 | 0 |
| 28091 | 228268_at | 0 | 0 | 0 | 0 | 0 |
| 28163 | 228340_at | 79 | 0 | 0 | 0 | 0 |
| 28281 | 228458_at | 0 | 96 | 0 | 0 | 0 |
| 28316 | 228493_at | 0 | 0 | 0 | 0 | 0 |
| 28462 | 228639_at | 0 | 49 | 0 | 0 | 0 |
| 28542 | 228719_at | 0 | 88 | 0 | 0 | 0 |
| 28601 | 228778_at | 0 | 0 | 86 | 0 | 0 |
| 28700 | 228877_at | 0 | 0 | 0 | 0 | 0 |
| 28739 | 228916_at | 0 | 0 | 0 | 0 | 0 |
| 28795 | 228972_at | 0 | 0 | 0 | 0 | 0 |
| 28954 | 229131_at | 0 | 0 | 0 | 0 | 0 |
| 29105 | 229282_at | 0 | 0 | 0 | 0 | 0 |
| 29117 | 229294_at | 0 | 0 | 0 | 0 | 0 |
| 29214 | 229391_s_at | 0 | 0 | 0 | 0 | 0 |
| 29502 | 229679_at | 0 | 0 | 0 | 0 | 0 |
| 29858 | 230035_at | 38 | 0 | 0 | 0 | 0 |
| 29949 | 230126_s_at | 39 | 0 | 0 | 0 | 0 |
| 29992 | 230169_at | 8 | 0 | 0 | 0 | 0 |
| 30150 | 230327_at | 0 | 0 | 0 | 0 | 0 |
| 30320 | 230497_at | 0 | 0 | 0 | 0 | 64 |
| 30345 | 230522_s_at | 0 | 0 | 0 | 0 | 0 |
| 30397 | 230574_at | 0 | 0 | 0 | 20 | 0 |
| 30428 | 230605_at | 0 | 0 | 0 | 0 | 0 |
| 30513 | 230690_at | 0 | 0 | 42 | 42 | 0 |
| 30517 | 230694_at | 0 | 0 | 0 | 0 | 0 |
| 30543 | 230720_at | 0 | 0 | 0 | 0 | 0 |
| 30559 | 230736_at | 0 | 0 | 0 | 0 | 60 |
| 30682 | 230859_at | 0 | 84 | 0 | 0 | 0 |
| 30758 | 230935_at | 0 | 0 | 0 | 0 | 0 |
| 30874 | 231051_at | 0 | 87 | 0 | 0 | 0 |
| 30903 | 231080_at | 0 | 0 | 0 | 0 | 0 |
| 31011 | 231188_at | 0 | 0 | 69 | 24 | 0 |
| 31220 | 231397_at | 0 | 59 | 0 | 0 | 1 |
| 31296 | 231473_at | 0 | 20 | 0 | 30 | 32 |
| 31325 | 231502_at | 0 | 0 | 0 | 0 | 0 |
| 31501 | 231678_s_at | 0 | 0 | 0 | 0 | 0 |
| 31544 | 231721_at | 0 | 0 | 0 | 0 | 0 |
| 31826 | 232003_at | 0 | 0 | 95 | 0 | 0 |
| 31897 | 232074_at | 0 | 0 | 92 | 0 | 0 |
| 31902 | 232079_s_at | 0 | 0 | 0 | 32 | 0 |
| 31946 | 232123_at | 0 | 0 | 0 | 0 | 23 |
| 32012 | 232189_at | 0 | 0 | 0 | 0 | 0 |
| 32031 | 232208_at | 0 | 0 | 0 | 0 | 0 |
| 32067 | 232244_at | 75 | 0 | 0 | 0 | 0 |
| 32082 | 232259_s_at | 0 | 0 | 0 | 0 | 0 |
| 32152 | 232329_at | 0 | 0 | 0 | 95 | 0 |
| 32361 | 232538_at | 0 | 0 | 0 | 0 | 33 |
| 32470 | 232647_at | 0 | 0 | 0 | 0 | 0 |
| 32506 | 232683_s_at | 0 | 0 | 0 | 0 | 0 |
| 32536 | 232713_at | 0 | 92 | 0 | 2 | 0 |
| 32751 | 232929_at | 0 | 0 | 0 | 0 | 0 |
| 32959 | 233137_at | 0 | 0 | 68 | 90 | 0 |
| 32976 | 233154_at | 0 | 0 | 0 | 50 | 0 |
| 32998 | 233176_at | 0 | 0 | 96 | 0 | 0 |
| 33198 | 233378_at | 0 | 0 | 0 | 0 | 10 |
| 33318 | 233498_at | 0 | 52 | 0 | 0 | 0 |
| 33519 | 233699_at | 0 | 0 | 20 | 27 | 0 |
| 33553 | 233733_at | 0 | 0 | 0 | 0 | 0 |
| 33944 | 234125_at | 59 | 0 | 0 | 0 | 0 |
| 34212 | 234394_at | 0 | 37 | 0 | 0 | 0 |
| 34213 | 234395_at | 0 | 0 | 0 | 0 | 0 |
| 34280 | 234462_at | 0 | 0 | 5 | 3 | 0 |
| 34315 | 234497_s_at | 0 | 0 | 0 | 0 | 0 |
| 34333 | 234515_at | 4 | 0 | 0 | 0 | 34 |
| 34396 | 234578_at | 0 | 0 | 0 | 34 | 0 |
| 34491 | 234673_at | 0 | 0 | 0 | 0 | 39 |
| 34497 | 234679_at | 0 | 0 | 0 | 0 | 0 |
| 34503 | 234685_x_at | 0 | 54 | 0 | 0 | 0 |
| 34517 | 234699_at | 0 | 6 | 0 | 0 | 0 |
| 34555 | 234737_at | 0 | 4 | 0 | 0 | 0 |
| 34695 | 234877_x_at | 0 | 0 | 43 | 9 | 0 |
| 34845 | 235027_at | 0 | 0 | 0 | 0 | 0 |
| 34884 | 235066_at | 0 | 0 | 0 | 0 | 0 |
| 34890 | 235072_s_at | 0 | 0 | 33 | 0 | 0 |
| 34896 | 235078_at | 0 | 0 | 0 | 0 | 0 |
| 35065 | 235247_at | 0 | 0 | 0 | 0 | 0 |
| 35189 | 235371_at | 0 | 0 | 0 | 0 | 59 |
| 35268 | 235450_at | 0 | 0 | 0 | 47 | 0 |
| 35410 | 235592_at | 0 | 0 | 72 | 0 | 0 |
| 35564 | 235746_s_at | 0 | 0 | 0 | 85 | 0 |
| 35645 | 235827_at | 0 | 0 | 0 | 0 | 0 |
| 36052 | 236234_at | 0 | 0 | 0 | 0 | 28 |
| 36312 | 236494_x_at | 68 | 0 | 0 | 0 | 0 |
| 36506 | 236688_at | 0 | 0 | 53 | 12 | 80 |
| 36539 | 236721_at | 0 | 0 | 0 | 0 | 0 |
| 36574 | 236756_at | 0 | 0 | 0 | 0 | 0 |
| 36694 | 236876_at | 0 | 0 | 0 | 0 | 24 |
| 36701 | 236883_at | 5 | 16 | 0 | 0 | 77 |
| 36721 | 236903_at | 0 | 0 | 0 | 0 | 0 |
| 36758 | 236940_at | 0 | 0 | 63 | 0 | 0 |
| 36801 | 236983_at | 0 | 0 | 0 | 0 | 50 |
| 37073 | 237255_at | 0 | 0 | 0 | 0 | 0 |
| 37229 | 237411_at | 0 | 0 | 0 | 0 | 0 |
| 37239 | 237421_at | 0 | 0 | 0 | 71 | 0 |
| 37291 | 237473_at | 45 | 0 | 0 | 0 | 0 |
| 37559 | 237741_at | 0 | 0 | 0 | 0 | 0 |
| 37573 | 237755_s_at | 0 | 0 | 0 | 0 | 0 |
| 37590 | 237772_at | 0 | 0 | 62 | 0 | 0 |
| 37630 | 237812_at | 0 | 0 | 0 | 0 | 40 |
| 37647 | 237829_at | 0 | 0 | 0 | 0 | 22 |
| 37649 | 237831_x_at | 0 | 0 | 0 | 0 | 0 |
| 37735 | 237917_at | 0 | 0 | 74 | 0 | 0 |
| 37752 | 237934_at | 0 | 0 | 0 | 0 | 0 |
| 37789 | 237971_at | 0 | 0 | 0 | 0 | 90 |
| 37872 | 238054_at | 25 | 0 | 0 | 0 | 0 |
| 37962 | 238144_s_at | 0 | 0 | 0 | 58 | 0 |
| 37966 | 238148_s_at | 11 | 0 | 0 | 0 | 0 |
| 38158 | 238340_at | 0 | 0 | 0 | 0 | 30 |
| 38289 | 238471_at | 0 | 0 | 0 | 0 | 100 |
| 38445 | 238627_at | 13 | 79 | 0 | 0 | 0 |
| 38536 | 238718_at | 0 | 0 | 0 | 11 | 0 |
| 38756 | 238938_at | 0 | 0 | 0 | 0 | 0 |
| 39035 | 239217_x_at | 0 | 0 | 0 | 0 | 0 |
| 39136 | 239318_at | 0 | 76 | 0 | 0 | 0 |
| 39161 | 239343_at | 0 | 18 | 0 | 0 | 0 |
| 39237 | 239419_at | 0 | 0 | 0 | 0 | 0 |
| 39241 | 239423_at | 0 | 0 | 0 | 45 | 0 |
| 39320 | 239502_at | 0 | 0 | 0 | 0 | 13 |
| 39340 | 239522_at | 0 | 0 | 13 | 0 | 0 |
| 39361 | 239543_s_at | 81 | 82 | 0 | 0 | 0 |
| 39458 | 239640_at | 0 | 0 | 0 | 87 | 0 |
| 39477 | 239659_at | 0 | 0 | 0 | 0 | 86 |
| 39504 | 239686_at | 0 | 0 | 34 | 5 | 0 |
| 39625 | 239807_at | 0 | 0 | 0 | 36 | 0 |
| 39720 | 239902_at | 0 | 0 | 76 | 83 | 0 |
| 39744 | 239926_at | 0 | 0 | 0 | 0 | 0 |
| 39747 | 239929_at | 0 | 0 | 0 | 0 | 0 |
| 39832 | 240014_at | 0 | 0 | 0 | 0 | 49 |
| 40016 | 240198_at | 0 | 0 | 0 | 0 | 94 |
| 40021 | 240203_at | 0 | 0 | 0 | 28 | 0 |
| 40081 | 240263_at | 0 | 0 | 0 | 0 | 29 |
| 40134 | 240316_at | 0 | 0 | 0 | 0 | 0 |
| 40198 | 240380_at | 0 | 69 | 0 | 0 | 0 |
| 40217 | 240399_at | 44 | 57 | 0 | 0 | 0 |
| 40245 | 240427_at | 0 | 0 | 0 | 0 | 92 |
| 40305 | 240487_at | 30 | 0 | 0 | 0 | 0 |
| 40422 | 240604_at | 0 | 0 | 0 | 0 | 0 |
| 40613 | 240795_at | 0 | 0 | 56 | 41 | 0 |
| 40905 | 241087_at | 0 | 0 | 0 | 0 | 0 |
| 40958 | 241140_at | 0 | 0 | 0 | 0 | 0 |
| 41050 | 241232_x_at | 0 | 0 | 0 | 0 | 0 |
| 41149 | 241331_at | 0 | 0 | 0 | 0 | 0 |
| 41215 | 241397_at | 0 | 0 | 0 | 0 | 0 |
| 41302 | 241484_x_at | 0 | 0 | 0 | 0 | 0 |
| 41374 | 241556_at | 0 | 0 | 84 | 0 | 0 |
| 41443 | 241625_at | 0 | 0 | 0 | 0 | 0 |
| 41489 | 241671_x_at | 0 | 0 | 0 | 0 | 0 |
| 41500 | 241682_at | 89 | 0 | 0 | 0 | 0 |
| 41719 | 241901_at | 87 | 0 | 0 | 0 | 0 |
| 41901 | 242083_at | 0 | 0 | 0 | 0 | 0 |
| 41911 | 242093_at | 0 | 0 | 0 | 0 | 0 |
| 42228 | 242410_s_at | 14 | 0 | 0 | 0 | 0 |
| 42244 | 242426_at | 0 | 0 | 30 | 25 | 0 |
| 42287 | 242469_at | 0 | 91 | 0 | 0 | 0 |
| 42333 | 242515_x_at | 0 | 36 | 0 | 0 | 0 |
| 42415 | 242597_at | 0 | 0 | 18 | 57 | 0 |
| 42424 | 242606_at | 0 | 73 | 0 | 0 | 0 |
| 42683 | 242865_at | 0 | 0 | 0 | 0 | 0 |
| 42726 | 242908_x_at | 0 | 0 | 0 | 0 | 72 |
| 42965 | 243147_x_at | 15 | 0 | 0 | 0 | 0 |
| 43009 | 243191_at | 0 | 0 | 0 | 0 | 0 |
| 43041 | 243223_at | 0 | 0 | 0 | 0 | 0 |
| 43100 | 243282_at | 0 | 0 | 0 | 75 | 0 |
| 43210 | 243392_at | 0 | 0 | 0 | 0 | 0 |
| 43219 | 243401_at | 0 | 0 | 0 | 0 | 0 |
| 43415 | 243597_at | 0 | 0 | 0 | 0 | 0 |
| 43420 | 243602_at | 0 | 0 | 0 | 0 | 0 |
| 43565 | 243747_at | 0 | 0 | 0 | 89 | 0 |
| 43618 | 243800_at | 0 | 0 | 0 | 0 | 66 |
| 43777 | 243959_at | 0 | 0 | 0 | 0 | 0 |
| 43889 | 244071_at | 0 | 0 | 0 | 0 | 0 |
| 44334 | 244516_at | 0 | 0 | 0 | 0 | 0 |
| 44407 | 244589_at | 1 | 64 | 0 | 0 | 0 |
| 44411 | 244593_at | 0 | 0 | 0 | 0 | 0 |
| 44451 | 244633_at | 0 | 85 | 0 | 0 | 0 |
| 44582 | 244764_at | 0 | 0 | 0 | 0 | 93 |
| 44584 | 244766_at | 0 | 0 | 64 | 0 | 0 |
| 44717 | 1552266_at | 84 | 0 | 0 | 0 | 0 |
| 44778 | 1557411_s_at | 0 | 0 | 0 | 0 | 0 |
| 44808 | 1552398_a_at | 0 | 0 | 0 | 0 | 0 |
| 45271 | 1553015_a_at | 0 | 0 | 0 | 0 | 0 |
| 45312 | 1553063_at | 0 | 0 | 0 | 0 | 0 |
| 45349 | 1553114_a_at | 41 | 41 | 0 | 0 | 0 |
| 45403 | 1553186_x_at | 0 | 0 | 98 | 0 | 0 |
| 45407 | 1553192_at | 0 | 0 | 67 | 0 | 0 |
| 45430 | 1553222_at | 0 | 0 | 0 | 0 | 0 |
| 45440 | 1553237_x_at | 0 | 0 | 0 | 0 | 0 |
| 45451 | 1553252_a_at | 0 | 0 | 0 | 0 | 45 |
| 45814 | 1553713_a_at | 0 | 0 | 0 | 0 | 0 |
| 45877 | 1553801_a_at | 0 | 0 | 57 | 91 | 0 |
| 45973 | 1553927_at | 0 | 0 | 55 | 0 | 0 |
| 45996 | 1553960_at | 88 | 0 | 0 | 0 | 0 |
| 46126 | 1554147_s_at | 27 | 0 | 0 | 0 | 0 |
| 46539 | 1554710_at | 0 | 0 | 0 | 0 | 0 |
| 46892 | 1555181_a_at | 0 | 0 | 0 | 0 | 69 |
| 47027 | 1555356_a_at | 0 | 38 | 0 | 0 | 0 |
| 47038 | 1555368_x_at | 0 | 0 | 0 | 0 | 0 |
| 47085 | 1555431_a_at | 50 | 0 | 0 | 0 | 0 |
| 47106 | 1555462_at | 0 | 0 | 0 | 0 | 15 |
| 47115 | 1555472_at | 0 | 2 | 0 | 0 | 0 |
| 47298 | 1555754_s_at | 0 | 35 | 0 | 0 | 0 |
| 47323 | 1555786_s_at | 0 | 0 | 0 | 0 | 8 |
| 47374 | 1555853_at | 0 | 77 | 0 | 0 | 0 |
| 47470 | 1555990_at | 0 | 0 | 0 | 93 | 0 |
| 47482 | 1556006_s_at | 0 | 0 | 0 | 0 | 0 |
| 47691 | 1556336_at | 0 | 86 | 0 | 0 | 0 |
| 47784 | 1556471_at | 0 | 40 | 0 | 0 | 0 |
| 47858 | 1556597_a_at | 0 | 0 | 0 | 0 | 0 |
| 47868 | 1556616_a_at | 0 | 0 | 0 | 0 | 0 |
| 47897 | 1556655_s_at | 55 | 13 | 0 | 0 | 0 |
| 47925 | 1556695_a_at | 0 | 0 | 0 | 0 | 0 |
| 47926 | 1556696_s_at | 0 | 0 | 0 | 0 | 54 |
| 47941 | 1556717_at | 0 | 0 | 0 | 70 | 0 |
| 48143 | 1557012_a_at | 0 | 47 | 0 | 0 | 0 |
| 48159 | 1557038_s_at | 0 | 0 | 59 | 0 | 0 |
| 48237 | 1557167_at | 0 | 0 | 0 | 0 | 84 |
| 48265 | 1557217_a_at | 0 | 0 | 0 | 0 | 91 |
| 48343 | 1557343_at | 0 | 24 | 0 | 0 | 0 |
| 48404 | 1557437_a_at | 0 | 0 | 60 | 0 | 0 |
| 48540 | 1557633_at | 0 | 0 | 0 | 0 | 51 |
| 48544 | 1557639_at | 0 | 94 | 0 | 0 | 0 |
| 48783 | 1558017_s_at | 0 | 0 | 0 | 0 | 0 |
| 48789 | 1558034_s_at | 0 | 0 | 0 | 0 | 0 |
| 48977 | 1558438_a_at | 0 | 0 | 0 | 0 | 0 |
| 49079 | 1558624_at | 0 | 0 | 0 | 0 | 12 |
| 49168 | 1558770_a_at | 0 | 17 | 0 | 0 | 0 |
| 49203 | 1558822_at | 0 | 0 | 0 | 0 | 0 |
| 49237 | 1558875_at | 0 | 0 | 10 | 48 | 0 |
| 49250 | 1558897_at | 0 | 25 | 0 | 0 | 0 |
| 49357 | 1559086_at | 0 | 0 | 0 | 0 | 0 |
| 49377 | 1559124_at | 0 | 0 | 0 | 0 | 0 |
| 49385 | 1559133_at | 0 | 0 | 77 | 76 | 0 |
| 49404 | 1559163_at | 0 | 0 | 0 | 0 | 0 |
| 49429 | 1559218_s_at | 0 | 0 | 0 | 0 | 87 |
| 49714 | 1559686_a_at | 0 | 0 | 0 | 0 | 0 |
| 49727 | 1559706_at | 70 | 65 | 0 | 0 | 98 |
| 49761 | 1559771_at | 0 | 0 | 100 | 0 | 0 |
| 49797 | 1559848_at | 0 | 0 | 15 | 0 | 0 |
| 49967 | 1560144_at | 0 | 0 | 0 | 0 | 0 |
| 50106 | 1560405_at | 0 | 0 | 0 | 0 | 56 |
| 50574 | 1561154_at | 0 | 0 | 0 | 0 | 0 |
| 50602 | 1561200_at | 0 | 0 | 0 | 0 | 0 |
| 50640 | 1561247_at | 0 | 0 | 0 | 56 | 0 |
| 50678 | 1561294_a_at | 0 | 19 | 0 | 0 | 0 |
| 50696 | 1561322_at | 0 | 99 | 0 | 0 | 0 |
| 50722 | 1569452_at | 0 | 0 | 0 | 0 | 0 |
| 50754 | 1561407_at | 0 | 0 | 0 | 0 | 0 |
| 50833 | 1561511_at | 0 | 0 | 0 | 0 | 74 |
| 50955 | 1561683_at | 0 | 23 | 0 | 0 | 0 |
| 50987 | 1561739_at | 0 | 97 | 0 | 0 | 0 |
| 51032 | 1561868_at | 0 | 45 | 0 | 0 | 0 |
| 51135 | 1562035_at | 0 | 0 | 50 | 0 | 0 |
| 51177 | 1562099_at | 0 | 51 | 0 | 0 | 0 |
| 51229 | 1562230_at | 0 | 0 | 23 | 43 | 0 |
| 51342 | 1562412_at | 0 | 0 | 0 | 0 | 0 |
| 51588 | 1562801_at | 0 | 0 | 0 | 0 | 0 |
| 51611 | 1562836_at | 0 | 0 | 0 | 0 | 0 |
| 51618 | 1562849_at | 52 | 0 | 0 | 0 | 0 |
| 51639 | 1562880_at | 0 | 0 | 0 | 0 | 68 |
| 51802 | 1563106_at | 0 | 0 | 0 | 0 | 0 |
| 51928 | 1563348_at | 0 | 0 | 0 | 0 | 0 |
| 51940 | 1563376_at | 58 | 0 | 0 | 0 | 0 |
| 51960 | 1563450_at | 0 | 66 | 0 | 0 | 0 |
| 51981 | 1563478_at | 0 | 0 | 71 | 0 | 0 |
| 52033 | 1563581_at | 0 | 0 | 0 | 0 | 0 |
| 52052 | 1563614_at | 0 | 0 | 88 | 0 | 0 |
| 52063 | 1563655_at | 7 | 0 | 0 | 0 | 0 |
| 52114 | 1563800_at | 42 | 0 | 0 | 0 | 0 |
| 52135 | 1563853_at | 0 | 0 | 8 | 81 | 0 |
| 52354 | 1564402_at | 0 | 0 | 31 | 68 | 0 |
| 52414 | 1564600_a_at | 0 | 0 | 0 | 0 | 0 |
| 52547 | 1565065_at | 0 | 0 | 0 | 0 | 25 |
| 52828 | 1565935_at | 0 | 0 | 0 | 0 | 0 |
| 52968 | 1566294_at | 72 | 0 | 0 | 0 | 0 |
| 52985 | 1566425_at | 0 | 0 | 0 | 0 | 0 |
| 53014 | 1566469_at | 0 | 0 | 0 | 0 | 63 |
| 53063 | 1566550_at | 0 | 0 | 0 | 0 | 99 |
| 53125 | 1566698_at | 0 | 95 | 0 | 0 | 0 |
| 53155 | 1566775_at | 0 | 0 | 0 | 0 | 0 |
| 53225 | 1566902_at | 0 | 7 | 0 | 0 | 0 |
| 53309 | 1567079_at | 0 | 29 | 0 | 0 | 0 |
| 53332 | 1567240_x_at | 0 | 0 | 0 | 0 | 0 |
| 53339 | 1567248_at | 2 | 0 | 0 | 0 | 0 |
| 53614 | 1568854_at | 0 | 0 | 0 | 22 | 0 |
| 53702 | 1569009_s_at | 0 | 0 | 0 | 0 | 0 |
| 53707 | 1569023_a_at | 0 | 0 | 0 | 0 | 0 |
| 53860 | 1569318_at | 0 | 0 | 0 | 0 | 0 |
| 54119 | 1569755_at | 17 | 0 | 0 | 0 | 0 |
| 54150 | 1569793_at | 0 | 0 | 75 | 0 | 0 |
| 54206 | 1569882_at | 0 | 0 | 0 | 0 | 47 |
| 54295 | 1570033_at | 0 | 0 | 0 | 0 | 0 |
| 54482 | 1570318_at | 0 | 62 | 0 | 0 | 0 |
| 54485 | 1570327_at | 0 | 0 | 0 | 0 | 0 |
| 54541 | 1570433_at | 0 | 100 | 0 | 0 | 0 |

| | | | | Average | | Accession |
|---|---|---|---|---|---|---|
| N1:54630 | DAS28wk16_70Dn3 | cDAS28_110Dn3 | cDAS28_80Dn3 | Score | Gene Symbol | Number |
| 91 | 0 | 0 | 0 | 7 | SPAG7 | NM_004890 |
| 145 | 0 | 0 | 0 | 3 | MSN | NM_002444 |
| 210 | 0 | 0 | 0 | 7.5 | SPARC | NM_003118 |
| 495 | 0 | 0 | 0 | 2.5 | ARPC1A | NM_006409 |
| 523 | 0 | 0 | 0 | 5.625 | MDH1 | NM_005917 |
| 617 | 0 | 0 | 0 | 2.25 | SMARCC1 | AW152160 |
| 670 | 0 | 0 | 0 | 18.75 | ITGB5 | NM_002213 |
| 708 | 0 | 0 | 79 | 2.75 | IGFBP7 | NM_001553 |
| 771 | 0 | 0 | 0 | 8.5 | NDUFB8 /// | NM_005004 |
| | | | | | SEC31B | |
| 846 | 0 | 0 | 0 | 2 | ANXA4 | BC000182 |
| 915 | 0 | 0 | 30 | 8.875 | CUL3 | AU145232 |
| 931 | 0 | 15 | 0 | 10.75 | DHX15 | AF279891 |
| 962 | 0 | 47 | 0 | 6.75 | SOX4 | AL136179 |
| 1005 | 0 | 0 | 0 | 9.375 | MAPKAPK2 | AI141802 |
| 1007 | 0 | 0 | 0 | 5.625 | SCRN1 | NM_014766 |
| 1013 | 0 | 0 | 0 | 1.875 | NQO1 | NM_000903 |
| 1056 | 0 | 21 | 0 | 10 | AAMP | NM_001087 |
| 1081 | 43 | 0 | 0 | 7.25 | DUSP3 | AL048503 |
| 1226 | 0 | 0 | 0 | 1.5 | DLG5 | AB011155 |
| 1429 | 0 | 0 | 0 | 3.5 | CEACAM5 | NM_004363 |
| 1450 | 0 | 0 | 98 | 0.375 | CTDSPL | BF590317 |
| 1456 | 78 | 0 | 0 | 2.875 | FARP1 | NM_005766 |
| 1519 | 0 | 0 | 0 | 3.75 | C7orf28A | NM_015622 |
| 1528 | 0 | 23 | 78 | 12.625 | EGFR | AW157070 |
| 1581 | 0 | 0 | 0 | 6 | SFRP1 | AF017987 |
| 1686 | 0 | 50 | 0 | 6.375 | COPS8 | BC003090 |
| 1869 | 0 | 65 | 0 | 4.5 | ACBD3 | NM_022735 |
| 1910 | 0 | 0 | 0 | 6.625 | UNC119B | BC004815 |
| 2062 | 0 | 0 | 0 | 0.5 | CRMP1 | NM_001313 |
| 2124 | 0 | 0 | 0 | 9.125 | HMGN4 | NM_006353 |
| 2235 | 37 | 0 | 0 | 8 | SNRPD1 | BC001721 |
| 2327 | 0 | 0 | 0 | 1.25 | INPP5K | NM_016532 |
| 2411 | 0 | 0 | 0 | 18.625 | DNAJB12 | BG283782 |
| 2614 | 0 | 0 | 0 | 11.5 | KLHL21 | NM_014851 |
| 2717 | 47 | 0 | 0 | 6.75 | FXR2 | NM_004860 |
| 2767 | 0 | 86 | 0 | 1.875 | TLE1 | NM_005077 |
| 2815 | 0 | 0 | 0 | 0.75 | UNC119 | NM_005148 |
| 2823 | 0 | 0 | 0 | 9.25 | EDEM1 | NM_014674 |
| 2837 | 0 | 0 | 0 | 10.875 | LMAN1 | NM_005570 |
| 2997 | 100 | 0 | 0 | 0.125 | SCNN1A | NM_001038 |
| 3131 | 0 | 0 | 0 | 5.375 | ARL4D | U25771 |
| 3435 | 0 | 0 | 0 | 7.375 | DAPK3 | NM_001348 |
| 3446 | 30 | 0 | 0 | 8.875 | HEPH | AU148222 |
| 3455 | 0 | 0 | 35 | 8.25 | RAP1GAP | NM_002885 |
| 3511 | 49 | 0 | 0 | 6.5 | CDC6 | U77949 |
| 3585 | 0 | 0 | 0 | 10.5 | MAOB | NM_000898 |
| 3661 | 0 | 0 | 0 | 4.25 | PREP | NM_002726 |
| 3937 | 0 | 0 | 0 | 4.125 | ACPP | NM_001099 |
| 3942 | 0 | 0 | 0 | 9.375 | EML2 | NM_012155 |
| 3966 | 0 | 0 | 0 | 3.125 | FGF2 | NM_002006 |
| 3982 | 0 | 0 | 0 | 4.75 | MRC1 /// | NM_002438 |
| | | | | | MRC1L1 | |
| 4000 | 0 | 0 | 34 | 8.375 | GAS1 | AW611727 |
| 4171 | 0 | 0 | 0 | 12.25 | ITGB3 | M35999 |
| 4316 | 81 | 0 | 0 | 14.375 | TTF1 | NM_007344 |
| 4346 | 41 | 0 | 0 | 7.5 | RRAD | NM_004165 |
| 4493 | 0 | 0 | 0 | 10.5 | ICAM3 | NM_002162 |
| 4497 | 0 | 0 | 0 | 8 | SNAP91 | NM_014841 |
| 4506 | 0 | 0 | 0 | 12.625 | CENPA | NM_001809 |
| 4528 | 0 | 0 | 0 | 10.75 | GPC4 | NM_001448 |
| 4805 | 34 | 0 | 0 | 17 | PGC | NM_002630 |
| 4825 | 0 | 39 | 8 | 22.125 | PIGA | NM_002641 |
| 4838 | 77 | 0 | 0 | 3 | BAIAP2 | NM_017450 |
| 4976 | 90 | 0 | 0 | 1.375 | OVGP1 | NM_002557 |
| 5175 | 0 | 85 | 27 | 22.875 | KIAA0586 | NM_014749 |
| 5405 | 91 | 0 | 0 | 1.25 | SPIB | NM_003121 |
| 5414 | 0 | 0 | 0 | 5.125 | BDKRB2 | NM_000623 |
| 5536 | 0 | 0 | 43 | 7.25 | IL15 | NM_000585 |
| 5622 | 0 | 0 | 0 | 4.625 | CHML | NM_001821 |
| 5689 | 0 | 0 | 0 | 2.375 | RHAG | AF178841 |
| 5868 | 0 | 0 | 0 | 4.875 | SERPINA6 | NM_001756 |
| 5970 | 0 | 0 | 99 | 0.25 | MLANA | U06654 |
| 6036 | 0 | 0 | 0 | 18.75 | ITGA2B | NM_000419 |
| 6046 | 0 | 0 | 0 | 0.25 | PML | NM_002675 |
| 6104 | 0 | 77 | 0 | 3 | AKR1B10 | NM_020299 |
| 6116 | 0 | 0 | 0 | 0.375 | KCNQ3 | NM_004519 |
| 6152 | 0 | 0 | 0 | 12 | MAGEC1 | NM_005462 |
| 6177 | 0 | 0 | 0 | 7.375 | SIX3 | NM_005413 |
| 6178 | 0 | 0 | 100 | 0.125 | CHRNB2 | NM_000748 |
| 6254 | 0 | 0 | 0 | 5.5 | CXorf1 | NM_004709 |
| 6273 | 0 | 0 | 0 | 24.75 | GRIA3 | NM_007325 |
| 6341 | 11 | 0 | 0 | 11.25 | DLEC1 | NM_005106 |
| 6495 | 0 | 0 | 0 | 12.125 | G6PC | NM_000151 |
| 6637 | 0 | 0 | 0 | 2.625 | IL8RA | NM_000634 |
| 6639 | 26 | 0 | 0 | 11.625 | SAA4 | NM_006512 |
| 6777 | 0 | 0 | 0 | 12.125 | GRM5 | NM_000842 |
| 6869 | 0 | 0 | 0 | 3.625 | ALOX15 | NM_001140 |
| 6905 | 0 | 0 | 0 | 2.25 | USP34 | NM_014709 |
| 6968 | 57 | 0 | 0 | 12.375 | SLC22A2 | NM_003058 |
| 7108 | 0 | 0 | 0 | 12 | SHOX | NM_000451 |
| 7136 | 0 | 0 | 0 | 5.125 | XRCC2 | NM_005431 |
| 7201 | 97 | 0 | 0 | 0.5 | GAGE3 | NM_001473 |
| 7231 | 0 | 0 | 0 | 2.875 | CACNB4 | NM_000726 |
| 7268 | 0 | 0 | 0 | 6 | — | NM_017932 |
| 7353 | 0 | 76 | 49 | 9.625 | IFNW1 | NM_002177 |
| 7400 | 0 | 0 | 0 | 6.875 | SCN7A | NM_002976 |
| 7545 | 0 | 0 | 0 | 2.375 | ZNF157 | NM_003446 |
| 7634 | 0 | 4 | 42 | 19.5 | AVPR2 | AF030626 |
| 7709 | 0 | 0 | 86 | 1.875 | LIPE | NM_005357 |
| 7731 | 0 | 0 | 0 | 9.75 | C4BPB | NM_000716 |
| 7749 | 0 | 0 | 0 | 1.875 | ADAM22 | NM_021721 |
| 7812 | 68 | 0 | 0 | 4.125 | TH | NM_000360 |
| 7901 | 0 | 0 | 85 | 2 | PCK1 | NM_002591 |
| 7991 | 0 | 0 | 70 | 3.875 | FRMD4A | NM_018027 |
| 8286 | 0 | 0 | 0 | 3.75 | CSNK1D | AV700224 |
| 8401 | 72 | 0 | 0 | 3.625 | NCOR2 | AI373205 |
| 8631 | 0 | 0 | 0 | 2.75 | NR2F2 | AL037401 |
| 8799 | 0 | 37 | 0 | 8 | CDC42EP3 | AL136842 |
| 8874 | 0 | 0 | 0 | 1.625 | BAD | U66879 |
| 9068 | 0 | 72 | 69 | 7.625 | HIP1R | AB013384 |
| 9157 | 0 | 0 | 55 | 5.75 | TGFB1I1 | BC001830 |
| 9240 | 0 | 0 | 0 | 2.625 | ABCG2 | AF098951 |
| 9296 | 0 | 0 | 0 | 12.375 | PADI2 | AL049569 |
| 9337 | 0 | 0 | 0 | 5.75 | CHST3 | AB017915 |
| 9362 | 0 | 0 | 0 | 1.125 | TRIM9 | AF220036 |
| 9525 | 0 | 0 | 0 | 4.375 | PCGF1 | BC004952 |
| 9541 | 19 | 0 | 0 | 20.125 | PRKCQ | L01087 |
| 9596 | 85 | 0 | 0 | 2 | IGFBP3 | M31159 |
| 9633 | 0 | 69 | 88 | 5.625 | EFNA3 | AW189015 |
| 9696 | 0 | 0 | 0 | 9.125 | PSG1 | M34715 |
| 9728 | 0 | 0 | 0 | 2 | — | BC003629 |
| 9742 | 0 | 0 | 0 | 3.25 | ABCC8 | L78207 |
| 9766 | 0 | 53 | 0 | 6 | SFRS17A | M99578 |
| 9826 | 42 | 0 | 0 | 11.125 | SGCD | U58331 |
| 9915 | 10 | 0 | 0 | 36.75 | SLC24A1 | AB014602 |
| 10007 | 0 | 0 | 0 | 5.25 | FETUB | AB017551 |
| 10093 | 0 | 0 | 0 | 18.375 | DTNA | U26744 |
| 10143 | 62 | 0 | 0 | 4.875 | GLRA3 | U93917 |
| 10447 | 0 | 0 | 0 | 25 | MCM7 | AF279900 |
| 10489 | 0 | 36 | 3 | 20.375 | MGLL | BC006230 |
| 10757 | 0 | 31 | 0 | 8.75 | PTPRU | U71075 |
| 10773 | 0 | 0 | 0 | 7.5 | IFNA2 | M54886 |
| 10797 | 0 | 0 | 0 | 3.875 | MTAP | AF109294 |
| 10884 | 0 | 0 | 0 | 4.75 | GABARAPL1 | AF180519 |
| | | | | | /// | |
| | | | | | GABARAPL3 | |
| 10893 | 0 | 0 | 0 | 12 | NFIB | U70862 |
| 10895 | 0 | 0 | 0 | 7.25 | CXCR6 | U73531 |
| 10992 | 0 | 0 | 75 | 3.25 | RAPSN | BC004196 |
| 11058 | 0 | 0 | 0 | 0.5 | IGH@ /// | 0 |
| | | | | | IGHA1 /// | |
| | | | | | IGHA2 /// | |
| | | | | | IGHD /// | |
| | | | | | IGHG1 /// | |
| | | | | | IGHG3 /// | |
| | | | | | IGHG4 /// | |
| | | | | | IGHM /// | |
| | | | | | IGHV3-23 /// | |
| | | | | | IGHV4-31 /// | |
| | | | | | LOC100126583 | |
| | | | | | /// | |
| | | | | | LOC642131 | |
| | | | | | /// | |
| | | | | | LOC652128 | |
| | | | | | /// VSIG6 | |
| 11061 | 0 | 0 | 0 | 14 | IGHA1 /// | L23519 |
| | | | | | IGHG1 /// | |
| | | | | | LOC100133862 | |
| 11240 | 0 | 0 | 0 | 12.25 | KCND3 | AF187964 |
| 11328 | 2 | 0 | 0 | 41.625 | ZNF471 | AF352026 |
| 11495 | 0 | 0 | 0 | 2.375 | PEG10 | BE858180 |
| 11500 | 32 | 0 | 37 | 25 | CAV1 | AU147399 |
| 11645 | 0 | 0 | 0 | 8 | TUBA4A | AL565074 |
| 11867 | 0 | 0 | 0 | 6.875 | SETD3 | AA524500 |
| 11918 | 0 | 0 | 0 | 7.875 | ARAP1 | AB018325 |
| 11930 | 0 | 0 | 0 | 3.5 | — | AI348009 |
| 12160 | 0 | 0 | 0 | 26 | ZEB1 | AI373166 |
| 12199 | 0 | 0 | 0 | 5.375 | SORT1 | BE742268 |
| 12294 | 0 | 0 | 72 | 3.625 | ZNF282 | AW130128 |
| 12391 | 0 | 0 | 0 | 1.375 | FBXO9 | AL137520 |
| 12680 | 0 | 1 | 4 | 24.625 | APOOL | BE501952 |
| 12698 | 0 | 0 | 0 | 2.25 | ATG2A | AW168132 |
| 12721 | 0 | 0 | 0 | 10.5 | ZC3H7B | BE855831 |
| 12750 | 0 | 0 | 0 | 4.875 | TMCC1 | AB018322 |
| 12820 | 0 | 0 | 0 | 21.875 | MXRA8 | AW888223 |
| 13420 | 0 | 0 | 0 | 3.375 | DGCR6L | AA631156 |
| 13433 | 0 | 0 | 0 | 15.625 | CCDC22 | BF224247 |
| 13576 | 0 | 0 | 0 | 11 | MAN1C1 | AW340588 |
| 13717 | 0 | 0 | 0 | 14.875 | NOV | BF440025 |
| 13806 | 0 | 0 | 90 | 1.375 | TRPM1 | N32151 |
| 13834 | 0 | 0 | 0 | 6.625 | HLX | M60721 |
| 13970 | 0 | 0 | 0 | 12.25 | AZU1 | NM_001700 |
| 14067 | 17 | 0 | 0 | 11 | USP19 | AW451502 |
| 14135 | 0 | 11 | 29 | 20.25 | AZI1 | AB029041 |
| 14159 | 0 | 0 | 0 | 9.125 | AHCTF1 | AL080144 |
| 14162 | 0 | 0 | 0 | 9.5 | CLCN4 | AF052117 |
| 14170 | 0 | 0 | 0 | 5 | IGKV4-1 | BG482805 |
| 14213 | 0 | 10 | 0 | 11.375 | — | AF052119 |
| 14276 | 0 | 44 | 0 | 7.125 | MCF2 | AL033403 |
| 14290 | 0 | 0 | 0 | 9.25 | MUC3B | AB038783 |
| 14350 | 23 | 0 | 0 | 26.75 | TMC6 | AK021738 |
| 14398 | 0 | 0 | 0 | 0.875 | — | AK023816 |
| 14407 | 0 | 0 | 0 | 10.875 | DST | BC004912 |
| 14446 | 0 | 82 | 5 | 14.375 | B3GNTL1 | U79265 |
| 14581 | 48 | 0 | 0 | 6.625 | EIF3M | AV717062 |
| 14642 | 0 | 7 | 0 | 11.75 | — | AA595276 |
| 14913 | 0 | 0 | 0 | 3.5 | ZNF391 | AL031118 |
| 14980 | 0 | 0 | 0 | 7.125 | LOC100128640 | AK025619 |
| 14994 | 0 | 0 | 0 | 10.375 | — | AK023783 |
| 15045 | 60 | 0 | 0 | 5.125 | GRIK2 | AU156204 |
| 15099 | 0 | 0 | 0 | 0.125 | LOC100134355 | AL121975 |
| | | | | | /// PRIM2 | |
| 15122 | 0 | 0 | 0 | 7 | DTX2 /// | AK023924 |
| | | | | | LOC100134197 | |
| 15127 | 0 | 0 | 0 | 6.875 | USF2 | X90824 |
| 15233 | 0 | 0 | 0 | 16.5 | TLL2 | AK026106 |
| 15309 | 0 | 56 | 18 | 16 | MRPS11 | BC000200 |
| 15525 | 65 | 0 | 0 | 4.5 | — | AF113683 |
| 15612 | 0 | 0 | 0 | 9.375 | CPN2 | J05158 |
| 15618 | 0 | 0 | 0 | 10.25 | HCG2P7 | X81001 |
| 15818 | 0 | 0 | 0 | 6.125 | IGL@ | AF043586 |
| 15871 | 0 | 0 | 0 | 11.625 | C19orf10 | AC005339 |
| 15953 | 0 | 0 | 0 | 20.25 | — | D84140 |
| 16315 | 0 | 74 | 0 | 3.375 | TAL1 | X51990 |
| 16393 | 0 | 0 | 0 | 5.75 | FASN | U52428 |
| 16398 | 20 | 0 | 0 | 22.875 | GBX1 | L11239 |
| 16421 | 0 | 0 | 0 | 3 | NTN3 | AF103529 |
| 16510 | 7 | 0 | 0 | 23.875 | — | K00627 |
| 16521 | 0 | 0 | 0 | 21.75 | — | K00627 |
| 16547 | 0 | 38 | 0 | 7.875 | ESR1 | X63118 |
| 16560 | 0 | 0 | 0 | 9.125 | ZFX | X59740 |
| 16584 | 0 | 45 | 0 | 7 | CYB561 | U06715 |
| 16606 | 0 | 0 | 0 | 4.5 | LOC642131 | S74639 |
| 16673 | 0 | 13 | 0 | 11 | CEACAM5 | Z21818 |
| 16686 | 0 | 0 | 13 | 11 | SHMT1 | Y14488 |
| 16863 | 0 | 0 | 0 | 2 | — | AL110201 |
| 16865 | 0 | 0 | 0 | 11.75 | FOLH1 | AF254357 |
| 16922 | 1 | 0 | 0 | 24 | FAM55C | AA721025 |
| 16961 | 0 | 0 | 0 | 0.625 | — | AW301806 |
| 17051 | 0 | 0 | 0 | 4 | AKAP6 | AW451230 |
| 17091 | 0 | 42 | 0 | 7.375 | — | AV647366 |
| 17248 | 0 | 0 | 0 | 8.375 | CPSF7 | NM_024811 |
| 17294 | 0 | 0 | 0 | 7.375 | DUS1L | NM_022156 |
| 17514 | 0 | 0 | 0 | 9 | TSEN34 | NM_024075 |
| 17526 | 0 | 0 | 82 | 2.375 | INF2 | NM_022489 |
| 17680 | 0 | 92 | 0 | 1.125 | C14orf159 | NM_024952 |
| 17736 | 0 | 0 | 0 | 6 | TRAPPC2L | NM_016209 |
| 17757 | 25 | 0 | 0 | 9.5 | NUDT9 | NM_024047 |
| 17785 | 0 | 99 | 0 | 0.25 | TRIAP1 | NM_016399 |
| 17803 | 0 | 0 | 0 | 11.125 | CERK | NM_022766 |
| 17821 | 0 | 0 | 0 | 0.625 | COMMD10 | NM_016144 |
| 17943 | 0 | 0 | 0 | 12.25 | LYRM4 | NM_020408 |
| 17955 | 0 | 91 | 0 | 1.25 | MAGEH1 | NM_014061 |
| 17959 | 0 | 9 | 12 | 22.625 | LRRC40 | NM_017768 |
| 18051 | 0 | 0 | 0 | 16.5 | PUS1 | NM_025215 |
| 18066 | 0 | 28 | 93 | 10.125 | SMUG1 | NM_014311 |
| 18074 | 0 | 0 | 0 | 8.25 | TSPAN15 | NM_012339 |
| 18196 | 0 | 0 | 0 | 5 | TMEM51 | NM_018022 |
| 18263 | 0 | 75 | 0 | 3.25 | WDR3 | NM_006784 |
| 18295 | 0 | 0 | 0 | 2.625 | C1orf66 | NM_015997 |
| 18325 | 0 | 0 | 0 | 2.5 | PYCRL | NM_023078 |
| 18344 | 0 | 0 | 0 | 8 | KRT23 | NM_015515 |
| 18474 | 0 | 3 | 9 | 23.75 | PID1 | NM_017933 |
| 18663 | 0 | 0 | 0 | 5 | TRPV2 | NM_015930 |
| 18692 | 0 | 0 | 0 | 4.875 | CEP76 | NM_024899 |
| 18790 | 0 | 0 | 0 | 11.5 | SNIP1 | NM_024700 |
| 18870 | 0 | 0 | 54 | 5.875 | NXN | NM_017821 |
| 18930 | 0 | 0 | 0 | 3.875 | RTN3 | NM_006054 |
| 18946 | 0 | 73 | 0 | 3.5 | CYP20A1 | NM_020674 |
| 19008 | 0 | 0 | 0 | 2.375 | ZNF767 | NM_024910 |
| 19024 | 0 | 6 | 51 | 18.125 | LRP1B | NM_018557 |
| 19452 | 0 | 0 | 0 | 10 | HAUS2 | NM_018097 |
| 19474 | 0 | 0 | 0 | 3.5 | ANTXR1 | NM_018153 |
| 19680 | 0 | 14 | 0 | 10.875 | SPATA6 | NM_019073 |
| 19733 | 0 | 0 | 0 | 9.75 | FLJ42627 | NM_024305 |
| 19837 | 0 | 0 | 0 | 5.875 | SPTLC3 | NM_018327 |
| 19887 | 0 | 49 | 0 | 6.5 | GUCY1B2 | NM_004129 |
| 19973 | 0 | 0 | 0 | 0.375 | CCDC40 | NM_017950 |
| 20125 | 0 | 0 | 50 | 6.375 | IFT122 | NM_018262 |
| 20192 | 52 | 0 | 0 | 6.125 | PRG3 | NM_006093 |
| 20209 | 0 | 0 | 0 | 0.375 | FLJ11292 | NM_018382 |
| 20214 | 0 | 0 | 0 | 1 | — | NM_016241 |
| 20220 | 0 | 0 | 0 | 3.25 | METTL5 | NM_014168 |
| 20258 | 0 | 0 | 0 | 8.125 | — | NM_018580 |
| 20390 | 51 | 0 | 0 | 6.25 | ANGPTL4 | NM_016109 |
| 20401 | 0 | 0 | 0 | 8.75 | SLC25A32 | NM_030780 |
| 20424 | 0 | 0 | 0 | 2.5 | — | NM_013395 |
| 20512 | 0 | 40 | 0 | 7.625 | CLDN18 | NM_016369 |
| 20547 | 0 | 0 | 0 | 6 | CCDC70 | NM_031290 |
| 20549 | 0 | 0 | 0 | 7.125 | HRH4 | NM_021624 |
| 20690 | 0 | 0 | 0 | 9.375 | FGF14 | NM_004115 |
| 20752 | 0 | 0 | 0 | 19.375 | P2RX2 | NM_012226 |
| 20788 | 86 | 0 | 0 | 1.875 | PCDHB12 | NM_018932 |
| 20816 | 0 | 0 | 0 | 10.25 | CDCA3 | NM_031299 |
| 20956 | 0 | 0 | 0 | 7.25 | GDF15 | AF003934 |
| 21196 | 0 | 0 | 0 | 6.75 | RAB35 | BF791960 |
| 21238 | 0 | 0 | 0 | 23.5 | — | AL157484 |
| 21263 | 0 | 0 | 26 | 9.375 | DENND2A | AL037701 |
| 21281 | 0 | 0 | 0 | 4.25 | FAM131A | AI141670 |
| 21648 | 0 | 0 | 0 | 1.375 | SCIN | BG283584 |
| 21652 | 0 | 0 | 0 | 7.375 | — | AA837026 |
| 21833 | 0 | 0 | 0 | 3.375 | KCTD2 | D79998 |
| 21843 | 0 | 0 | 0 | 16.5 | FXR2 | AF044263 |
| 21925 | 0 | 0 | 0 | 18.375 | ARHGAP25 | D29642 |
| 21974 | 0 | 0 | 0 | 8 | STK10 | AB015718 |
| 22102 | 64 | 0 | 0 | 4.625 | KIAA1644 | AL047020 |
| 22287 | 0 | 0 | 0 | 6.25 | THRAP3 | BE967048 |
| 22352 | 0 | 0 | 53 | 6 | COX4NB | BC001472 |
| 22628 | 0 | 0 | 0 | 8.5 | BAALC | AI870583 |
| 22742 | 0 | 0 | 0 | 13.625 | C20orf7 | AI640582 |
| 23095 | 0 | 0 | 0 | 11.875 | CLDN12 | AL136770 |
| 23127 | 0 | 0 | 0 | 1.5 | COX15 | AF026850 |
| 23130 | 0 | 0 | 0 | 8.25 | NAT14 | AB038651 |
| 23337 | 0 | 22 | 0 | 9.875 | COMMD2 | BC001228 |
| 23353 | 0 | 71 | 0 | 3.75 | CLPX | AL136922 |
| 23368 | 0 | 0 | 0 | 7.125 | TMEM108 | BC000568 |
| 23788 | 0 | 0 | 0 | 11.375 | NLRP12 | AF231021 |
| 23830 | 0 | 0 | 83 | 2.25 | CHRDL2 | AF332891 |
| 23870 | 98 | 0 | 0 | 0.375 | CCL28 | AF110384 |
| 23914 | 31 | 0 | 0 | 8.75 | IL20 | AF224266 |
| 23943 | 0 | 0 | 0 | 3.875 | DPYSL5 | BC002874 |
| 24025 | 0 | 0 | 0 | 8.875 | BOC | AY027658 |
| 24093 | 0 | 0 | 0 | 4.375 | — | AF116695 |
| 24121 | 0 | 0 | 0 | 15.75 | FKSG49 | AF338193 |
| 24187 | 0 | 0 | 0 | 6.875 | LOC100131508 | AF130064 |
| 24310 | 0 | 0 | 0 | 1.5 | AGPAT9 | BC006236 |
| 24359 | 0 | 0 | 0 | 11.125 | NT5C1A | AY028778 |
| 24369 | 0 | 0 | 0 | 4.625 | PCDHAC2 | AF152474 |
| 24463 | 0 | 5 | 56 | 17.625 | BIRC6 | AI017106 |
| 24489 | 0 | 0 | 0 | 10.5 | PIGY | AW028485 |
| 24610 | 56 | 0 | 0 | 15.5 | FAM100B | AA831661 |
| 24619 | 0 | 0 | 0 | 9.625 | TNKS1BP1 | AL566438 |
| 24752 | 0 | 0 | 0 | 2.875 | PREX1 | AL445192 |
| 24753 | 0 | 0 | 0 | 8.25 | EXOC4 | AB051486 |
| 24828 | 0 | 0 | 0 | 15.5 | RAB3D | AI744658 |
| 24903 | 83 | 0 | 0 | 2.25 | CHD2 | AA890703 |
| 25062 | 0 | 0 | 20 | 21.375 | RBM18 | AA167623 |
| 25121 | 0 | 60 | 32 | 13.75 | SLC39A10 | AB033091 |
| 25156 | 0 | 0 | 0 | 4.25 | IGF1R | AL044092 |
| 25197 | 0 | 0 | 0 | 10.875 | GLE1 | AI638714 |
| 25312 | 0 | 0 | 0 | 11.875 | ARID2 | AB046777 |
| 25404 | 0 | 89 | 0 | 1.5 | C13orf37 | AI885466 |
| 25461 | 0 | 78 | 57 | 8.375 | LOC401504 | BG535378 |
| 25669 | 0 | 0 | 0 | 2.875 | ZFYVE19 | AW015263 |
| 25815 | 0 | 0 | 0 | 1.625 | BOC | W72626 |
| 25816 | 0 | 0 | 0 | 8.25 | TMEM41A | BE644935 |
| 25854 | 0 | 0 | 0 | 5.875 | VANGL2 | AB033041 |
| 25872 | 0 | 0 | 0 | 6.25 | MRVI1 | N66571 |
| 25879 | 0 | 0 | 0 | 22.75 | BRD4 | AA702437 |
| 25890 | 87 | 0 | 0 | 1.75 | PRICKLE1 | N98595 |
| 26055 | 0 | 0 | 0 | 3.125 | SMEK2 | AA541716 |
| 26321 | 0 | 25 | 74 | 12.875 | ZCCHC7 | BG291039 |
| 26389 | 28 | 0 | 0 | 9.125 | ZFAT | BF941325 |
| 26474 | 0 | 0 | 0 | 2.875 | ZFAND2A | AI984061 |
| 26559 | 0 | 0 | 0 | 1 | TAPT1 | AI239899 |
| 26729 | 0 | 0 | 0 | 22.5 | FAM101B | BG036514 |
| 26750 | 0 | 0 | 0 | 5.5 | DMKN | AA706316 |
| 26955 | 0 | 0 | 0 | 5 | MAP3K3 | BG231756 |
| 27236 | 0 | 0 | 0 | 6.625 | PPP1R3E | AK024489 |
| 27331 | 0 | 0 | 0 | 5.75 | — | AI302271 |
| 27487 | 0 | 0 | 0 | 10.875 | hCG_2008140 | AW149809 |
| 27873 | 0 | 43 | 0 | 7.25 | UTP15 | AA046406 |
| 27888 | 0 | 0 | 0 | 8.75 | BCL9L | AL353962 |
| 27915 | 0 | 0 | 0 | 7.875 | CREM | AL552470 |
| 27997 | 0 | 0 | 0 | 10.125 | C9orf126 | AI832363 |
| 28042 | 0 | 0 | 0 | 10 | UPB1 | AI770035 |
| 28091 | 0 | 95 | 0 | 0.75 | FMO2 | AI758223 |
| 28163 | 0 | 0 | 0 | 2.75 | TLE3 | BE967118 |
| 28281 | 0 | 0 | 0 | 0.625 | C6orf226 | AI636501 |
| 28316 | 0 | 67 | 62 | 9.125 | NKAP | T87628 |
| 28462 | 0 | 0 | 0 | 6.5 | — | BG054835 |
| 28542 | 0 | 0 | 0 | 1.625 | ZSWIM7 | BE645222 |
| 28601 | 0 | 0 | 0 | 1.875 | — | BE673677 |
| 28700 | 0 | 34 | 0 | 8.375 | RGL3 | AI379517 |
| 28739 | 0 | 0 | 65 | 4.5 | CWF19L2 | BE857467 |
| 28795 | 39 | 0 | 0 | 7.75 | — | AI028602 |
| 28954 | 0 | 0 | 64 | 4.625 | — | AI702450 |
| 29105 | 0 | 16 | 0 | 10.625 | GATA6 | AI762621 |
| 29117 | 0 | 0 | 71 | 3.75 | JPH3 | AL537395 |
| 29214 | 24 | 0 | 0 | 9.625 | FAM26F | AV734646 |
| 29502 | 21 | 0 | 0 | 10 | C12orf76 | AI870880 |
| 29858 | 0 | 0 | 0 | 7.875 | BOC | BF447871 |
| 29949 | 0 | 0 | 0 | 7.75 | KDM4B | AI265747 |
| 29992 | 0 | 0 | 0 | 11.625 | THAP6 | AI199523 |
| 30150 | 69 | 0 | 0 | 4 | LOC730098 | AI203673 |
| 30320 | 70 | 0 | 0 | 8.5 | BRUNOL5 | BE503640 |
| 30345 | 94 | 0 | 0 | 0.875 | C9orf100 | BG028209 |
| 30397 | 0 | 0 | 0 | 10.125 | LOC100130938 | AW139393 |
| 30428 | 73 | 0 | 0 | 3.5 | — | BF433830 |
| 30513 | 0 | 0 | 0 | 14.75 | TUBB1 | N63244 |
| 30517 | 0 | 46 | 0 | 6.875 | — | AI340341 |
| 30543 | 0 | 66 | 0 | 4.375 | RNF182 | AI884906 |
| 30559 | 0 | 0 | 0 | 5.125 | LOC387647 | AW118878 |
| 30682 | 0 | 0 | 0 | 2.125 | — | BF111276 |
| 30758 | 61 | 0 | 0 | 5 | — | AI861874 |
| 30874 | 0 | 0 | 0 | 1.75 | — | W69743 |
| 30903 | 0 | 0 | 38 | 7.875 | CDAN1 | AI951606 |
| 31011 | 0 | 0 | 0 | 13.625 | ZSCAN2 | AW206602 |
| 31220 | 0 | 0 | 0 | 17.75 | PAP2D | AF131783 |
| 31296 | 35 | 0 | 36 | 44 | — | AI554926 |
| 31325 | 0 | 12 | 48 | 17.75 | — | BF591615 |
| 31501 | 0 | 33 | 91 | 9.75 | ADH4 | AV651117 |
| 31544 | 0 | 35 | 2 | 20.625 | JAM3 | AF356518 |
| 31826 | 0 | 0 | 0 | 0.75 | PNMAL2 | AW299761 |
| 31897 | 0 | 0 | 0 | 1.125 | PRSS27 | AW170323 |
| 31902 | 0 | 0 | 0 | 8.625 | PVRL2 | BE867789 |
| 31946 | 6 | 0 | 0 | 21.625 | LOC283174 | BF527412 |
| 32012 | 0 | 0 | 11 | 11.25 | — | AK026459 |
| 32031 | 0 | 0 | 59 | 5.25 | ISLR2 | AW007241 |
| 32067 | 0 | 0 | 0 | 3.25 | KIAA1161 | AB032987 |
| 32082 | 46 | 0 | 0 | 6.875 | LOC100009676 | BC003066 |
| 32152 | 0 | 0 | 0 | 0.75 | RANBP10 | AV717059 |
| 32361 | 0 | 0 | 0 | 8.5 | — | AK027226 |
| 32470 | 0 | 93 | 0 | 1 | PROCA1 | AL137531 |
| 32506 | 0 | 0 | 67 | 4.25 | PARP6 | AL122091 |
| 32536 | 0 | 0 | 0 | 13.5 | — | AL365407 |
| 32751 | 13 | 0 | 0 | 11 | — | AU154942 |
| 32959 | 0 | 0 | 0 | 5.5 | — | AF143887 |
| 32976 | 0 | 0 | 0 | 6.375 | — | AK022197 |
| 32998 | 0 | 0 | 0 | 0.625 | — | AK024243 |
| 33198 | 0 | 0 | 0 | 11.375 | — | AK025118 |
| 33318 | 0 | 0 | 0 | 6.125 | ERBB4 | AK024204 |
| 33519 | 0 | 0 | 0 | 19.375 | — | AK025173 |
| 33553 | 0 | 0 | 66 | 4.375 | — | AL137552 |
| 33944 | 0 | 0 | 0 | 5.25 | — | AL137318 |
| 34212 | 0 | 0 | 0 | 8 | ZNF124 | AB046850 |
| 34213 | 0 | 81 | 45 | 9.5 | — | AF065869 |
| 34280 | 0 | 0 | 0 | 24.25 | — | S51397 |
| 34315 | 0 | 98 | 33 | 8.875 | — | AK022113 |
| 34333 | 0 | 0 | 0 | 20.5 | PCGEM1 | AF223389 |
| 34396 | 0 | 0 | 0 | 8.375 | — | AL157496 |
| 34491 | 18 | 0 | 22 | 28 | HHLA2 | AK027132 |
| 34497 | 0 | 0 | 87 | 1.75 | KRTAP9-3 | AJ406947 |
| 34503 | 0 | 0 | 0 | 5.875 | KRTAP4-9 | AJ406941 |
| 34517 | 0 | 0 | 0 | 11.875 | RNASE7 | AJ131212 |
| 34555 | 0 | 0 | 0 | 12.125 | NT5DC3 | AK002128 |
| 34695 | 0 | 0 | 0 | 18.75 | — | L21961 |
| 34845 | 0 | 51 | 0 | 6.25 | — | R52023 |
| 34884 | 93 | 0 | 0 | 1 | MAP4 | AI078534 |
| 34890 | 0 | 0 | 0 | 8.5 | — | BF594695 |
| 34896 | 8 | 0 | 0 | 11.625 | — | AI393725 |
| 35065 | 0 | 0 | 58 | 5.375 | — | AI224578 |
| 35189 | 0 | 0 | 0 | 5.25 | GLT8D4 | AI452595 |
| 35268 | 0 | 0 | 0 | 6.75 | FBXL4 | BF571480 |
| 35410 | 0 | 0 | 0 | 3.625 | — | AW960145 |
| 35564 | 0 | 0 | 0 | 2 | PLA2R1 | BE048919 |
| 35645 | 0 | 20 | 0 | 10.125 | MAP3K7IP1 | AW592369 |
| 36052 | 0 | 0 | 0 | 9.125 | PDE1A | AW614381 |
| 36312 | 0 | 0 | 0 | 4.125 | — | AW003845 |
| 36506 | 0 | 0 | 0 | 19.75 | FRMPD3 | AL133943 |
| 36539 | 44 | 0 | 0 | 7.125 | ALKBH1 | AI922200 |
| 36574 | 0 | 94 | 0 | 0.875 | LOC389857 | BE466872 |
| 36694 | 76 | 0 | 0 | 12.75 | H1FNT | AW013835 |
| 36701 | 0 | 0 | 0 | 25.625 | — | AI769104 |
| 36721 | 54 | 0 | 0 | 5.875 | — | BF511686 |
| 36758 | 0 | 0 | 0 | 4.75 | — | W60647 |
| 36801 | 0 | 0 | 0 | 6.375 | TMC5 | AI738488 |
| 37073 | 0 | 84 | 0 | 2.125 | — | BF222867 |
| 37229 | 0 | 61 | 84 | 7.125 | ADAMTS6 | N71063 |
| 37239 | 0 | 0 | 0 | 3.75 | — | BF509605 |
| 37291 | 0 | 0 | 0 | 7 | LOC100130494 | AW027469 |
| | | | | | /// | |
| | | | | | LOC728448 | |
| 37559 | 0 | 0 | 77 | 3 | SLC25A36 | AW514168 |
| 37573 | 0 | 0 | 81 | 2.5 | WDR16 | AW673231 |
| 37590 | 0 | 0 | 0 | 4.875 | LOC100129286 | AI732275 |
| 37630 | 0 | 0 | 0 | 7.625 | — | AI684424 |
| 37647 | 0 | 0 | 0 | 9.875 | — | AI732280 |
| 37649 | 0 | 48 | 94 | 7.5 | MMAA | R15084 |
| 37735 | 0 | 0 | 0 | 3.375 | NBPF8 | N62721 |
| 37752 | 40 | 0 | 0 | 7.625 | — | AI873296 |
| 37789 | 0 | 0 | 0 | 1.375 | — | AI341258 |
| 37872 | 0 | 0 | 0 | 9.5 | ADPRHL1 | AI243209 |
| 37962 | 0 | 0 | 0 | 5.375 | — | BF514993 |
| 37966 | 0 | 0 | 0 | 11.25 | ZNF818P | AI651641 |
| 38158 | 0 | 0 | 0 | 8.875 | WDR42A | AL134577 |
| 38289 | 0 | 0 | 0 | 0.125 | — | AI684833 |
| 38445 | 0 | 0 | 0 | 13.75 | TRAPPC2L | AW827150 |
| 38536 | 0 | 0 | 0 | 11.25 | — | BF382322 |
| 38756 | 0 | 0 | 73 | 3.5 | — | AI674059 |
| 39035 | 0 | 62 | 0 | 4.875 | ABCC3 | AI375341 |
| 39136 | 0 | 0 | 0 | 3.125 | FAM118B | AI632973 |
| 39161 | 0 | 0 | 0 | 10.375 | LOC728705 | AW451176 |
| 39237 | 99 | 26 | 1 | 22.125 | PTPRA | AA652313 |
| 39241 | 0 | 0 | 0 | 7 | — | AW043836 |
| 39320 | 0 | 0 | 0 | 11 | — | AA478981 |
| 39340 | 0 | 0 | 0 | 11 | IL12RB1 | AI637915 |
| 39361 | 0 | 41 | 39 | 20.125 | — | AW275049 |
| 39458 | 0 | 0 | 0 | 1.75 | LOC401320 | AI221073 |
| 39477 | 0 | 0 | 0 | 1.875 | — | BF591259 |
| 39504 | 0 | 0 | 0 | 20.375 | — | AI694557 |
| 39625 | 0 | 0 | 0 | 8.125 | LOC728842 | AI693407 |
| 39720 | 0 | 0 | 0 | 5.375 | — | AI766224 |
| 39744 | 53 | 0 | 0 | 6 | — | AI675753 |
| 39747 | 12 | 0 | 0 | 11.125 | PM20D1 | AA918425 |
| 39832 | 0 | 0 | 0 | 6.5 | POLR2J4 | AI821720 |
| 40016 | 14 | 0 | 44 | 18.875 | — | AA348683 |
| 40021 | 0 | 0 | 0 | 9.125 | — | AI921894 |
| 40081 | 0 | 0 | 0 | 9 | — | N74924 |
| 40134 | 84 | 0 | 0 | 2.125 | C9orf57 | AW274388 |
| 40198 | 0 | 0 | 0 | 4 | — | N63808 |
| 40217 | 0 | 0 | 0 | 12.625 | — | AA668261 |
| 40245 | 79 | 0 | 0 | 3.875 | — | AW445087 |
| 40305 | 0 | 0 | 0 | 8.875 | — | AI184609 |
| 40422 | 92 | 0 | 0 | 1.125 | ERI2 | AI688141 |
| 40613 | 0 | 0 | 0 | 13.125 | — | AA001970 |
| 40905 | 0 | 0 | 28 | 9.125 | — | AV654572 |
| 40958 | 0 | 27 | 23 | 19 | LMO7 | AA702962 |
| 41050 | 38 | 0 | 0 | 7.875 | — | AW236797 |
| 41149 | 0 | 0 | 80 | 2.625 | SKAP2 | BE671499 |
| 41215 | 0 | 96 | 0 | 0.625 | — | AW276866 |
| 41302 | 0 | 97 | 0 | 0.5 | — | AI668696 |
| 41374 | 0 | 0 | 0 | 2.125 | — | N27112 |
| 41443 | 0 | 8 | 0 | 11.625 | — | BE221330 |
| 41489 | 58 | 0 | 0 | 5.375 | FLJ22536 | H14782 |
| 41500 | 0 | 0 | 0 | 1.5 | KLHL23 | BE873351 |
| 41719 | 0 | 0 | 0 | 1.75 | — | AA770235 |
| 41901 | 95 | 0 | 0 | 0.75 | ZNF81 | AI028309 |
| 41911 | 0 | 59 | 63 | 10 | SYTL5 | AW263497 |
| 42228 | 0 | 0 | 0 | 10.875 | CACNA1E | R15004 |
| 42244 | 0 | 0 | 0 | 18.375 | NRG4 | BF793585 |
| 42287 | 0 | 0 | 0 | 1.25 | LOC120376 | AI590055 |
| 42333 | 16 | 0 | 0 | 18.75 | C11orf17 | AI933861 |
| 42415 | 0 | 0 | 0 | 15.875 | — | H11894 |
| 42424 | 0 | 0 | 0 | 3.5 | — | AL043482 |
| 42683 | 0 | 54 | 0 | 5.875 | — | AI332638 |
| 42726 | 0 | 0 | 0 | 3.625 | — | R46483 |
| 42965 | 0 | 0 | 0 | 10.75 | — | AW118707 |
| 43009 | 0 | 17 | 14 | 21.375 | — | BE044588 |
| 43041 | 0 | 0 | 6 | 11.875 | — | AA453526 |
| 43100 | 0 | 0 | 0 | 3.25 | CCDC93 | AA504256 |
| 43210 | 0 | 80 | 0 | 2.625 | USP49 | BF727345 |
| 43219 | 0 | 18 | 0 | 10.375 | — | AA806070 |
| 43415 | 0 | 70 | 0 | 3.875 | FANCB | BE550133 |
| 43420 | 0 | 0 | 40 | 7.625 | MGC40069 | AI684979 |
| 43565 | 0 | 0 | 0 | 1.5 | ZNF599 | AI222019 |
| 43618 | 0 | 0 | 0 | 4.375 | NR1H4 | AI051958 |
| 43777 | 0 | 0 | 46 | 6.875 | — | N35099 |
| 43889 | 0 | 29 | 0 | 9 | FBLL1 | AA868464 |
| 44334 | 0 | 79 | 0 | 2.75 | — | AW291120 |
| 44407 | 0 | 0 | 0 | 17.125 | — | AI026951 |
| 44411 | 75 | 0 | 0 | 3.25 | C17orf28 | AL554277 |
| 44451 | 0 | 0 | 0 | 2 | — | AA404996 |
| 44582 | 0 | 0 | 0 | 1 | — | BG250907 |
| 44584 | 0 | 0 | 0 | 4.625 | LOC440354 | BG180003 |
| | | | | | /// | |
| | | | | | LOC595101 | |
| | | | | | /// | |
| | | | | | LOC641298 | |
| | | | | | /// | |
| | | | | | LOC728423 | |
| | | | | | /// | |
| | | | | | LOC729513 | |
| | | | | | /// SMG1 | |
| 44717 | 0 | 0 | 0 | 2.125 | ADAM32 | NM_145004 |
| 44778 | 63 | 0 | 0 | 4.75 | SLC25A43 | AK094254 |
| 44808 | 0 | 58 | 15 | 16.125 | CLEC12A /// | NM_138337 |
| | | | | | CLEC12B | |
| 45271 | 0 | 64 | 41 | 12.125 | RECQL4 | NM_004260 |
| 45312 | 0 | 0 | 24 | 9.625 | GPR78 | NM_080819 |
| 45349 | 0 | 0 | 0 | 15 | PTK6 | NM_005975 |
| 45403 | 0 | 0 | 0 | 0.375 | RASEF | NM_152573 |
| 45407 | 0 | 0 | 0 | 4.25 | ZNF441 | NM_152355 |
| 45430 | 29 | 0 | 0 | 9 | OXER1 | AB083055 |
| 45440 | 50 | 0 | 0 | 6.375 | PCDHAC1 | NM_031882 |
| 45451 | 0 | 0 | 0 | 7 | BRWD3 | NM_153252 |
| 45814 | 82 | 0 | 0 | 2.375 | RHEBL1 | NM_144593 |
| 45877 | 0 | 0 | 0 | 6.75 | C14orf126 | NM_080664 |
| 45973 | 0 | 0 | 0 | 5.75 | C7orf33 | NM_145304 |
| 45996 | 0 | 0 | 0 | 1.625 | SNX21 | CA447177 |
| 46126 | 0 | 0 | 0 | 9.25 | C3orf15 | AB063297 |
| 46539 | 0 | 0 | 61 | 5 | KCNMB1 | BC025707 |
| 46892 | 0 | 0 | 0 | 4 | ST3GAL3 | AF425864 |
| 47027 | 0 | 0 | 0 | 7.875 | SCML4 | BC033286 |
| 47038 | 96 | 0 | 0 | 0.625 | ZNF479 | AF277624 |
| 47085 | 0 | 0 | 0 | 6.375 | IL31RA | AF106913 |
| 47106 | 0 | 0 | 0 | 10.75 | PPP1R1C | AF494535 |
| 47115 | 55 | 0 | 0 | 18.125 | SORBS2 | AF396457 |
| 47298 | 0 | 0 | 0 | 8.25 | ATN1 | Z22814 |
| 47323 | 0 | 0 | 0 | 11.625 | C14orf34 | BC008034 |
| 47374 | 0 | 0 | 0 | 3 | — | BI524781 |
| 47470 | 0 | 0 | 0 | 1 | C22orf42 | CA775385 |
| 47482 | 0 | 63 | 89 | 6.25 | CSNK1A1 | BQ025347 |
| 47691 | 9 | 0 | 0 | 13.375 | CCBL2 /// | AA460960 |
| | | | | | LOC100131735 | |
| | | | | | /// RBMX | |
| 47784 | 4 | 0 | 0 | 19.75 | SCML4 | CA448477 |
| 47858 | 0 | 100 | 0 | 0.125 | LOC284513 | AW169311 |
| 47868 | 0 | 32 | 21 | 18.625 | LOC100129637 | AA758799 |
| 47897 | 0 | 0 | 0 | 16.75 | — | AI860021 |
| 47925 | 88 | 0 | 0 | 1.625 | FLJ42709 | AK095719 |
| 47926 | 33 | 0 | 0 | 14.375 | FLJ42709 | AK095719 |
| 47941 | 0 | 0 | 0 | 3.875 | — | AK092802 |
| 48143 | 0 | 0 | 0 | 6.75 | — | BC040670 |
| 48159 | 0 | 0 | 0 | 5.25 | — | AK097488 |
| 48237 | 59 | 0 | 0 | 7.375 | HCG11 | AK024111 |
| 48265 | 0 | 0 | 0 | 1.25 | FANCB | BC043596 |
| 48343 | 0 | 0 | 0 | 9.625 | — | W95489 |
| 48404 | 0 | 0 | 0 | 5.125 | — | AW273830 |
| 48540 | 0 | 0 | 0 | 6.25 | POM121L8P | BC035394 |
| 48544 | 0 | 0 | 0 | 0.875 | NFIA | AI220445 |
| 48783 | 0 | 24 | 10 | 21 | — | BG109597 |
| 48789 | 15 | 0 | 0 | 10.75 | CP | AL556703 |
| 48977 | 0 | 90 | 0 | 1.375 | IGHG1 | S55277 |
| 49079 | 66 | 0 | 0 | 15.5 | — | BC033250 |
| 49168 | 0 | 0 | 0 | 10.5 | PIK3R6 | AK091819 |
| 49203 | 22 | 0 | 0 | 9.875 | — | AF147412 |
| 49237 | 0 | 0 | 0 | 18 | SREBF1 | S66168 |
| 49250 | 0 | 0 | 0 | 9.5 | PLK5P | AK054808 |
| 49357 | 27 | 0 | 0 | 9.25 | — | AI678088 |
| 49377 | 45 | 0 | 0 | 7 | LOC644135 | BC038719 |
| 49385 | 0 | 0 | 0 | 6.125 | — | BU176936 |
| 49404 | 0 | 0 | 68 | 4.125 | LOC285954 | AK096266 |
| 49429 | 0 | 0 | 0 | 1.75 | NFYC | AL713771 |
| 49714 | 74 | 0 | 0 | 3.375 | — | BC039376 |
| 49727 | 0 | 0 | 0 | 8.75 | RGNEF | AB082529 |
| 49761 | 0 | 0 | 0 | 0.125 | — | AI885742 |
| 49797 | 0 | 0 | 0 | 10.75 | NSUN4 | BC016907 |
| 49967 | 0 | 55 | 0 | 5.75 | — | BC041865 |
| 50106 | 0 | 0 | 0 | 5.625 | — | AL832499 |
| 50574 | 0 | 0 | 47 | 6.75 | — | AF075113 |
| 50602 | 0 | 0 | 76 | 3.125 | VWA3B | BM981856 |
| 50640 | 0 | 0 | 0 | 5.625 | LOC283682 | BC043440 |
| 50678 | 36 | 0 | 52 | 24.5 | hCG_2015435 | BC035235 |
| 50696 | 0 | 0 | 0 | 0.25 | — | BC042427 |
| 50722 | 0 | 68 | 31 | 12.875 | LOC692247 | BG772667 |
| 50754 | 80 | 0 | 0 | 2.625 | ARAP2 | BC031283 |
| 50833 | 0 | 0 | 0 | 3.375 | — | BC036630 |
| 50955 | 0 | 0 | 0 | 9.75 | — | BC040600 |
| 50987 | 0 | 0 | 96 | 1.125 | — | AL512742 |
| 51032 | 0 | 0 | 0 | 7 | — | AL359058 |
| 51135 | 0 | 0 | 0 | 6.375 | — | AK055464 |
| 51177 | 0 | 0 | 0 | 6.25 | — | BC041050 |
| 51229 | 0 | 0 | 0 | 17 | — | AF147390 |
| 51342 | 0 | 19 | 19 | 20.5 | — | BC020562 |
| 51588 | 0 | 57 | 7 | 17.25 | — | BC043373 |
| 51611 | 71 | 0 | 0 | 3.75 | — | AK021715 |
| 51618 | 0 | 0 | 0 | 6.125 | — | BQ002451 |
| 51639 | 0 | 0 | 0 | 4.125 | — | BC043439 |
| 51802 | 0 | 83 | 0 | 2.25 | — | BC032028 |
| 51928 | 0 | 0 | 95 | 0.75 | — | AF087974 |
| 51940 | 0 | 0 | 0 | 5.375 | — | BC038205 |
| 51960 | 0 | 0 | 0 | 4.375 | DEFB107A /// | AF540979 |
| | | | | | DEFB107B | |
| 51981 | 0 | 0 | 0 | 3.75 | CTA-221G9.4 | AL832019 |
| 52033 | 0 | 52 | 0 | 6.125 | LOC285456 | AK094992 |
| 52052 | 0 | 0 | 0 | 1.625 | MTBP | AL832671 |
| 52063 | 0 | 0 | 0 | 11.75 | TNNT2 | AL832707 |
| 52114 | 0 | 0 | 0 | 7.375 | LOC283140 | AK095275 |
| 52135 | 0 | 0 | 0 | 14.125 | LOC283045 | AK095019 |
| 52354 | 0 | 0 | 0 | 12.875 | LOC146795 | AK057377 |
| 52414 | 89 | 0 | 0 | 1.5 | CCDC36 | AK058049 |
| 52547 | 0 | 0 | 0 | 9.5 | OFCC1 | AF520802 |
| 52828 | 0 | 0 | 16 | 10.625 | LOC91431 | AF075091 |
| 52968 | 0 | 0 | 0 | 3.625 | — | AF085916 |
| 52985 | 0 | 2 | 17 | 22.875 | — | AL833305 |
| 53014 | 0 | 0 | 0 | 4.75 | — | AL831875 |
| 53063 | 0 | 0 | 0 | 0.25 | — | AL137307 |
| 53125 | 0 | 0 | 0 | 0.75 | — | AL117464 |
| 53155 | 0 | 0 | 97 | 0.5 | DNAH1 | AK093347 |
| 53225 | 3 | 0 | 0 | 24 | — | AL831906 |
| 53309 | 0 | 0 | 0 | 9 | CLN6 | D17218 |
| 53332 | 0 | 0 | 92 | 1.125 | OR2L2 | X64978 |
| 53339 | 0 | 0 | 0 | 12.375 | OR9A1P | X64982 |
| 53614 | 0 | 0 | 0 | 9.875 | C6orf41 | AI028608 |
| 53702 | 67 | 0 | 0 | 4.25 | — | BC025967 |
| 53707 | 0 | 0 | 25 | 9.5 | — | BC020935 |
| 53860 | 0 | 30 | 0 | 8.875 | LOC284440 | BC037856 |
| 54119 | 0 | 88 | 60 | 17.25 | — | BC035112 |
| 54150 | 0 | 0 | 0 | 3.25 | SLC25A18 | BC016954 |
| 54206 | 0 | 0 | 0 | 6.75 | NCRNA00119 | BC036463 |
| 54295 | 0 | 87 | 0 | 1.75 | WIPI2 | BC016912 |
| 54482 | 0 | 0 | 0 | 4.875 | — | BC030089 |
| 54485 | 5 | 0 | 0 | 12 | C20orf62 | BC030259 |
| 54541 | 0 | 0 | 0 | 0.125 | TMPRSS2 | BC015819 |

Table 5b

| Gene Symbol | Gene Title |
|---|---|
| SPAG7 | sperm associated antigen 7 |
| MSN | moesin |
| SPARC | secreted protein, acidic, cysteine-rich (osteonectin) |
| ARPC1A | actin related protein 2/3 complex, subunit 1A, 41 kDa |
| MDH1 | malate dehydrogenase 1, NAD (soluble) |
| SMARCC1 | SWI/SNF related, matrix associated, actin dependent regulator of chromatin, subfamily c, |
| | member 1 |
| ITGB5 | integrin, beta 5 |
| IGFBP7 | insulin-like growth factor binding protein 7 |
| NDUFB8/SEC31B | NADH dehydrogenase (ubiquinone) 1 beta subcomplex, 8, 19 kDa /// SEC31 homolog B |
| | (S. cerevisiae) |
| ANXA4 | annexin A4 |
| CUL3 | cullin 3 |
| DHX15 | DEAH (Asp-Glu-Ala-His) box polypeptide 15 |
| SOX4 | SRY (sex determining region Y)-box 4 |
| MAPKAPK2 | mitogen-activated protein kinase-activated protein kinase 2 |
| SCRN1 | secernin 1 |
| NQO1 | NAD(P)H dehydrogenase, quinone 1 |
| AAMP | angio-associated, migratory cell protein |
| DUSP3 | dual specificity phosphatase 3 |
| DLG5 | discs, large homolog 5 (Drosophila) |
| CEACAM5 | carcinoembryonic antigen-related cell adhesion molecule 5 |
| CTDSPL | CTD (carboxy-terminal domain, RNA polymerase II, polypeptide A) small phosphatase-like |
| FARP1 | FERM, RhoGEF (ARHGEF) and pleckstrin domain protein 1 (chondrocyte-derived) |
| C7orf28A | chromosome 7 open reading frame 28A |
| EGFR | epidermal growth factor receptor (erythroblastic leukemia viral (v-erb-b) oncogene homolog, |
| | avian) |
| SFRP1 | secreted frizzled-related protein 1 |
| COPS8 | COP9 constitutive photomorphogenic homolog subunit 8 (Arabidopsis) |
| ACBD3 | acyl-Coenzyme A binding domain containing 3 |
| UNC119B | unc-119 homolog B (C. elegans) |
| CRMP1 | collapsin response mediator protein 1 |
| HMGN4 | high mobility group nucleosomal binding domain 4 |
| SNRPD1 | small nuclear ribonucleoprotein D1 polypeptide 16 kDa |
| INPP5K | inositol polyphosphate-5-phosphatase K |
| DNAJB12 | DnaJ (Hsp40) homolog, subfamily B, member 12 |
| KLHL21 | kelch-like 21 (Drosophila) |
| FXR2 | fragile X mental retardation, autosomal homolog 2 |
| TLE1 | transducin-like enhancer of split 1 (E(sp1) homolog, Drosophila) |
| UNC119 | unc-119 homolog (C. elegans) |
| EDEM1 | ER degradation enhancer, mannosidase alpha-like 1 |
| LMAN1 | lectin, mannose-binding, 1 |
| SCNN1A | sodium channel, nonvoltage-gated 1 alpha |
| ARL4D | ADP-ribosylation factor-like 4D |
| DAPK3 | death-associated protein kinase 3 |
| HEPH | hephaestin |
| RAP1GAP | RAP1 GTPase activating protein |
| CDC6 | cell division cycle 6 homolog (S. cerevisiae) |
| MAOB | monoamine oxidase B |
| PREP | prolyl endopeptidase |
| ACPP | acid phosphatase, prostate |
| EML2 | echinoderm microtubule associated protein like 2 |
| FGF2 | fibroblast growth factor 2 (basic) |
| MRC1 /// MRC1L1 | mannose receptor, C type 1 /// mannose receptor, C type 1-like 1 |
| GAS1 | growth arrest-specific 1 |
| ITGB3 | integrin, beta 3 (platelet glycoprotein IIIa, antigen CD61) |
| TTF1 | transcription termination factor, RNA polymerase I |
| RRAD | Ras-related associated with diabetes |
| ICAM3 | intercellular adhesion molecule 3 |
| SNAP91 | synaptosomal-associated protein, 91 kDa homolog (mouse) |
| CENPA | centromere protein A |
| GPC4 | glypican 4 |
| PGC | progastricsin (pepsinogen C) |
| PIGA | phosphatidylinositol glycan anchor biosynthesis, class A |
| BAIAP2 | BAI1-associated protein 2 |
| OVGP1 | oviductal glycoprotein 1, 120 kDa |
| KIAA0586 | KIAA0586 |
| SPIB | Spi-B transcription factor (Spi-1/PU.1 related) |
| BDKRB2 | bradykinin receptor B2 |
| IL15 | interleukin 15 |
| CHML | choroideremia-like (Rab escort protein 2) |
| RHAG | Rh-associated glycoprotein |
| SERPINA6 | serpin peptidase inhibitor, clade A (alpha-1 antiproteinase, antitrypsin), member 6 |
| MLANA | melan-A |
| ITGA2B | integrin, alpha 2b (platelet glycoprotein IIb of IIb/IIIa complex, antigen CD41) |
| PML | promyelocytic leukemia |
| AKR1B10 | aldo-keto reductase family 1, member B10 (aldose reductase) |
| KCNQ3 | potassium voltage-gated channel, KQT-like subfamily, member 3 |
| MAGEC1 | melanoma antigen family C, 1 |
| SIX3 | SIX homeobox 3 |
| CHRNB2 | cholinergic receptor, nicotinic, beta 2 (neuronal) |
| CXorf1 | chromosome X open reading frame 1 |
| GRIA3 | glutamate receptor, ionotrophic, AMPA 3 |
| DLEC1 | deleted in lung and esophageal cancer 1 |
| G6PC | glucose-6-phosphatase, catalytic subunit |
| IL8RA | interleukin 8 receptor, alpha |
| SAA4 | serum amyloid A4, constitutive |
| GRM5 | glutamate receptor, metabotropic 5 |
| ALOX15 | arachidonate 15-lipoxygenase |
| USP34 | ubiquitin specific peptidase 34 |
| SLC22A2 | solute carrier family 22 (organic cation transporter), member 2 |
| SHOX | short stature homeobox |
| XRCC2 | X-ray repair complementing defective repair in Chinese hamster cells 2 |
| GAGE3 | G antigen 3 |
| CACNB4 | calcium channel, voltage-dependent, beta 4 subunit |
| — | — |
| IFNW1 | interferon, omega 1 |
| SCN7A | sodium channel, voltage-gated, type VII, alpha |
| ZNF157 | zinc finger protein 157 |
| AVPR2 | arginine vasopressin receptor 2 |
| LIPE | lipase, hormone-sensitive |
| C4BPB | complement component 4 binding protein, beta |
| ADAM22 | ADAM metallopeptidase domain 22 |
| TH | tyrosine hydroxylase |
| PCK1 | phosphoenolpyruvate carboxykinase 1 (soluble) |
| FRMD4A | FERM domain containing 4A |
| CSNK1D | casein kinase 1, delta |
| NCOR2 | nuclear receptor co-repressor 2 |
| NR2F2 | nuclear receptor subfamily 2, group F, member 2 |
| CDC42EP3 | CDC42 effector protein (Rho GTPase binding) 3 |
| BAD | BCL2-associated agonist of cell death |
| HIP1R | huntingtin interacting protein 1 related |
| TGFB1I1 | transforming growth factor beta 1 induced transcript 1 |
| ABCG2 | ATP-binding cassette, sub-family G (WHITE), member 2 |
| PADI2 | peptidyl arginine deiminase, type II |
| CHST3 | carbohydrate (chondroitin 6) sulfotransferase 3 |
| TRIM9 | tripartite motif-containing 9 |
| PCGF1 | polycomb group ring finger 1 |
| PRKCQ | protein kinase C, theta |
| IGFBP3 | insulin-like growth factor binding protein 3 |
| EFNA3 | ephrin-A3 |
| PSG1 | pregnancy specific beta-1-glycoprotein 1 |
| — | — |
| ABCC8 | ATP-binding cassette, sub-family C (CFTR/MRP), member 8 |
| SFRS17A | splicing factor, arginine/serine-rich 17A |
| SGCD | sarcoglycan, delta (35 kDa dystrophin-associated glycoprotein) |
| SLC24A1 | solute carrier family 24 (sodium/potassium/calcium exchanger), member 1 |
| FETUB | fetuin B |
| DTNA | dystrobrevin, alpha |
| GLRA3 | glycine receptor, alpha 3 |
| MCM7 | minichromosome maintenance complex component 7 |
| MGLL | monoglyceride lipase |
| PTPRU | protein tyrosine phosphatase, receptor type, U |
| IFNA2 | interferon, alpha 2 |
| MTAP | methylthioadenosine phosphorylase |
| GABARAPL1 /// | GABA(A) receptor-associated protein like 1 /// GABA(A) receptors associated protein like 3 |
| GABARAPL3 | (pseudogene) |
| NFIB | nuclear factor I/B |
| CXCR6 | chemokine (C—X—C motif) receptor 6 |
| RAPSN | receptor-associated protein of the synapse |
| IGH3 /// IGHA1 /// | immunoglobulin heavy locus /// immunoglobulin heavy constant alpha 1 /// immunoglobulin |
| IGHA2 /// IGHD /// | heavy constant alpha 2 (A2m marker) /// immunoglobulin heavy constant delta /// |
| IGHG1 /// IGHG3 /// | immunoglobulin heavy constant gamma 1 (G1m marker) /// immunoglobulin heavy constant ga |
| IGHG4 /// IGHM /// | |
| IGHV3-23 /// IGHV4- | |
| 31 /// | |
| LOC100126583 /// | |
| LOC642131 /// | |
| LOC652128 /// | |
| VSIG6 | |
| IGHA1 /// IGHG1 /// | immunoglobulin heavy constant alpha 1 /// immunoglobulin heavy constant gamma 1 (G1m |
| LOC100133862 | marker) /// similar to hCG1773549 |
| KCND3 | potassium voltage-gated channel, Shal-related subfamily, member 3 |
| ZNF471 | zinc finger protein 471 |
| PEG10 | paternally expressed 10 |
| CAV1 | caveolin 1, caveolae protein, 22 kDa |
| TUBA4A | tubulin, alpha 4a |
| SETD3 | SET domain containing 3 |
| ARAP1 | ArfGAP with RhoGAP domain, ankyrin repeat and PH domain 1 |
| — | — |
| ZEB1 | zinc finger E-box binding homeobox 1 |
| SORT1 | sortilin 1 |
| ZNF282 | zinc finger protein 282 |
| FBXO9 | F-box protein 9 |
| APOOL | Apolipoprotein O-like |
| ATG2A | ATG2 autophagy related 2 homolog A (S. cerevisiae) |
| ZC3H7B | zinc finger CCCH-type containing 7B |
| TMCC1 | transmembrane and coiled-coil domain family 1 |
| MXRA8 | matrix-remodelling associated 8 |
| DGCR6L | DiGeorge syndrome critical region gene 6-like |
| CCDC22 | coiled-coil domain containing 22 |
| MAN1C1 | mannosidase, alpha, class 1C, member 1 |
| NOV | nephroblastoma overexpressed gene |
| TRPM1 | Transient receptor potential cation channel, subfamily M, member 1 |
| HLX | H2.0-like homeobox |
| AZU1 | azurocidin 1 |
| USP19 | ubiquitin specific peptidase 19 |
| AZI1 | 5-azacytidine induced 1 |
| AHCTF1 | AT hook containing transcription factor 1 |
| CLCN4 | chloride channel 4 |
| IGKV4-1 | immunoglobulin kappa variable 4-1 |
| — | — |
| MCF2 | MCF.2 cell line derived transforming sequence |
| MUC3B | mucin 3B, cell surface associated |
| TMC6 | transmembrane channel-like 6 |
| — | — |
| DST | dystonin |
| B3GNTL1 | UDP-GlcNAc:betaGal beta-1,3-N-acetylglucosaminyltransferase-like 1 |
| EIF3M | eukaryotic translation initiation factor 3, subunit M |
| — | — |
| ZNF391 | zinc finger protein 391 |
| LOC100128640 | Hypothetical protein LOC100128640 |
| — | — |
| GRIK2 | Glutamate receptor, ionotropic, kainate 2 |
| LOC100134355 /// | similar to Primase, DNA, polypeptide 2 (58 kDa) /// primase, DNA, polypeptide 2 (58 kDa) |
| PRIM2 | |
| DTX2 /// | deltex homolog 2 (Drosophila) /// hypothetical protein LOC100134197 |
| LOC100134197 | |
| USF2 | upstream transcription factor 2, c-fos interacting |
| TLL2 | tolloid-like 2 |
| MRPS11 | mitochondrial ribosomal protein S11 |
| — | — |
| CPN2 | carboxypeptidase N, polypeptide 2 |
| HCG2P7 | HLA complex group 2 pseudogene 7 |
| IGL@ | immunoglobulin lambda locus |
| C19orf10 | chromosome 19 open reading frame 10 |
| — | — |
| TAL1 | T-cell acute lymphocytic leukemia 1 |
| FASN | fatty acid synthase |
| GBX1 | gastrulation brain homeobox 1 |
| NTN3 | Netrin 3 |
| — | — |
| — | — |
| ESR1 | estrogen receptor 1 |
| ZFX | zinc finger protein, X-linked |
| CYB561 | cytochrome b-561 |
| LOC642131 | Similar to hCG1812074 |
| CEACAM5 | carcinoembryonic antigen-related cell adhesion molecule 5 |
| SHMT1 | serine hydroxymethyltransferase 1 (soluble) |
| — | — |
| FOLH1 | folate hydrolase (prostate-specific membrane antigen) 1 |
| FAM55C | family with sequence similarity 55, member C |
| — | — |
| AKAP6 | A kinase (PRKA) anchor protein 6 |
| — | — |
| CPSF7 | cleavage and polyadenylation specific factor 7, 59 kDa |
| DUS1L | dihydrouridine synthase 1-like (S. cerevisiae) |
| TSEN34 | tRNA splicing endonuclease 34 homolog (S. cerevisiae) |
| INF2 | inverted formin, FH2 and WH2 domain containing |
| C14orf159 | chromosome 14 open reading frame 159 |
| TRAPPC2L | trafficking protein particle complex 2-like |
| NUDT9 | nudix (nucleoside diphosphate linked moiety X)-type motif 9 |
| TRIAP1 | TP53 regulated inhibitor of apoptosis 1 |
| CERK | ceramide kinase |
| COMMD10 | COMM domain containing 10 |
| LYRM4 | LYR motif containing 4 |
| MAGEH1 | melanoma antigen family H, 1 |
| LRRC40 | leucine rich repeat containing 40 |
| PUS1 | pseudouridylate synthase 1 |
| SMUG1 | single-strand-selective monofunctional uracil-DNA glycosylase 1 |
| TSPAN15 | tetraspanin 15 |
| TMEM51 | transmembrane protein 51 |
| WDR3 | WD repeat domain 3 |
| C1orf66 | chromosome 1 open reading frame 66 |
| PYCRL | pyrroline-5-carboxylate reductase-like |
| KRT23 | keratin 23 (histone deacetylase inducible) |
| PID1 | phosphotyrosine interaction domain containing 1 |
| TRPV2 | transient receptor potential cation channel, subfamily V, member 2 |
| CEP76 | centrosomal protein 76 kDa |
| SNIP1 | Smad nuclear interacting protein 1 |
| NXN | nucleoredoxin |
| RTN3 | reticulon 3 |
| CYP20A1 | cytochrome P450, family 20, subfamily A, polypeptide 1 |
| ZNF767 | zinc finger family member 767 |
| LRP1B | low density lipoprotein-related protein 1B (deleted in tumors) |
| HAUS2 | HAUS augmin-like complex, subunit 2 |
| ANTXR1 | anthrax toxin receptor 1 |
| SPATA6 | spermatogenesis associated 6 |
| FLJ42627 | hypothetical LOC645644 |
| SPTLC3 | serine palmitoyltransferase, long chain base subunit 3 |
| GUCY1B2 | guanylate cyclase 1, soluble, beta 2 |
| CCDC40 | coiled-coil domain containing 40 |
| IFT122 | intraflagellar transport 122 homolog (Chlamydomonas) |
| PRG3 | proteoglycan 3 |
| FLJ11292 | hypothetical protein FLJ11292 |
| — | — |
| METTL5 | methyltransferase like 5 |
| — | — |
| ANGPTL4 | angiopoietin-like 4 |
| SLC25A32 | solute carrier family 25, member 32 |
| — | — |
| CLDN18 | claudin 18 |
| CCDC70 | coiled-coil domain containing 70 |
| HRH4 | histamine receptor H4 |
| FGF14 | fibroblast growth factor 14 |
| P2RX2 | purinergic receptor P2X, ligand-gated ion channel, 2 |
| PCDHB12 | protocadherin beta 12 |
| CDCA3 | cell division cycle associated 3 |
| GDF15 | growth differentiation factor 15 |
| RAB35 | RAB35, member RAS oncogene family |
| — | — |
| DENND2A | DENN/MADD domain containing 2A |
| FAM131A | family with sequence similarity 131, member A |
| SCIN | scinderin |
| — | — |
| KCTD2 | potassium channel tetramerisation domain containing 2 |
| FXR2 | fragile X mental retardation, autosomal homolog 2 |
| ARHGAP25 | Rho GTPase activating protein 25 |
| STK10 | serine/threonine kinase 10 |
| KIAA1644 | KIAA1644 |
| THRAP3 | thyroid hormone receptor associated protein 3 |
| COX4NB | COX4 neighbor |
| BAALC | brain and acute leukemia, cytoplasmic |
| C20orf7 | chromosome 20 open reading frame 7 |
| CLDN12 | claudin 12 |
| COX15 | COX15 homolog, cytochrome c oxidase assembly protein (yeast) |
| NAT14 | N-acetyltransferase 14 (GCN5-related, putative) |
| COMMD2 | COMM domain containing 2 |
| CLPX | ClpX caseinolytic peptidase X homolog (E. coli) |
| TMEM108 | transmembrane protein 108 |
| NLRP12 | NLR family, pyrin domain containing 12 |
| CHRDL2 | chordin-like 2 |
| CCL28 | chemokine (C-C motif) ligand 28 |
| IL20 | interleukin 20 |
| DPYSL5 | dihydropyrimidinase-like 5 |
| BOC | Boc homolog (mouse) |
| — | — |
| FKSG49 | FKSG49 |
| LOC100131508 | PRO2122 |
| AGPAT9 | 1-acylglycerol-3-phosphate O-acyltransferase 9 |
| NT5C1A | 5′-nucleotidase, cytosolic IA |
| PCDHAC2 | protocadherin alpha subfamily C, 2 |
| BIRC6 | baculoviral IAP repeat-containing 6 |
| PIGY | phosphatidylinositol glycan anchor biosynthesis, class Y |
| FAM100B | family with sequence similarity 100, member B |
| TNKS1BP1 | tankyrase 1 binding protein 1, 182 kDa |
| PREX1 | phosphatidylinositol-3,4,5-trisphosphate-dependent Rac exchange factor 1 |
| EXOC4 | exocyst complex component 4 |
| RAB3D | RAB3D, member RAS oncogene family |
| CHD2 | chromodomain helicase DNA binding protein 2 |
| RBM18 | RNA binding motif protein 18 |
| SLC39A10 | solute carrier family 39 (zinc transporter), member 10 |
| IGF1R | insulin-like growth factor 1 receptor |
| GLE1 | GLE1 RNA export mediator homolog (yeast) |
| ARID2 | AT rich interactive domain 2 (ARID, RFX-like) |
| C13orf37 | chromosome 13 open reading frame 37 |
| LOC401504 | Hypothetical gene supported by AK091718 |
| ZFYVE19 | zinc finger, FYVE domain containing 19 |
| BOC | Boc homolog (mouse) |
| TMEM41A | transmembrane protein 41A |
| VANGL2 | vang-like 2 (van gogh, Drosophila) |
| MRVI1 | murine retrovirus integration site 1 homolog |
| BRD4 | bromodomain containing 4 |
| PRICKLE1 | prickle homolog 1 (Drosophila) |
| SMEK2 | SMEK homolog 2, suppressor of mek1 (Dictyostelium) |
| ZCCHC7 | zinc finger, CCHC domain containing 7 |
| ZFAT | zinc finger and AT hook domain containing |
| ZFAND2A | zinc finger, AN1-type domain 2A |
| TAPT1 | transmembrane anterior posterior transformation 1 |
| FAM101B | family with sequence similarity 101, member B |
| DMKN | dermokine |
| MAP3K3 | mitogen-activated protein kinase kinase kinase 3 |
| PPP1R3E | protein phosphatase 1, regulatory (inhibitor) subunit 3E |
| — | — |
| hCG_2008140 | hypothetical LOC729614 |
| UTP15 | UTP15, U3 small nucleolar ribonucleoprotein, homolog (S. cerevisiae) |
| BCL9L | B-cell CLL/lymphoma 9-like |
| CREM | cAMP responsive element modulator |
| C9orf126 | chromosome 9 open reading frame 126 |
| UPB1 | ureidopropionase, beta |
| FMO2 | flavin containing monooxygenase 2 (non-functional) |
| TLE3 | transducin-like enhancer of split 3 (E(sp1) homolog, Drosophila) |
| C6orf226 | chromosome 6 open reading frame 226 |
| NKAP | NFKB activating protein |
| — | — |
| ZSWIM7 | zinc finger, SWIM-type containing 7 |
| — | — |
| RGL3 | ral guanine nucleotide dissociation stimulator-like 3 |
| CWF19L2 | CWF19-like 2, cell cycle control (S. pombe) |
| — | — |
| — | — |
| GATA6 | GATA binding protein 6 |
| JPH3 | junctophilin 3 |
| FAM26F | family with sequence similarity 26, member F |
| C12orf76 | chromosome 12 open reading frame 76 |
| BOC | Boc homolog (mouse) |
| KDM4B | Lysine (K)-specific demethylase 4B |
| THAP6 | THAP domain containing 6 |
| LOC730098 | similar to chemokine (C-C motif) ligand 27 |
| BRUNOL5 | bruno-like 5, RNA binding protein (Drosophila) |
| C9orf100 | chromosome 9 open reading frame 100 |
| LOC100130938 | hypothetical protein LOC100130938 |
| — | — |
| TUBB1 | tubulin, beta 1 |
| — | — |
| RNF182 | ring finger protein 182 |
| LOC387647 | patched domain containing 3 pseudogene |
| — | — |
| — | — |
| — | — |
| CDAN1 | Congenital dyserythropoietic anemia, type I |
| ZSCAN2 | zinc finger and SCAN domain containing 2 |
| PAP2D | phosphatidic acid phosphatase type 2 |
| — | — |
| — | — |
| ADH4 | alcohol dehydrogenase 4 (class II), pi polypeptide |
| JAM3 | junctional adhesion molecule 3 |
| PNMAL2 | PNMA-like 2 |
| PRSS27 | protease, serine 27 |
| PVRL2 | poliovirus receptor-related 2 (herpesvirus entry mediator B) |
| LOC283174 | hypothetical LOC283174 |
| — | — |
| ISLR2 | immunoglobulin superfamily containing leucine-rich repeat 2 |
| KIAA1161 | KIAA1161 |
| LOC100009676 | hypothetical LOC100009676 |
| RANBP10 | RAN binding protein 10 |
| — | — |
| PROCA1 | protein interacting with cyclin A1 |
| PARP6 | poly (ADP-ribose) polymerase family, member 6 |
| — | — |
| — | — |
| — | — |
| — | — |
| — | — |
| — | — |
| ERBB4 | v-erb-a erythroblastic leukemia viral oncogene homolog 4 (avian) |
| — | — |
| — | — |
| — | — |
| ZNF124 | zinc finger protein 124 |
| — | — |
| — | — |
| — | — |
| PCGEM1 | prostate-specific transcript 1 (non-protein coding) |
| — | — |
| HHLA2 | HERV-H LTR-associating 2 |
| KRTAP9-3 | keratin associated protein 9-3 |
| KRTAP4-9 | keratin associated protein 4-9 |
| RNASE7 | ribonuclease, RNase A family, 7 |
| NT5DC3 | 5′-nucleotidase domain containing 3 |
| — | — |
| — | — |
| MAP4 | microtubule-associated protein 4 |
| — | — |
| — | — |
| — | — |
| GLT8D4 | glycosyltransferase 8 domain containing 4 |
| FBXL4 | F-box and leucine-rich repeat protein 4 |
| — | — |
| PLA2R1 | phospholipase A2 receptor 1, 180 kDa |
| MAP3K7IP1 | mitogen-activated protein kinase kinase kinase 7 interacting protein 1 |
| PDE1A | phosphodiesterase 1A, calmodulin-dependent |
| — | — |
| FRMPD3 | FERM and PDZ domain containing 3 |
| ALKBH1 | alkB, alkylation repair homolog 1 (E. coli) |
| LOC389857 | hypothetical protein |
| H1FNT | H1 histone family, member N, testis-specific |
| — | — |
| — | — |
| — | — |
| TMC5 | transmembrane channel-like 5 |
| — | — |
| ADAMTS6 | ADAM metallopeptidase with thrombospondin type 1 motif, 6 |
| — | — |
| LOC100130494 /// | hypothetical protein LOC100130494 /// peptidylprolyl isomerase E pseudogene |
| LOC728448 | |
| SLC25A36 | Solute carrier family 25, member 36 |
| WDR16 | WD repeat domain 16 |
| LOC100129286 | Hypothetical protein LOC100129286 |
| — | — |
| — | — |
| MMAA | methylmalonic aciduria (cobalamin deficiency) cbIA type |
| NBPF8 | neuroblastoma breakpoint family, member 8 |
| — | — |
| — | — |
| ADPRHL1 | ADP-ribosylhydrolase like 1 |
| — | — |
| ZNF818P | zinc finger protein 818 pseudogene |
| WDR42A | WD repeat domain 42A |
| — | — |
| TRAPPC2L | trafficking protein particle complex 2-like |
| — | — |
| — | — |
| ABCC3 | ATP-binding cassette, sub-family C (CFTR/MRP), member 3 |
| FAM118B | family with sequence similarity 118, member B |
| LOC728705 | hypothetical protein LOC728705 |
| PTPRA | Protein tyrosine phosphatase, receptor type, A |
| — | — |
| — | — |
| IL12RB1 | interleukin 12 receptor, beta 1 |
| — | — |
| LOC401320 | Hypothetical LOC401320 |
| — | — |
| — | — |
| LOC728842 | hypothetical LOC728842 |
| — | — |
| — | — |
| PM20D1 | peptidase M20 domain containing 1 |
| POLR2J4 | polymerase (RNA) II (DNA directed) polypeptide J4, pseudogene |
| — | — |
| — | — |
| — | — |
| C9orf57 | chromosome 9 open reading frame 57 |
| — | — |
| — | — |
| — | — |
| — | — |
| ERI2 | exoribonuclease 2 |
| — | — |
| — | — |
| LMO7 | LIM domain 7 |
| — | — |
| SKAP2 | Src kinase associated phosphoprotein 2 |
| — | — |
| — | — |
| — | — |
| — | — |
| FLJ22536 | hypothetical locus LOC401237 |
| KLHL23 | kelch-like 23 (Drosophila) |
| — | — |
| ZNF81 | zinc finger protein 81 |
| SYTL5 | synaptotagmin-like 5 |
| CACNA1E | calcium channel, voltage-dependent, R type, alpha 1E subunit |
| NRG4 | neuregulin 4 |
| LOC120376 | Uncharacterized protein LOC120376 |
| C11orf17 | chromosome 11 open reading frame 17 |
| — | — |
| — | — |
| — | — |
| — | — |
| — | — |
| — | — |
| — | — |
| CCDC93 | coiled-coil domain containing 93 |
| USP49 | ubiquitin specific peptidase 49 |
| — | — |
| FANCB | Fanconi anemia, complementation group B |
| MGC40069 | Hypothetical protein MGC40069 |
| ZNF599 | zinc finger protein 599 |
| NR1H4 | nuclear receptor subfamily 1, group H, member 4 |
| — | — |
| FBLL1 | fibrillarin-like 1 |
| — | — |
| — | — |
| C17orf28 | chromosome 17 open reading frame 28 |
| — | — |
| — | — |
| LOC440354 /// | PI-3-kinase-related kinase SMG-1 pseudogene /// PI-3-kinase-related kinase SMG-1 |
| LOC595101 /// | pseudogene /// SMG1 homolog, phosphatidylinositol 3-kinase-related kinase pseudogene /// |
| LOC641298 /// | hypothetical LOC728423 /// similar to PI-3-kinase-related kinase SMG-1 /// SMG1 homol |
| LOC728423 /// | |
| LOC729513 /// | |
| SMG1 | |
| ADAM32 | ADAM metallopeptidase domain 32 |
| SLC25A43 | solute carrier family 25, member 43 |
| CLEC12A /// | C-type lectin domain family 12, member A /// C-type lectin domain family 12, member B |
| CLEC12B | |
| RECQL4 | RecQ protein-like 4 |
| GPR78 | G protein-coupled receptor 78 |
| PTK6 | PTK6 protein tyrosine kinase 6 |
| RASEF | RAS and EF-hand domain containing |
| ZNF441 | zinc finger protein 441 |
| OXER1 | oxoeicosanoid (OXE) receptor 1 |
| PCDHAC1 | protocadherin alpha subfamily C, 1 |
| BRWD3 | bromodomain and WD repeat domain containing 3 |
| RHEBL1 | Ras homolog enriched in brain like 1 |
| C14orf126 | chromosome 14 open reading frame 126 |
| C7orf33 | chromosome 7 open reading frame 33 |
| SNX21 | sorting nexin family member 21 |
| C3orf15 | chromosome 3 open reading frame 15 |
| KCNMB1 | potassium large conductance calcium-activated channel, subfamily M, beta member 1 |
| ST3GAL3 | ST3 beta-galactoside alpha-2,3-sialyltransferase 3 |
| SCML4 | sex comb on midleg-like 4 (Drosophila) |
| ZNF479 | zinc finger protein 479 |
| IL31RA | interleukin 31 receptor A |
| PPP1R1C | protein phosphatase 1, regulatory (inhibitor) subunit 1C |
| SORBS2 | sorbin and SH3 domain containing 2 |
| ATN1 | atrophin 1 |
| C14orf34 | chromosome 14 open reading frame 34 |
| — | — |
| C22orf42 | chromosome 22 open reading frame 42 |
| CSNK1A1 | Casein kinase 1, alpha 1 |
| CCBL2 /// | cysteine conjugate-beta lyase 2 /// similar to RNA binding motif protein, X-linked /// |
| | RNA binding |
| LOC100131735 /// | motif protein, X-linked |
| RBMX | |
| SCML4 | sex comb on midleg-like 4 (Drosophila) |
| LOC284513 | hypothetical protein LOC284513 |
| LOC100129637 | hypothetical LOC100129637 |
| — | — |
| FLJ42709 | hypothetical LOC441094 |
| FLJ42709 | hypothetical LOC441094 |
| — | — |
| — | — |
| — | — |
| HCG11 | HLA complex group 11 |
| FANCB | Fanconi anemia, complementation group B |
| — | — |
| — | — |
| POM121L8P | POM121 membrane glycoprotein-like 8 (rat) pseudogene |
| NFIA | Nuclear factor I/A |
| — | — |
| CP | ceruloplasmin (ferroxidase) |
| IGHG1 | Immunoglobulin heavy constant gamma 1 (G1m marker) |
| — | — |
| PIK3R6 | phosphoinositide-3-kinase, regulatory subunit 6 |
| — | — |
| SREBF1 | sterol regulatory element binding transcription factor 1 |
| PLK5P | polo-like kinase 5 pseudogene |
| — | — |
| LOC644135 | hypothetical LOC644135 |
| — | — |
| LOC285954 | hypothetical LOC285954 |
| NFYC | nuclear transcription factor Y, gamma |
| — | — |
| RGNEF | Rho-guanine nucleotide exchange factor |
| — | — |
| NSUN4 | NOL1/NOP2/Sun domain family, member 4 |
| — | — |
| — | — |
| — | — |
| VWA3B | von Willebrand factor A domain containing 3B |
| LOC283682 | Hypothetical protein LOC283682 |
| hCG_2015435 | hypothetical protein LOC100128554 |
| — | — |
| LOC692247 | hypothetical locus LOC692247 |
| ARAP2 | ArfGAP with RhoGAP domain, ankyrin repeat and PH domain 2 |
| — | — |
| — | — |
| — | — |
| — | — |
| — | — |
| — | — |
| — | — |
| — | — |
| — | — |
| — | — |
| — | — |
| — | — |
| — | — |
| — | — |
| — | — |
| DEFB107A /// | defensin, beta 107A /// defensin, beta 107B |
| DEFB107B | |
| CTA-221G9.4 | KIAA1671 protein |
| LOC285456 | hypothetical LOC285456 |
| MTBP | Mdm2, transformed 3T3 cell double minute 2, p53 binding protein (mouse) binding protein, |
| | 104 kDa |
| TNNT2 | troponin T type 2 (cardiac) |
| LOC283140 | hypothetical protein LOC283140 |
| LOC283045 | hypothetical protein LOC283045 |
| LOC146795 | hypothetical protein LOC146795 |
| CCDC36 | coiled-coil domain containing 36 |
| OFCC1 | orofacial cleft 1 candidate 1 |
| LOC91431 | prematurely terminated mRNA decay factor-like |
| — | — |
| — | — |
| — | — |
| — | — |
| — | — |
| DNAH1 | dynein, axonemal, heavy chain 1 |
| — | — |
| CLN6 | ceroid-lipofuscinosis, neuronal 6, late infantile, variant |
| OR2L2 | olfactory receptor, family 2, subfamily L, member 2 |
| OR9A1P | olfactory receptor, family 9, subfamily A, member 1 pseudogene |
| C6orf41 | chromosome 6 open reading frame 41 |
| — | — |
| — | — |
| LOC284440 | hypothetical LOC284440 |
| — | — |
| SLC25A18 | solute carrier family 25 (mitochondrial carrier), member 18 |
| NCRNA00119 | non-protein coding RNA 119 |
| WIPI2 | WD repeat domain, phosphoinositide interacting 2 |
| — | — |
| C20orf62 | chromosome 20 open reading frame 62 |
| TMPRSS2 | transmembrane protease, serine 2 |

What is claimed is:

1. A method of identifying a rheumatoid arthritis patient that is a candidate for treatment with an human interleukin-6 receptor antibody or a rheumatoid arthritis patient that should be excluded from treatment, the method comprising:
providing an RNA nucleic acid sample obtained from peripheral blood lymphocytes from the patient;
determining the level of expression of at least one gene product encoded by a gene set forth in Table 1, Table 2, or Table 3 that is associated with a therapeutic response to treatment with IL-6 receptor antibody; wherein when the level exceeds the threshold value, the level of the biomarker is indicative of a patient that is a candidate for treatment with the human interleukin-6 receptor antibody; or that a patient that should be excluded from treatment.

2. The method of claim 1, wherein the method comprises detecting the level of expression of gene products encoded by at least two, three, four, five, six, seven, eight, nine, ten, twenty, thirty, or forty or more, of the genes set forth in Table 1, Table 2, or Table 3.

3. The method of claim 1, wherein the step of determining the level of expression comprises an amplification reaction.

4. The method of claim 3, wherein the amplification reaction is a quantitative RT-PCR.

5. The method of claim 1, further comprising recording the correlation of the presence of the SNP with a positive response to treatment with IL-6 receptor antibody.

6. The method of claim 5, further comprising administering IL-6 receptor antibody to the patient.

7. A method of identifying a rheumatoid arthritis patient that is a candidate for treatment with an human interleukin-6 receptor antibody or patient that should be excluded from treatment, the method comprising:
providing a serum sample from the patient or a sample comprising protein from peripheral blood lymphocytes;
determining the level of expression of at least one gene product encoded by a gene set forth in Table 1, Table 2, or Table 3 that is associated with a therapeutic response to treatment with IL-6 receptor antibody.

8. A diagnostic device comprising two or more nucleic acid probes attached to a solid surface to detect RNA expression levels of two or more biomarkers set forth in Table 1, Table 2, or Table 3.

9. The diagnostic device of claim 8, wherein device comprises probes to detect RNA expression level of three, four, five, six, seven, eight, nine, ten, twenty, thirty, or forty or more, of the biomarkers set forth in Table 1, Table 2, or Table 3.

10. A method of identifying a rheumatoid arthritis patient that is a candidate for treatment with an human interleukin-6 receptor antibody or a rheumatoid arthritis patient that should be excluded from treatment, the method comprising:
providing an RNA nucleic acid sample obtained from peripheral blood lymphocytes from the patient;
determining the level of expression of at least two gene products having a value>0 in column C of Table 5, or the level the level of expression of at least two gene products having a value>0 in column D of Table 5, or the level of expression of at least two gene products having a value>0 in column E of Table 5, or the level of expression of at least two gene products having a value>0 in column F of Table 5, or the level of expression of at least two gene products having a value>0 in column G of Table 5, or the level of expression of at least two gene products having a value>0 in column H of Table 5, or the level of expression of at least two gene products having a value>0 in column I of Table 5, or the level of expression of at least two gene products having a value>0 in column J of Table 5;
wherein the linear combination of the expression levels of the at least two gene products. that exceeds a threshold value is indicative of a patient that is a candidate for treatment with the human interleukin-6 receptor antibody; or that a patient that should be excluded from treatment.
